(19) United States
(12) Patent Application Publication
(10) Pub. No.: US 2006/0281113 A1
(43) Pub. Date: Dec. 14, 2006

(54) Accessible polynucleotide libraries and methods of use thereof

(76) Inventors: George Church, Brookline, MA (US); Edmund R. Pitcher, Lexington, MA (US)

Correspondence Address: FISH & NEAVE IP GROUP;ROPES & GRAY LLP, ONE INTERNATIONAL PLACE, BOSTON, MA, 02110-2624, US

(21) Appl. No.: 11/436,478

(22) Filed: May 17, 2006

Related U.S. Application Data
(60) Provisional application No. 60/682,148, filed on May 18, 2005.

(51) Int. Cl. C12Q 1/68; C12P 19/34; C12M 1/34

(52) U.S. Cl. 435/6; 435/91.2; 435/287.2

(57) ABSTRACT

Disclosed are methods of assembling nucleic acid constructs from component parts in a manner that is not dependent on the sequence of the component parts. The methods may be used to assemble large nucleic acid constructs from multiple component parts in one or more reactions. The methods may also be used to assemble two or more nucleic acid constructs in the same reaction mixture. In exemplary embodiments, the methods involve formation of a Holliday junction or bridge structure using a junction oligonucleotide.

### RELATED APPLICATIONS

This application claims the benefit of U.S. Provisional Application No. 60/682,148, filed May 18, 2005, which application is hereby incorporated by reference in its entirety.

### BACKGROUND

A key aim of biotechnology is a rational approach to the construction of new biomaterials that can be used for analytical, industrial or therapeutic purposes. Using the techniques of recombinant DNA chemistry, it is now common for DNA sequences to be replicated and amplified from nature and for those sequences to then be disassembled into component parts which are then recombined or reassembled into new DNA sequences. However, reliance on naturally available sequences significantly limits the possibilities that may be explored by researchers. While it is now possible for short DNA sequences to be directly synthesized from individual nucleosides, it has been generally impractical to directly construct large segments or assemblies of DNA sequences larger than about 400 base pairs. As a consequence, larger segments of DNA are generally constructed from component parts and segments which can be purchased, cloned or synthesized individually and then assembled into the DNA molecule desired.

Current methods for constructing larger nucleic acids from component parts require sequence based methods for assembly, such as, restriction endonuclease cleavage followed by ligation. These sequence based methods limit the number of different combinations that can be produced from a given set of components because each new combination requires careful planning and design to obtain a desired product linking the individual components into a new arrangement.

Recently, as disclosed in the Registry of Biological Parts (world wide web at parts.mit.edu), methods have been developed which permit ligation of DNAs of diverse function using standardized technology. Physical parts in the DNA repository, called “Biobricks,” are designed to be assembled into systems and composite parts using normal cloning techniques based on restriction enzymes, purification, ligation, and transformation. It has been proposed to improve the system to permit seamless construction of combinations of parts, and such a system is described in BioBricks++: Simplifying Assembly of Standard DNA Components, Austin Che, 2004 (world wide web at austin.che.name/docs/bbpp.pdf)

BioBricks++ uses commercially available restriction enzymes and standard biological techniques for assembling modules. Standard DNA modules are packaged with a standard prefix and suffix DNA sequence containing several restriction enzyme sites, which are used for different module operations. The modules are adapted to permit arbitrary assembly of any two modules by blunt end ligation, and the assemblies can be made seamless, or “scarless,” with no extra intervening bases inserted between the modules and no bases missing.

Techniques for the manufacture of diverse and complex combinations of DNAs that are sequence independent, connect parts together seamlessly, can be executed using standard protocols, involve minimal independent operations per connection, and that assemble multiple parts in a specific desired order in a single solution would be a very effective way to explore DNA, RNA and protein structure. Such a technique would enable production of a family of designs providing tens, hundreds, or multiple thousands of different constructs embodying, for example, multiple, evolutionarily independent design approaches adapted for selection, screening, or further diversification. This would permit the discovery of DNA, RNA, protein, new metabolic pathways, and cellular constructs that may have therapeutic or commercial value in a much more efficient and systematic manner.

The widespread use of gene and genome synthesis technology is hampered by limitations such as high cost, lack of automation, and the necessity to rely on sequence based methods for cloning and amplification. It is therefore an object of this invention to provide practical, economical methods of synthesizing custom polynucleotides and large genetic systems. It is a further object to provide efficient methods for assembling polynucleotides from standardized parts that are not dependent on the sequence of the individual parts to be joined together, can be joined seamlessly, and can be multiplexed.

### SUMMARY

The invention provides compositions and methods for the preparation of polynucleotide constructs having a predetermined sequence or set of sequences. More particularly, the invention provides methods and compositions exploiting construction polynucleotides, typically synthetic polynucleotides, specially designed as “building blocks” to facilitate assembly into high fidelity larger polynucleotides of predefined sequence such as genes, simple and complex regulatory elements, transcription units, multi-component constructs encoding enzymes and regulatory machinery for implementing a series of chemical changes in metabolic pathways, vectors, plasmids, artificial chromosomes, and portions or all of complete genomes.

The inventions disclosed herein provide methods enabling the bioengineer to design virtually and then to embody as functional DNA essentially any desired DNA sequence. The inventions are characterized by techniques which permit ligation of any one building block to any other, (including one or more copies of itself), in any order, seamlessly (without leaving a scar of unwanted intervening bases or deleting wanted bases), and, in preferred embodiments making multiple connections simultaneously. Alternatively, the inventions provide “tool kits” or apparatus for the manufacture of simple and complex DNA encoded biological systems. These kits comprising tens to thousands to millions of individual different construction polynucleotide building blocks in one or more reservoirs, and additional reservoirs of primer pairs and junction oligonucleotides. These, preferably together with hardware such as dispensing machinery, automated equipment for conducting lhgations, PCR amplifications, DNA digestions, etc., and software for implementing or at least tracking the assembly procedures, permit the bioengineer to plan and then to build any one of an enormous number of bioconstructs. These synthetic constructs may be used in various ways, e.g., integrated into a cellular chassis for expression, or expressed in an in vitro display system to obtain a protein having a desired combination of properties.

In one embodiment, each “construction polynucleotide” comprises a medial segment which embodies the information content of the DNA, and is a candidate for inclusion in a larger construct. The medial segment or region may have essentially any sequence, and its length may vary widely. It may be flanked by additional polynucleotide flanking sequences, which are, in various embodiments, used to purify, amplify, retrieve from a mixture, and, together with junction oligonucleotides, to direct which end of which polynucleotide building block will be joined to which end of another. These flanking sequences typically also are designed so as to be selectively and readily removed as disclosed herein, e.g., by building into their structure an endonuclease recognition site or other structure that will permit restriction at the proper location immediately adjacent the medial segment, and using other strategies as disclosed herein. Preferably, multiple different strategies for removal are embodied in the construction polynucleotide collection so as to permit multiplexed operation—assembly of multiple polynucleotides in a predetermined order in a single reaction mixture. Preferably, at least orthogonal chemistries are used to remove the 3′ flanking sequences and the 5′ flanking sequences so as to permit in a given operation the removal of either one but not the other.

Thus, in one aspect, the invention provides compositions of construction polynucleotides that may be joined together in any order to form one or more longer polynucleotide constructs. The construction polynucleotides comprise a medial segment and 3′ and 5′ flanking sequences that may be selectably removed. In an exemplary embodiment, at least a portion of the 3′ and/or 5′ flanking sequences of a construction polynucleotide may be used to form a single stranded overhang. The 5′ and 3′ flanking regions may comprise one or more sets of primer binding sites. In an exemplary embodiment, the invention provides a composition comprising a mixture of construction polynucleotides having nested primer binding sites for at least two sets of primers. Different construction polynucleotides in the mixture may be isolated by selective amplification from the mixture using a unique combination of the primer pairs. Alternatively, the construction polynucleotides may comprise affinity sequences in the 3′ and/or 5′ flanking regions. Affinity sequences may be aptamer sequences or hybridization sequences that can be used to selectively isolate a construction polynucleotide from a mixture.

In another aspect, the invention provides compositions of junction oligonucleotides that may be used to facilitate a junction reaction between two or more construction polynucleotide. The junction oligonucleotides may comprise sequences that are complementary to 3′ and 5′ terminal portions of the medial segments of two or more construction polynucleotides (or the same polynucleotide for making tandem repeats). Alternatively, the junction oligonucleotides may comprise sequences that are complementary to at least a portion of the 3′ and 5′ flanking sequences of two or more construction polynucleotides (or the same polynucleotide for making tandem repeats). The junction oligonucleotides may optionally comprise 3′ and 5′ flanking sequences having, for example, primer binding sites that may be used for amplification of the junction oligonucleotides. The 3′ and 5′ flanking sequences may be selectively removable by chemical or enzymatic means.

In one embodiment, the invention provides a set of construction polynucleotides, junction oligonucleotides and optionally, primer pairs that may be used for constructing one or a plurality of polynucleotide constructs. The primer pairs may be used to amplify and/or isolate a construction polynucleotide and/or a junction oligonucleotide from a mixture of oligonucleotides. The set of oligonucleotides may be provided, for example, in a multi-well plate that stores the oligonucleotides in an addressable manner.

In another aspect, the invention provides junction assembly methods for connecting together two or more construction polynucleotides. The junction assembly methods utilize a junction oligonucleotide to adjacently align two construction polynucleotides so that they may be covalently connected under ligation conditions. In various embodiments, the junction oligonucleotides may be complementary to regions of the medial sequence of two or more construction polynucleotides (or a single construction polynucleotide to form tandem repeats) or complementary to at least a portion of the 3′ and 5′ flanking regions of two or more construction polynucleotides (or a single construction polynucleotide to form tandem repeats). In one embodiment, the junction oligonucleotide hybridizes to at least portions of the flanking regions of two construction polynucleotides and forms a Holliday junction. The Holliday junction may be cleaved with resolvase and the medial segments of the two construction polynucleotides connected together under ligation conditions. In another embodiment, the junction oligonucleotide hybridizes to at least portions of one strand of the 5′ and 3′ flanking sequences of two construction polynucleotides and additionally hybridizes to ˜4-8 base pairs of the medial sequences of the opposite strands of the construction polynucleotides thereby forming a bridge structure. The portion of the junction oligonucleotide that binds to the medial sequences may comprise universal or degenerate bases such that the hybridization reaction is minimally dependent on the sequence of the medial sequences. After formation of the bridge, the medial segments of the construction polynucleotides may be joined together under ligation conditions. The junction assembly methods may be used to prepare large polynucleotide constructs, e.g., comprising at least 2, 4, 6, 8, 10, 20, 30, 40, 50, 75, 100, or more, construction polynucleotides linked together. Additionally, the junction assembly methods may be used to prepare a large variety of different polynucleotide constructs, e.g., comprising at least 2, 4, 6, 8, 10, 20, 30, 40, 50, 75, 100, or more, constructs. When preparing multiple and/or large polynucleotide constructs, the junction assembly reactions may be conducted in a single reaction mixture or may be conducted in multiple reactions in parallel or serial optionally using a hierarchical assembly strategy.

In yet another embodiment, the invention provides methods of preparing one or a group of polynucleotide constructs simultaneously by assembly of double stranded construction polynucleotides comprising at least a sense strand consisting of a sequence to be incorporated within the polynucleotide construct(s). The method involves chemically synthesizing in parallel on a surface, severing from the surface, and purifying a plurality of different junction oligonucleotides defining high fidelity sequences complementary to respective 3′ and 5′ termini of construction polynucleotides to be joined. These junction oligonucleotides are designed on an ad hoc basis and synthesized rapidly and easily once the identity and sequence of joinder of construction polynucleotides are known. The construction polynucleotides may be retrieved from a library comprising one or more mixtures of candidate building blocks, e.g., blunt ended double stranded building blocks or members having flanking sequences. Optionally, the construction polynucleotides are provided by selective amplification from a mixture of candidate construction polynucleotides using selectively removable primer sites. The construction polynucleotides and junction oligonucleotides then are mixed together under hybridizing conditions to produce one or a plurality of intermediates comprising serially arranged construction polynucleotides linked by bridging junction oligonucleotides. These then are subjected to a polymerase or a ligase to prepare the polynucleotide construct(s).

This method may be used to produce inexpensively and rapidly polynucleotide construct comprises more than 1 Kb, 5 Kb, 10 Kb or 100 Kb, or to prepare a plurality of different polynucleotide constructs simultaneously by providing first and second pluralities of double stranded construction polynucleotides and chemically synthesizing first and second different junction oligonucleotides.

The different polynucleotide constructs may comprise different candidates for expression and testing for a preselected property, such as DNAs encoding a plurality of different open reading frames or sequences encoding a plurality of different enzymes and including different regulatory elements to be tested for activity as a functional synthetic metabolic pathway.

In one aspect, the invention provides a method of directed scarless ligation of a construction polynucleotide to another to produce a larger polynucleotide construct, the method comprising: (a) providing double stranded construction polynucleotides comprising a medial segment comprising a DNA sequence for inclusion within a said larger polynucleotide construct and left and right flanking terminal double stranded sequences of a length sufficient to permit selective hybridization of a complementary DNA thereto; (b) creating a 3′ single stranded overhang corresponding to a 3′ flanking region in a first said construction polynucleotide and a 5′ single stranded overhang corresponding to a 5′ flanking region in a second said construction polynucleotide while retaining the medial segments of said polynucleotides intact and free of residual flanking sequence bases; (c) contacting the construction polynucleotides under hybridization conditions with a junction oligonucleotide comprising sequence complementary to at least a portion of both said construction polynucleotides to form a complex wherein the two medial segments are aligned end to end; and (d) exposing the complex to ligation conditions, thereby forming a larger polynucleotide construct comprising fused said medial segments.

In certain embodiments of the above described method, step (c) may be conducted by contacting said first and second construction polynucleotides with a junction oligonucleotide complementary to at least a portion of the medial segments of both said construction polynucleotides. In other embodiments of the above described method, step (c) may be conducted by contacting said first and second construction polynucleotides with a junction oligonucleotide complementary to the 3′ flanking sequence of a first said construction polynucleotide and a 5′ flanking sequence of a second said construction polynucleotide.

In another embodiment, the construction polynucleotides and the junction oligonucleotide form of a Holliday junction and the method further comprises the step of contacting said Holiday junction with a resolvase.

In certain embodiments, the junction oligonucleotide comprises a sequence that is complementary to: (i) at least a portion of the 3′ flanking sequence of one strand of a first construction polynucleotide, (ii) at least a portion of the 5′ flanking sequence of one strand of a second construction polynucleotide, and interposed therebetween, and (iii) at least two base pairs of the 5′ terminal region of the medial segment of the other strand of the first construction polynucleotide and at least two base pairs of the 3′ terminal region of the medial segment of the other strand of the second construction polynucleotide. Alternatively, in certain such embodiments, In certain embodiments, the junction oligonucleotide comprises a sequence that is complementary to: (i) at least a portion of the 3′ flanking sequence of one strand of a first construction polynucleotide, (ii) at least a portion of the 5′ flanking sequence of one strand of a second construction polynucleotide, and interposed therebetween, and (iii) at least 4 inosine residues that bind to at least 2 base pairs of each medial segment of the construction polynucleotides.

In certain embodiments, the junction oligonucleotide comprises a sequence that is complementary to: (i) at least a portion of the 3′ flanking sequence of one strand of a first construction polynucleotide, (ii) at least a portion of the 5′ flanking sequence of one strand of a second construction polynucleotide, and interposed therebetween, and (iii) a mixture of sequences comprising at least 4 consecutive N base pairs, wherein N is A, T, U, G, or C, and wherein at least a portion of the sequences of the mixture of junction oligonucleotide sequences bind to at least 2 base pairs of each medial sequence of the construction polynucleotides.

In certain embodiments, the junction oligonucleotides hybridize to the medial segments of the construction polynucleotides by one or more wobble base pairings.

In certain embodiments, the methods described herein may further comprise creating single stranded overhangs corresponding both to a 3′ flanking region and to a 5′ flanking region in a said construction polynucleotide while retaining its medial segments intact and free of residual flanking sequence bases, contacting the construction polynucleotides under hybridization conditions with at least two said junction oligonucleotides to form a complex wherein the medial segments of the construction polynucleotides are aligned end to end; and exposing the complex to ligation conditions, thereby to form a larger polynucleotide construct comprising at least three fused said medial segments.

In various embodiments, the ligation conditions may be enzymatic ligation conditions or chemical ligation conditions.

In certain embodiments, the left and right flanking terminal double stranded sequences of the construction polynucleotides comprise nested binding sites for two or more primer pairs.

In certain embodiments, the methods described herein may further comprise providing a desired said construction polynucleotide by amplifying it selectively from a construction polynucleotide mixture using combination of two or more said primer pairs.

In certain embodiments, at least five construction polynucleotides may be joined together in a single reaction mixture.

In certain embodiments, at least two, three, four, five, or more, larger polynucleotide construct may be formed in a single reaction mixture.

In certain embodiments, the methods described herein may involve repeating steps wherein the polynucleotide construct from a first round becomes a construction polynucleotide for the next round.

In certain embodiments, the methods may involve joining two or more construction polynucleotides having medial segments that comprise homologous sequences.

In certain embodiments, the methods may utilize one or more polynucleotide constructs having medial segment(s) that comprise an open reading frame or a regulatory sequence.

In certain embodiments, the methods may comprise amplifying the larger polynucleotide construct after step d) using primers complementary to 5′ terminal flanking regions of said larger polynucleotide.

In certain embodiments, one or more construction polynucleotide(s) may be coupled to a solid support. For example, the one or more construction polynucleotide(s) may be coupled to a solid support, for example, by a cleavable linker or by hybridization to an oligonucleotide attached to the support. Exemplary cleavable linkers include, for example, photo-labile linkers. When coupling to a solid support involves hybridization between a construction polynucleotide and an oligonucleotide attached to the support, the oligonucleotide may comprise a sequence that is complementary to at least a portion of a flanking sequence of the construction polynucleotide. In certain embodiments, the oligonucleotide attached to the support is not capable of being ligated to an adjacent polynucleotide.

In another aspect, the invention provides a method of producing a polynucleotide construct by joining together in a preselected order a selected pair of construction polynucleotides, the method comprising: (a) providing a mixture of different candidate construction polynucleotides comprising: a medial segment for joinder with another, flanked by 5′ and 3′ flanking sequences, wherein the flanking sequences comprise nested binding sites for two or more primer pairs; (b) providing a mixture of junction oligonucleotides comprising (i) a sequence that hybridizes to both a 5′ and a 3′ flanking sequence of at least one pair of construction polynucleotides, flanked 5′ and 3′ by (ii) junction oligonucleotide flanking sequences comprising binding sites for at least one pair of primers, thereby to enable amplification of a said junction oligonucleotide; (c) providing a plurality of primer pairs; (d) selecting at least a pair of construction polynucleotides from said mixture of candidate construction polynucleotides by amplification thereof with one or more of the primer pairs; (e) selecting a junction oligonucleotide from said mixture of junction oligonucleotides by amplification thereof with one or more of the primer pairs; (f) forming single stranded overhangs on the selected pair of construction polynucleotides thereby to produce a 3′ single stranded overhang corresponding to at least a portion of the 3′ flanking region of a first construction polynucleotide and a 5′ single stranded overhang corresponding to at least a portion of the 5′ flanking region of a second construction polynucleotide; (g) contacting the construction polynucleotide pair with their respective junction oligonucleotide under hybridization conditions to form a complex wherein the junction oligonucleotide is hybridized to the single stranded overhangs of the construction polynucleotides and the medial segments are aligned end to end; and (h) exposing the complex to ligation conditions, thereby to form a larger polynucleotide construct comprising fused said medial segments.

In certain embodiments, the method may further comprise removing the 5′ and 3′ flanking regions from the junction oligonucleotide.

In certain embodiments, the complex forms a Holliday junction. In such embodiments, the method may further comprise contacting the complex with a resolvase.

In certain embodiments, the method may further comprising amplifying the larger polynucleotide construct.

In another aspect, the invention provides a method of preparing a polynucleotide construct, the method comprising the steps of: (a) providing a plurality of double stranded construction polynucleotides each comprising at least a sense strand consisting of a sequence to be incorporated within said polynucleotide construct; (b) providing a plurality of different junction oligonucleotides defining high fidelity sequences complementary to respective 3′ and 5′ termini of construction polynucleotides to be joined by chemically synthesizing in parallel on a surface, severing from the surface, and purifying a plurality of said junction oligonucleotides; (c) mixing construction polynucleotides and junction oligonucleotides together under hybridizing conditions to produce an intermediate comprising serially arranged construction polynucleotides linked by bridging hybridized junction oligonucleotides, and (d) subjecting the mixture to a ligase and/or to a polymerase and dNTPs to prepare said polynucleotide construct.

In another aspect, the invention provides a method of preparing a polynucleotide construct, the method comprising the steps of: (a) providing a plurality of different double stranded construction polynucleotides comprising at least a sense strand consisting of a sequence to be incorporated within said polynucleotide construct; (b) extracting by amplification from a reservoir of a plurality of junction oligonucleotides a set of different selected junction oligonucleotides defining high fidelity sequences complementary to respective 3′ and 5′ termini of construction polynucleotides to be joined; (c) mixing construction polynucleotides and said selected junction oligonucleotides together under hybridizing conditions to produce an intermediate comprising serially arranged construction polynucleotides linked by bridging junction oligonucleotides, and (d) subjecting the mixture to a ligase and/or to a polymerase and dNTPs to prepare said polynucleotide construct.

In certain embodiments, amplification may be conducted by PCR using primers which anneal to primer hybridization sites flanking said junction oligonucleotides.

In certain embodiments, the methods further comprise the additional step of restricting said primer hybridization sites from said junction oligonucleotides prior to step (c).

In certain embodiments, said polynucleotide construct comprises more than 1 Kb, 5 Kb, 10 Kb or 100 Kb.

In certain embodiments, the methods may comprise preparing a plurality of different polynucleotide constructs simultaneously by providing in step (a) first and second said pluralities of double stranded construction polynucleotides and providing in step (b) first and second different sets of junction oligonucleotides.

In certain embodiments, the methods described herein may comprise producing a plurality of different polynucleotide candidates for expression and testing for a preselected property. In certain such embodiments, said plurality of different polynucleotide candidates may comprise DNAs encoding (i) different sequences in a single open reading frame or (ii) a plurality of candidate metabolic pathways encoding enzymes and regulatory elements thereof.

In certain embodiments, said plurality of double stranded construction polynucleotides are blunt ended polynucleotides.

In certain embodiments, said plurality of double stranded construction polynucleotides are provided by selective amplification thereof from a mixture of candidate construction polynucleotides using selectively removable primer sites on said construction polynucleotides.

In certain embodiments, the methods may further comprises chemically synthesizing in parallel on a surface said different junction oligonucleotides using removable primer sites common to a plurality of said different junction oligonucleotides, and amplifying plural said junction oligonucleotides using a single pair of primers.

In another aspect, the invention provides a set of construction polynucleotides comprising a plurality of members adapted for connection to one another in a selected order to produce a larger polynucleotide, plural members of the set comprising (i) a medial segment comprising a DNA sequence for inclusion within a said larger polynucleotide, and (ii) left and right flanking terminal double stranded sequences of a length sufficient to permit selective hybridization of a complementary DNA thereto, plural of the members being designed to enable selective purposeful creation of a single stranded overhang corresponding to a sequence of a said left flanking region, a said right flanking region, or both said regions, while the medial segment remains intact and free of flanking sequence bases, thereby to enable directed scarless ligation of any one member to any other.

In various embodiments, the set may comprise at least three, five, ten, or more members.

In certain embodiments, plural members of the set are designed to create selectively a said single stranded overhang therewithin using a protocol orthogonal to another said member thereby to permit multiplexed connection of two or more selected members in the presence of other members.

In certain embodiments, at least one of said flanking sequences of at least one member of the set comprises a uracil residue at the junction of said flanking region and said medial segment.

In certain embodiments, at least one of said flanking sequences, optionally together with a portion of said medial segment, of at least one member of the set defines a recognition sequence for a type IIS restriction endonuclease so that said restriction endonuclease cleaves said flanking sequence at the junction thereof with said medial segment.

In certain embodiments, at least one member of the set comprises a bulky group positioned at the junction between said medial segment and said left or right flanking sequence, or both flanking sequences, and wherein said bulky group blocks removal of nucleotides therebeyond by a 3′ to 5′ or a 5′ to 3′ exonuclease leaving said medial segment and optionally the other flanking sequence intact.

In certain embodiments, at least one member of the set comprises a medial segment having a selected 3′ terminating nucleobase X consisting of A, G, T, C, or U and a 3′ flanking sequence free of a said nucleobase X so as to permit removal of said 3′ flanking sequence while maintaining said medial segment intact by the action of a 3′ to 5′ exonuclease activity of a polymerase in the presence of DNTP-X.

In certain embodiments, at least a portion of the flanking sequence on the 3′ end of at least one strand of plural said members of the set has the same sequence.

In certain embodiments, at least a portion of the flanking sequence on the 5′ end of at least one strand of plural said members of the set has the same sequence.

In certain embodiments, the set may further comprise one or a plurality of junction oligonucleotides comprising a sequence that hybridizes to the 3′ terminal regions of the medial segment of one construction polynucleotide and the 5′ terminal region of the medial segments of the same or a different construction polynucleotide.

In certain embodiments, the set may further comprise one or a plurality of junction oligonucleotides comprising a sequence that hybridizes to a 3′ flanking sequence of one construction polynucleotide and a 5′ flanking sequence of the same or a different construction polynucleotide. In certain such embodiments, said junction oligonucleotides may further comprise a medial sequence that is complementary to at least 2 base pairs of the 3′ and 5′ terminal regions of the medial segments of at least one pair of construction polynucleotides.

In certain embodiments, said junction oligonucleotides may further comprise 3′ and 5′ flanking sequences to permit amplification thereof. In certain such embodiments, the 3′ and 5′ flanking sequences of a plurality of said junction oligonucleotides may comprise primer binding sites having the same sequences. In certain embodiments, the 3′ and 5′ flanking sequences of the junction oligonucleotides are removable.

In certain embodiments, a plurality of the construction polynucleotides of the set are mixed together and at least a subset comprise flanking sequences comprising: (i) nested primer pairs which permit selective amplification of one or more construction polynucleotides from said pool or (ii) an affinity sequence which permits selective removal of one or more construction polynucleotides from said pool.

In certain embodiments, the sets described herein may further comprise one or a plurality of primer pairs that bind to 5′ flanking sequences of each strand of one or more construction polynucleotides in the set.

In certain embodiments, said affinity sequence is removable by treatment with an endonuclease while leaving said medial segment or said flanking sequences and said medial segment intact.

In certain embodiments, said polynucleotides are assembled from chemically synthesized oligonucleotides or amplification products thereof.

In certain embodiments, said medial segments of two or members of the set may comprise homologous sequences.

In certain embodiments, said medial segment(s) of one or more members of the set may comprise an open reading frame or a regulatory sequence.

In another aspect, the invention provides a composition of matter comprising a pair of chemically synthesized double stranded construction polynucleotides adapted for connection to one another in a selected order to produce a larger fused polynucleotide, each member of the pair comprising a medial segment comprising a DNA sequence for inclusion within said fused polynucleotide, a first member of the pair comprising a single stranded 3′ overhang flanking sequence outside its said medial segment and a second member of the pair comprising a single stranded 5′ overhang flanking sequence outside its said medial segment.

In certain embodiments, the composition may further comprise a third said double stranded construction polynucleotides adapted for connection to the others in a selected order to produce an at least three-membered fused polynucleotide wherein at least one of said three members comprises overhang flanking sequence outside its said medial segment on both its 5′ and 3′ ends.

In certain embodiments, the composition may further comprise one or more junction oligonucleotides comprising a DNA sequence complementary to a 3′ overhang flanking sequence of one construction polynucleotide and a 5′ overhang flanking sequence of the same or a different construction polynucleotide.

In certain embodiments, the composition may further comprise one or more junction oligonucleotides comprising a DNA sequence complementary to both a 3′ sequence of the medial segment of one construction polynucleotide and a 5′ sequence of the medial segment of the same or a different construction polynucleotide.

In certain embodiments, the 3′ ends of one of the strands of each said construction polynucleotide comprise nested primer binding sites.

In certain embodiments, at least one of the 5′ and 3′ flanking sequences of one or both construction polynucleotides are removable while leaving said medial segments intact and containing no flanking sequence bases.

In certain embodiments, the construction polynucleotides comprise amplification products of chemically synthesized oligonucleotides.

A composition comprising a plurality of different junction oligonucleotides, each respective said junction oligonucleotides comprising, from 5′ to 3′, a nucleotide sequence complementary to a 3′ terminal sequence of one construction polynucleotide, and a nucleotide sequence complementary to a 5′ terminal sequence of another construction polynucleotide, said junction oligonucleotides having a sequence error rate less than about one base in 1000 so as to enable simultaneous selective hybridization of plural said junction oligonucleotides with their respective plural complementary construction polynucleotides and the preparation in parallel of multiple fusions between construction polynucleotides.

In certain embodiments, said junction oligonucleotides further comprise common removable primer binding sites on the ends thereof to permit amplification thereof with a pair of common primers.

In certain embodiments, said different junction oligonucleotides are immobilized on a surface, and are adapted for severance therefrom.

In certain embodiments, said junction oligonucleotides have a sequence error rate less than about one base in 1500, 2000, 3000, 5000, or 10,000 bases.

The appended claims are incorporated into this section by reference.

### BRIEF DESCRIPTION OF THE FIGURES

The foregoing and other features and advantages of the present invention will be more fully understood from the following detailed description of illustrative embodiments taken in conjunction with the accompanying drawings in which:

FIG. 1 shows a junction assembly method for assembling two or more nucleic acids in a desired order. Panel A shows the polynucleotide construct desired to be produced (e.g., CADB). Panel B shows the starting materials that may be used to produce the polynucleotide construct shown in panel A, including construction polynucleotides (A, B, C and D), junction oligonucleotides (C_{C}C_{A}, C_{A}C_{D} and C_{D}C_{B}), and primers C_{F} and B_{R}. Panel C shows an exemplary product of mixing together the polynucleotide constructs. Panel D shows one method for connecting the construction polynucleotides together using ligase. Panel E shows an alternative method for connecting the construction polynucleotides together using ligase, polymerase and dNTPs. The dotted lines represent areas that have been extended by polymerase and the arrows show the direction of chain extension.

FIG. 2 shows a junction assembly method for joining two nucleic acids in a desired order wherein both strands of one flanking sequence on each construction polynucleotide are removed before joining. The method utilizes ajunction oligonucleotide having a sequence specific for the medial segments of the construction polynucleotides to be joined. The starting materials are designated as follows: L_{T} and L_{B} represent the top and bottom strands of the medial segment of the construction polynucleotide to be joined on the left (construction polynucleotide L), FP_{L} and RP_{L} represent the forward and reverse primer binding sites, respectively, for construction polynucleotide L, R_{T} and R_{B} represent the top and bottom strands of the medial segment of the construction polynucleotide to be joined on the right (construction polynucleotide R), FP_{R} and RP_{R} represent the forward and reverse primer binding sites, respectively, for construction polynucleotide R, and C_{L}C_{R} represents the junction oligonucleotide wherein C_{L} represents a sequence complementary to the 3′ portion of the medial segment of construction polynucleotide L and C_{R} represents a sequence complementary to the 5′ portion of the medial segment of construction polynucleotide R. The desired product is a joining of the 3′ end of construction polynucleotide L with the 5′ end of construction polynucleotide R and excluding any sequence from the RP_{L} and FP_{R} flanking regions.

FIG. 3 shows a junction assembly method for joining two nucleic acids in a desired order wherein each construction polynucleotide has a single stranded overhang on the end to be joined. The single stranded overhangs can be generated using various specific techniques disclosed herein, e.g., by exposing a specific type ila restriction endonuclease to a construction polynucleotides specially designed to have a sequence recognized specifically by the endonuclease which cuts at the junction between the medial segment and the primer site. The method utilizes a junction oligonucleotide having a sequence specific for the medial segments of the construction polynucleotides to be joined. L_{T}, L_{B}, RP_{L}, RP_{L}, R_{T}, R_{B}, FP_{R}, RP_{R}, C_{L} and C_{R} and the desired product are as described above for FIG. 2.

FIG. 4 shows a junction assembly method for joining two nucleic acids in a desired order wherein one of the strands of each construction polynucleotide is removed from the reaction before joining. The method utilizes ajunction oligonucleotide having a sequence specific for the medial segments of the construction polynucleotides to be joined. L_{T}, L_{B}, RP_{L}, RP_{L}, R_{T}, R_{B}, FP_{R}, RP_{R}, C_{L} and C_{R} and the desired product are as described above for FIG. 2.

FIG. 5 shows a junction assembly method for joining two nucleic acids in a desired order using a Holliday junction and a resolvase protein. The method utilizes a junction oligonucleotide comprising sequences complementary to one flanking region of each construction polynucleotide and is not dependent on the sequence of the medial segment of the construction polynucleotides. L_{T}, L_{B}, RP_{L}, RP_{L}, R_{T}, R_{B}, FP_{R}, and RP_{R} and the desired product are as described above for FIG. 2. J_{L}J_{R} represents the junction oligonucleotide wherein J_{L} represents a sequence complementary to the 3′ flanking region of construction polynucleotide L and J_{R} represents a sequence complementary to the 5′ flanking region of construction polynucleotide R.

FIG. 6 shows two variations of the junction oligonucleotides that can be used in association with the junction assembly methods illustrated in FIGS. 5 or 7. The alternative junction oligonucleotides are useful when joining two construction polynucleotides having short single stranded overhangs. FIG. 6A shows a junction oligonucleotide that is self complementary at the ends thereby forming hairpins that may be ligated to the single stranded overhangs of the construction polynucleotides. FIG. 6B shows a junction oligonucleotide used in conjunction with two adapter sequences (A_{L} and A_{R}) that are complementary to the 5′ portion of J_{L} and the 3′ portion of J_{R}, respectively.

FIG. 7 shows a junction assembly method for joining two nucleic acids in a desired order using a junction oligonucleotide to form a bridge. The method utilizes a junction oligonucleotide comprising sequences complementary to one flanking region of each construction polynucleotide and is not dependent on the sequence of the medial segment of the construction polynucleotides. L_{T}, L_{B}, RP_{L}, RP_{L}, R_{T}, R_{B}, FP_{R}, and RP_{R} and the desired product are as described above for FIG. 2. J_{L}-N-J_{R} represents the junction oligonucleotide wherein J_{L} and J_{R} are as described above for FIG. 4 and N represents a sequence of 4 to 8 universal bases (e.g., inosine or 5-nitroindole) or 4-8 degenerate bases (e.g., A, G, T, or C at each location; the junction oligonucleotide is a pool, e.g., a pool of 4⁴ or 256 oligonucleotides when N is four bases in length).

FIG. 8 illustrates an example of a junction assembly method used to join together two branched nucleic acid structures. The directionality of one of the branched structures is shown for purposes of illustration.

FIG. 9 illustrates two variations of hierarchical assembly reactions.

FIG. 10 illustrates a hierarchical assembly method that involves multiplex assembly in at least one reaction pool.

FIG. 11 illustrates a variety of methods for producing a single stranded overhang of a desired length at the 3′ end of a double stranded polynucleotide.

FIG. 12 illustrates a variety of methods for producing a single stranded overhang of a desired length at the 5′ end of a double stranded polynucleotide.

FIG. 13 illustrates an exemplary embodiment of a set of construction polynucleotides, junction oligonucleotides and primer pairs contained in a multi-well plate.

FIG. 14 illustrates a flow diagram of an automated system that may be used to prepare polynucleotide constructs in accordance with the methods described herein.

### DETAILED DESCRIPTION

1. Definitions

As used herein, the following terms and phrases shall have the meanings set forth below. Unless defined otherwise, all technical and scientific terms used herein have the same meaning as commonly understood to one of ordinary skill in the art.

The singular forms “a,” “an,” and “the” include plural reference unless the context clearly dictates otherwise.

The term “amplification” means that the number of copies of a nucleic acid fragment is increased.

The term “AP endonuclease” refers to an endonuclease that recognizes an abasic (e.g., apurinic or apyrimidinic) site in a DNA duplex and removes the ribose-phosphate moiety from the backbone forming a single stranded break. Abasic sites may be formed by DNA glycosylases, such as, for example, Ura-DNA-glycosylase (recognizes uracil bases), thymine-DNA glycosylase (recognizes GIT mismatches), and Mut Y (recognizes G/A mismatches). Exemplary AP endonucleases include, for example, APE 1 (or HAP 1 or Ref-1), Endonuclease III, Endonuclease IV, Endonuclease VIII, Fpg, or Hogg1, all of which are commercially available, for example, from New England Biolabs (Beverly, MA).

The term “base-pairing” refers to the specific hydrogen bonding between purines and pyrimidines in double-stranded nucleic acids including, for example, adenine (A) and thymine (T), guanine (G) and cytosine (C), (A) and uracil (U), and guanine (G) and cytosine (C), and the complements thereof. Base-pairing leads to the formation of a nucleic acid double helix from two complementary single strands.

The term “cleavage” as used herein refers to the breakage of a bond between two nucleotides, such as a phosphodiester bond.

The terms “comprise” and “comprising” are used in the inclusive, open sense, meaning that additional elements may be included.

The term “conserved residue” refers to an amino acid that is a member of a group of amino acids having certain common properties. The term “conservative amino acid substitution” refers to the substitution (conceptually or otherwise) of an amino acid from one such group with a different amino acid from the same group. A functional way to define common properties between individual amino acids is to analyze the normalized frequencies of amino acid changes between corresponding proteins of homologous organisms (Schulz, G. E. and R. H. Schirmer., Principles of Protein Structure, Springer-Verlag). According to such analyses, groups of amino acids may be defined where amino acids within a group exchange preferentially with each other, and therefore resemble each other most in their impact on the overall protein structure (Schulz, G. E. and R. H. Schirmer, Principles of Protein Structure, Springer-Verlag). One example of a set of amino acid groups defined in this manner include: (i) a charged group, consisting of Glu and Asp, Lys, Arg and His, (ii) a positively-charged group, consisting of Lys, Arg and His, (iii) a negatively-charged group, consisting of Glu and Asp, (iv) an aromatic group, consisting of Phe, Tyr and Trp, (v) a nitrogen ring group, consisting of His and Trp, (vi) a large aliphatic nonpolar group, consisting of Val, Leu and ile, (vii) a slightly-polar group, consisting of Met and Cys, (viii) a small-residue group, consisting of Ser, Thr, Asp, Asn, Gly, Ala, Glu, Gln and Pro, (ix) an aliphatic group consisting of Val, Leu, ile, Met and Cys, and (x) a small hydroxyl group consisting of Ser and Thr.

The term “construction polynucleotide” refers to a single or double stranded polynucleotide that may be used for assembling nucleic acid molecules that are longer than the construction polynucleotide itself. In exemplary embodiments, a construction polynucleotide may be used for assembling a nucleic acid molecule by one or more of the junction assembly methods described herein. Typically a construction polynucleotide is double stranded and may comprise a medial segment surrounded by left and right flanking regions. The medial segment comprises a nucleic acid sequence that is desired to be included in a larger nucleic acid construct (and its complement) and embodies its “information content.” The flanking regions contain sequences that are useful for amplification, manipulation, joinder, and/or isolation of the construction polynucleotide. Such flanking regions may be universal tags and/or binding sites for one or more universal primers. The flanking regions preferably are removable, for example, by enzymatic or chemical methods (e.g., restriction endonuclease cleavage, exonuclease digestion, UDG/AP endonuclease cleavage, etc.). The medial segments of the construction polynucleotides typically comprise specially designed sequences intended to be candidates for assembly with others to produce any one of a number of larger polynucleotides of predefined sequence, for example, exons for assembly combinatorially to encode naturally occurring or novel proteins, or genes or regulatory constructs for ligation into multi-component genetic assemblies. In exemplary embodiments, the medial segment of a construction polynucleotide may have a nucleotide base length from about 400 to about 5000, about 100 to about 2000, about 50 to about 1500, or about 25 to about 500, and any flanking regions may each be from about 5 to about 200, about 10 to about 150, about 25 to about 100, about 25 to about 75, or about 25 to about 50 nucleotides in length. Construction polynucleotides of various lengths, information content, and designs advantageously may be synthesized in parallel as disclosed more fully below and, for example, in U.S. application Ser. Nos. 11/068,321 and 11/067,812 and U.S. Provisional Application No. 60/657,014, all filed on Feb. 28, 2005.

The terms “denature” or “melt” refer to a process by which strands of a duplex nucleic acid molecule are separated into single stranded molecules. Methods of denaturation include, for example, thermal denaturation and alkaline denaturation.

The term “detectable marker” refers to a polynucleotide sequence that facilitates the identification of a cell harboring the polynucleotide sequence. In certain embodiments, the detectable marker encodes for a cheriluminescent or fluorescent protein, such as, for example, green fluorescent protein (GFP), enhanced green fluorescent protein (EGFP), Renilla Reniformis green fluorescent protein, GFPmut2, GFPuv4, enhanced yellow fluorescent protein (EYFP), enhanced cyan fluorescent protein (ECFP), enhanced blue fluorescent protein (EBFP), citrine and red fluorescent protein from discosoma (dsRED). In other embodiments, the detectable marker may be an antigenic or affinity tag such as, for example, a polyHis tag, myc, HA, GST, protein A, protein G, calmodulin-binding peptide, thioredoxin, maltose-binding protein, poly arginine, poly His-Asp, FLAG, etc.

The term “duplex” refers to a nucleic acid molecule that is at least partially double stranded. A “stable duplex” refers to a duplex that is relatively more likely to remain hybridized to a complementary sequence under a given set of hybridization conditions. In an exemplary embodiment, a stable duplex refers to a duplex that does not contain a base pair mismatch, insertion, or deletion. An “unstable duplex” refers to a duplex that is relatively less likely to remain hybridized to a complementary sequence under a given set of hybridization conditions. In an exemplary embodiment, an unstable duplex refers to a duplex that contains at least one base pair mismatch, insertion, or deletion.

The term “gene” refers to a nucleic acid comprising an open reading frame encoding a polypeptide having exon sequences and optionally intron sequences. The term “intron” refers to a DNA sequence present in a given gene which is not translated into protein and is generally found between exons.

The term “hybridize” or “hybridization” refers to specific binding between two complementary nucleic acid strands. In various embodiments, hybridization refers to an association between two perfectly matched complementary regions of nucleic acid strands as well as binding between two nucleic acid strands that contain one or more mismatches (including mismatches, insertion, or deletions) in the complementary regions. Hybridization may occur, for example, between two complementary nucleic acid strands that contain 1, 2, 3, 4, 5, or more mismatches. In various embodiments, hybridization may occur, for example, between complementary strands of a construction polynucleotide, between complementary portions of construction polynucleotides and junction oligonucleotides, between a primer and a primer binding site, etc. The stability of hybridization between two nucleic acid strands may be controlled by varying the hybridization conditions and/or wash conditions, including for example, temperature and/or salt concentration. For example, the stringency of the hybridization conditions may be increased so as to achieve more selective hybridization, e.g., as the stringency of the hybridization conditions are increased the stability of binding between two nucleic acid strands, particularly strands containing mismatches, will be decreased.

The term “including” is used to mean “including but not limited to” “including” and “including but not limited to” are used interchangeably.

The term “junction oligonucleotide” refers to an oligonucleotide that facilitates the joining of two construction polynucleotides. In certain embodiments, junction oligonucleotides comprise a sequence that is complementary to a portion of a first construction polynucleotide and a sequence that is complementary to a portion of a second construction polynucleotide (when forming tandem repeats, the first and second construction polynucleotides may be the same). For example, a junction oligonucleotide may be complementary to at least about 3, 4, 5, 6, 7, 8, 9, 10, 12, 15, 18, 20, 25, or more, consecutive bases of a first construction polynucleotide and at least about 3, 4, 5, 6, 7, 8, 9, 10, 12, 15, 18, 20, 25, or more, consecutive bases of a second construction polynucleotide. The junction oligonucleotide may be complementary to a portion of the medial region or a portion of a flanking region of a construction polynucleotide. In an exemplary embodiment, a junction oligonucleotide comprises a sequence that is complementary to at least a portion of a 3′ flanking region of a first construction polynucleotide and a sequence that is complementary to at least a portion of a 5′ flanking region of a second construction polynucleotide (when forming tandem repeats, the first and second construction polynucleotides may be the same). Junction oligonucleotides may themselves additionally comprise 5′ and 3′ flanking regions that permit amplification and/or isolation of a junction oligonucleotide. Such flanking regions may be universal tags and/or binding sites for one or more universal primers. The flanking regions may optionally be removable, for example, by enzymatic or chemical methods (e.g., restriction endonuclease cleavage, exonuclease digestion, UDG/AP endonuclease cleavage, etc.). Junction oligonucleotides may be single stranded or double stranded.

The term “ligase” refers to a class of enzymes and their functions in forming a phosphodiester bond in adjacent oligonucleotides which are annealed to the same oligonucleotide. Particularly efficient ligation takes place when the terminal phosphate of one oligonucleotide and the terminal hydroxyl group of an adjacent second oligonucleotide are annealed together across from their complementary sequences within a double helix, i.e. where the ligation process ligates a “nick” at a ligatable nick site and creates a complementary duplex (Blackburn, M. and Gait, M. (1996) in Nucleic Acids in Chemistry and Biology, Oxford University Press, Oxford, pp. 132-33, 481-2). The site between the adjacent polynucleotides is referred to as the “ligatable nick site”, “nick site”, or “nick”, whereby the phosphodiester bond is non-existent, or cleaved.

The term “ligate” refers to the reaction of covalently joining adjacent oligonucleotides through formation of an internucleotide linkage.

The term “mutM” refers to an 8-oxoguanine DNA glycosylase that removes 7,8-dihydro-8-oxoguanine (8-oxoG) and formamido pyrimidine (Fapy) lesions from DNA. Exemplary mutM proteins include, for example, polypeptides encoded by nucleic acids having the following GenBank accession Nos. AF148219 (Nostoc PCC8009), AF026468 (Streptococcus mutans), AF093820 (Mastigocladus laminosus), AB010690 (Arabidopsis thaliana), U40620 (Streptococcus mutans), AB008520 (Thermus thermophilus) and AF026691 (Homo sapiens), as well as homologs, orthologs, paralogs, variants, or fragments thereof.

The term “mutY” refers to an adenine glycosylase that is involved in the repair of 7,8-dihydro-8-oxo-2′-deoxyguanosine (OG):A and G:A mispairs in DNA. Exemplary mutY proteins include, for example, polypeptides encoded by nucleic acids having the following GenBank accession Nos. AF121797 (Streptomyces), U63329 (Human), AA409965 (Mus musculus) and AF056199 (Streptomyces), as well as homologs, orthologs, paralogs, variants, or fragments thereof.

The terms “nucleic acid” or “polynucleotide” refer to a polymeric form of nucleotides, either ribonucleotides and/or deoxyribonucleotides or a modified form of either type of nucleotide. The terms should also be understood to include, as equivalents, analogs of either RNA or DNA made from nucleotide analogs, and, as applicable to the embodiment being described, single-stranded (such as sense or antisense) and double-stranded polynucleotides.

The term “oligonucleotide” refers to a short nucleic acid molecule, e.g., a nucleic acid molecule having from about 10 to about 200 nucleotides. Junction oligonucleotides typically are on the order of 20 to 50 bases long. Oligonucleotides may be single stranded or double stranded.

The term “operably linked”, when describing the relationship between two nucleic acid regions, refers to a juxtaposition wherein the regions are in a relationship permitting them to function in their intended manner. For example, a control sequence “operably linked” to a coding sequence is ligated in such a way that expression of the coding sequence is achieved under conditions compatible with the control sequences, such as when the appropriate molecules (e.g., inducers and polymerases) are bound to the control or regulatory sequence(s).

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

The term “polynucleotide construct” refers to a long nucleic acid molecule having a predetermined sequence. Polynucleotide constructs may be assembled from a set of construction polynucleotides and/or a set of subassemblies.

The term “restriction endonuclease recognition site” refers to a nucleic acid sequence capable of binding one or more restriction endonucleases. The term “restriction endonuclease cleavage site” refers to a nucleic acid sequence that is cleaved by one or more restriction endonucleases. For a given enzyme, the restriction endonuclease recognition and cleavage sites may the same or different. Restriction enzymes include, but are not limited to, type I enzymes, type II enzymes, type H1S enzymes, type im enzymes and type IV enzymes. The REBASE database provides a comprehensive database of information about restriction enzymes, DNA methyltransferases and related proteins involved in restriction-modification. It contains both published and unpublished work with information about restriction endonuclease recognition sites and restriction endonuclease cleavage sites, isoschizomers, commercial availability, crystal and sequence data (see Roberts R J et al. (2005) REBASE—restriction enzymes and DNA methyltransferases. Nucleic Acids Res.; 33 Database Issue:D230-2).

The term “selectable marker” refers to a polynucleotide sequence encoding a gene product that alters the ability of a cell harboring the polynucleotide sequence to grow or survive in a given growth environment relative to a similar cell lacking the selectable marker. Such a marker may be a positive or negative selectable marker. For example, a positive selectable marker (e.g., an antibiotic resistance or auxotrophic growth gene) encodes a product that confers growth or survival abilities in selective medium (e.g., containing an antibiotic or lacking an essential nutrient). A negative selectable marker, in contrast, prevents polynucleotide-harboring cells from growing in negative selection medium, when compared to cells not harboring the polynucleotide. A selectable marker may confer both positive and negative selectability, depending upon the medium used to grow the cell. The use of selectable markers in prokaryotic and eukaryotic cells is well known by those of skill in the art. Suitable positive selection markers include, e.g., neomycin, kanamycin, hyg, hisD, gpt, bleomycin, tetracycline, hprt SacB, beta-lactamase, ura3, ampicillin, carbenicillin, chloramphenicol, streptamycin, gentamycin, phleomycin, and nalidixic acid. Suitable negative selection markers include, e.g., hsv-tk, hprt, gpt, and cytosine deaminase.

The term “sequence homology” refers to the proportion of base matches between two nucleic acid sequences or the proportion of amino acid matches between two amino acid sequences. When sequence homology is expressed as a percentage, e.g., 50%, the percentage denotes the proportion of matches over the length of a desired sequence as compared to another sequence. Gaps (in either of the two sequences) are permitted to maximize matching; gap lengths of 15 bases or less are usually used, 6 bases or less are used more frequently, with 2 bases or less used even more frequently. The term “sequence identity” means that sequences are identical (i.e., on a nucleotide-by-nucleotide basis for nucleic acids or amino acid-by-amino acid basis for polypeptides) over a window of comparison. The term “percentage of sequence identity” is calculated by comparing two optimally aligned sequences over the comparison window, determining the number of positions at which the identical amino acids occurs in both sequences to yield the number of matched positions, dividing the number of matched positions by the total number of positions in the comparison window, and multiplying the result by 100 to yield the percentage of sequence identity. Methods to calculate sequence identity are known to those of skill in the art and described in further detail below.

The terms “stringent conditions” or “stringent hybridization conditions” refer to conditions which promote specific hybridization between two complementary polynucleotide strands so as to form a duplex. Stringent conditions may be selected to be about 5° C. lower than the thermal melting point (Tm) for a given polynucleotide duplex at a defined ionic strength and pH. The length of the complementary polynucleotide strands and their GC content will determine the Tm of the duplex, and thus the hybridization conditions necessary for obtaining a desired specificity of hybridization. The Tm is the temperature (under defined ionic strength and pH) at which 50% of a polynucleotide sequence hybridizes to a perfectly matched complementary strand. In certain cases it may be desirable to increase the stringency of the hybridization conditions to be about equal to the Tm for a particular duplex.

A variety of techniques for estimating the Tm are available. Typically, G-C base pairs in a duplex are estimated to contribute about 3° C. to the Tm, while A-T base pairs are estimated to contribute about 2° C., up to a theoretical maximum of about 80-100° C. However, more sophisticated models of Tm are available in which G-C stacking interactions, solvent effects, the desired assay temperature and the like are taken into account. For example, probes can be designed to have a dissociation temperature (Td) of approximately 60° C., using the formula: Td=(((((3×#GC)+(2×#AT))×37)−562)/#bp)−5; where #GC, #AT, and #bp are the number of guanine-cytosine base pairs, the number of adenine-thymine base pairs, and the number of total base pairs, respectively, involved in the formation of the duplex. Other methods for calculating Tm are described in Santal.ucia and Hicks, Annu. Rev. Biomol. Struct. 33: 415-40 (2004) using the formula Tm=ΔH°×1000/(ΔS°+R×ln(C_{T}/x))−273.15, where C_{T} is the total molar strand concentration, R is the gas constant 1.9872 cal/K-mol, and x equals 4 for nonself-complementary duplexes and equals 1 for self-complementary duplexes.

Hybridization may be carried out in 5×SSC, 4×SSC, 3×SSC, 2×SSC, 1×SSC or 0.2×SSC for at least about 1 hour, 2 hours, 5 hours, 12 hours, or 24 hours. The temperature of the hybridization may be increased to adjust the stringency of the reaction, for example, from about 25° C. (room temperature), to about 45° C., 50° C., 55° C., 60° C., or 65° C. The hybridization reaction may-also include another agent affecting the stringency, for example, hybridization conducted in the presence of 50% formamide increases the stringency of hybridization at a defined temperature. In an exemplary embodiment, Betaine, e.g., about 5 M Betaine, may be added to the hybridization reaction to minimize or eliminate the base pair composition dependence of DNA thermal melting transitions (see e.g., Rees et al., Biochemistry 32: 137-144 (1993)). In another embodiment, low molecular weight amides or low molecule weight sulfones (such as, for example, DMSO, tetramethylene sulfoxide, methyl sec-butyl sulfoxide, etc.) may be added to a hybridization reaction to reduce the melting temperature of sequences rich in GC content (see e.g., Chakarbarti and Schutt, BioTechniques 32: 866-874 (2002)).

The hybridization reaction may be followed by a single wash step, or two or more wash steps, which may be at the same or a different salinity and temperature. For example, the temperature of the wash may be increased to adjust the stringency from about 25° C. (room temperature), to about 45° C., 50° C., 55° C., 60° C., 65° C., or higher. The wash step may be conducted in the presence of a detergent, e.g., 0.1 or 0.2% SDS. For example, hybridization may be followed by two wash steps at 65° C. each for about 20 minutes in 2×SSC, 0.1% SDS, and optionally two additional wash steps at 65° C. each for about 20 minutes in 0.2×SSC, 0.1% SDS.

Exemplary stringent hybridization conditions include overnight hybridization at 65° C. in a solution comprising, or consisting of, 50% formamide, 10×Denhardt (0.2% Ficoll, 0.2% Polyvinylpyrrolidone, 0.2% bovine serum albumin) and 200 μg/ml of denatured carrier DNA, e.g., sheared salmon sperm DNA, followed by two wash steps at 65° C. each for about 20 minutes in 2×SSC, 0.1% SDS, and two wash steps at 65° C. each for about 20 minutes in 0.2×SSC, 0.1% SDS.

Hybridization may consist of hybridizing two nucleic acids in solution, or a nucleic acid in solution to a nucleic acid attached to a solid support, e.g., a filter. When one nucleic acid is on a solid support, a pre-hybridization step may be conducted prior to hybridization. Pre-hybridization may be carried out for at least about 1 hour, 3 hours or 10 hours in the same solution and at the same temperature as the hybridization solution (without the complementary polynucleotide strand).

Appropriate stringency conditions are known to those skilled in the art or may be determined experimentally by the skilled artisan. See, for example, Current Protocols in Molecular Biology, John Wiley & Sons, N.Y. (1989), 6.3.1-12.3.6; Sambrook et al., 1989, Molecular Cloning, A Laboratory Manual, Cold Spring Harbor Press, N.Y; S. Agrawal (ed.) Methods in Molecular Biology, volume 20; Tijssen (1993) Laboratory Techniques in biochemistry and molecular biology-hybridization with nucleic acid probes, e.g., part I chapter 2 “Overview of principles of hybridization and the strategy of nucleic acid probe assays”, Elsevier, New York; Tibanyenda, N. et al., Eur. J. Biochem. 139:19 (1984) and Ebel, S. et al., Biochem. 31:12083 (1992); Rees et al., Biochemistry 32: 137-144 (1993); Chakarbarti and Schutt, BioTechniques 32: 866-874 (2002); and SantaLucia and Hicks, Annu. Rev. Biomol. Struct. 33: 415-40 (2004).

As applied to proteins, the term “substantial identity” means that two sequences, when optimally aligned, such as by the programs GAP or BESTFIT using default gap weights, typically share at least about 70 percent sequence identity, alternatively at least about 80, 85, 90, 95 percent sequence identity or more. For amino acid sequences, amino acid residues that are not identical may differ by conservative amino acid substitutions, which are described above.

The term “synthetic,” as used herein with reference to a nucleic acid molecule, refers to production by in vitro chemical and/or enzymatic synthesis.

The term “TDG” refers to a thymine-DNA glycosylase that recognizes G/T mismatches. An exemplary TDG protein includes, for example, a polypeptide encoded by a nucleic acid having GenBank accession No. AF117602 (Ateles paniscus chamek), as well as homologs, orthologs, paralogs, variants, or fragments thereof.

“Transcriptional regulatory sequence” is a generic term used herein to refer to DNA sequences, such as initiation signals, enhancers, and promoters, which induce or control transcription of protein coding sequences with which they are operable linked. In preferred embodiments, transcription of one of the recombinant genes is under the control of a promoter sequence (or other transcriptional regulatory sequence) which controls the expression of the recombinant gene in a cell-type which expression is intended. It will also be understood that the recombinant gene can be under the control of transcriptional regulatory sequences which are the same or which are different from those sequences which control transcription of the naturally-occurring forms of genes as described herein.

As used herein, the term “transfection” means the introduction of a nucleic acid, e.g., an expression vector, into a recipient cell, and is intended to include commonly used terms such as “infect” with respect to a virus or viral vector. The term “transduction” is generally used herein when the transfection with a nucleic acid is by viral delivery of the nucleic acid. The term “transformation” refers to any method for introducing foreign molecules, such as DNA, into a cell. Lipofection, DEAE-dextran-mediated transfection, microinjection, protoplast fusion, calcium phosphate precipitation, retroviral delivery, electroporation, sonoporation, laser irradiation, magnetofection, natural transformation, and biolistic transformation are just a few of the methods known to those skilled in the art which may be used (reviewed, for example, in Mehier-Humbert and Guy, Advanced Drug Delivery Reviews 57: 733-753 (2005)).

The term “type-IIS restriction endonuclease” refers to a restriction endonuclease having a non-palindromic recognition sequence and a cleavage site that occurs outside of the recognition site (e.g., from 0 to about 20 nucleotides distal to the recognition site). Type iHs restriction endonucleases may create a nick in a double stranded nucleic acid molecule or may create a double stranded break that produces either blunt or sticky ends (e.g., either 5′ or 3′ overhangs). Examples of Type Ils endonucleases include, for example, enzymes that produce a 3′ overhang, such as, for example, Bsr I, Bsm I, BstF5 I, BsrD I, Bts I, Mn I, BciV I, Hph I, Mbo II, Eci I, Acu I, Bpm I, Mme I, BsaX I, Bcg I, Bae I, Bfi I, TspDT I, TspGW I, Taq II, Eco57 I, Eco57M I, Gsu I, Ppi I, and Psr I; enzymes that produce a 5′ overhang such as, for example, BsmA I, Ple I, Fau I, Sap I, BspM I, SfaN I, Hga I, Bvb I, Fok I, BceA I, BsmF I, Ksp632 I, Eco31 I, Esp3 I, Aar I; and enzymes that produce a blunt end, such as, for example, Mly I and Btr I. Type-iUs endonucleases are commercially available and are well known in the art (New England Biolabs, Beverly, Mass.). Information about the recognition sites, cut sites and conditions for digestion using type Ils endonucleases may be found, for example, on the world wide web at neb.com/nebecomm/enzymefindersearchbytypeIIs.asp).

The term “universal tag” refers to a nucleotide sequence that flanks a plurality of polynucleotide sequences on the 5′ and/or 3′ termini, e.g., the tag is common to a plurality of polynucleotides. Universal tags may comprise one or more of the following: a primer hybridization sequence, a restriction enzyme recognition site, a restriction enzyme cut site (or half site, e.g., half of the site is contained in the universal tag and half of the site is contained in the polynucleotide sequence), an aptamer, one or more uracil residues, one or more modified nucleic acid residues, or a label for detection and/or immobilization (e.g., biotin, fluorescein, etc.). In an exemplary embodiment, the universal tags comprise one or more binding sites for universal primers.

The term “universal primers” refers to a set of primers (e.g., a forward and reverse primer) that may be used for chain extension/amplification of a plurality of polynucleotides, e.g., the primers hybridize to sites that are common to a plurality of polynucleotides. For example, universal primers may be used for amplification of at least a portion of the polynucleotides in a single pool, such as, for example, a pool of construction polynucleotides and/or a pool of junction oligonucleotides. In certain embodiments, the universal primers may be temporary primers that may be removed after amplification via enzymatic or chemical cleavage. In other embodiments, the universal primers may comprise a modification that becomes incorporated into the polynucleotide molecules upon chain extension. Exemplary modifications include, for example, a 3′ or 5′ end cap, a label (e.g., fluorescein), a particular base (e.g., uracil), or a tag (e.g., a tag that facilitates immobilization or isolation of the polynucleotide, such as, biotin, etc.).

The term “UDG” refers to a uracil-DNA glycosylase that removes free uracil from single stranded or double stranded DNA containing a uracil. Exemplary UDG proteins include, for example, polypeptides encoded by nucleic acids having the following GenBank accession Nos.: AF174292 (Schizosaccharomyces pombe), AF108378 (Cercopithecine herpesvirus), AF125182 (Homo sapiens), AF125181 (Xenopus laevis), U55041 (Homo sapiens), U55041 (Mus musculus), AF084182 (Guinea pig cytomegalovirus), U31857 (Bovine herpesvirus), AF022391 (Feline herpesvirus), M87499 (Human), J04434 (Bacteriophage PBS2), U13194 (Human herpesvirus 6), L34064 (Gallid herpesvirus 1), U04994 (Gallid herpesvirus 2), L01417 (Rabbit fibroma virus), M25410 (Herpes simplex virus type 2), J04470 (S. cerevisiae), J03725 (E. coli), U02513 (Suid herpesvirus), U02512 (Suid herpesvirus) and L13855 (Pseudorabies virus) as well as homologs, orthologs, paralogs, variants, or fragments thereof.

A “vector” is a self-replicating nucleic acid molecule that transfers an inserted nucleic acid molecule into and/or between host cells. The term includes vectors that function primarily for insertion of a nucleic acid molecule into a cell, replication of vectors that function primarily for the replication of nucleic acid, and expression vectors that function for transcription and/or translation of the DNA or RNA. Also included are vectors that provide more than one of the above functions. As used herein, “expression vectors” are defined as polynucleotides which, when introduced into an appropriate host cell, can be transcribed and translated into a polypeptide(s). An “expression system” usually connotes a suitable host cell comprised of an expression vector that can function to yield a desired expression product.

2. Junction Assembly Methods

In one aspect, the invention provides methods for assembling two or more construction polynucleotides together to form a linear or branched structure. The methods utilize junction oligonucleotides to facilitate the covalent attachment of at least two, and preferably multiple construction polynucleotides simultaneously.

The art has now reached a state of development such that polynucleotides of kilobase length having any sequence and a low sequence error rate can be obtained by synthesis, or on occasion retrieved from natural sources (see e.g., U.S. application Ser. Nos. 11/068,321 and 11/067,812 and U.S. Provisional Application No. 60/657,014, all filed on Feb. 28, 2005, etc, incorporated herein by reference). It is such polynucleotides that are termed “construction polynucleotides” herein, and a major objective of the invention is to provide a set of technologies which enable routine assembly of multiple such polynucleotides into larger polynucleotides so as to facilitate the search for novel proteins, synthetic pathways, nanostructural elements, new and useful engineered cells, and other bioparts.

It will be apparent that DNA sequence space is enormous, and that in order to build and test some meaningful number of possible constructs, assembly methods which are fast, versatile, and easy to execute will be required. There are of course known techniques for ligating DNAs which work well for a given ligation task, but all suffer from one or more drawbacks which make them unsuited and impractical for use on the scale contemplated by the inventors hereof. More specifically, the prior art techniques require the planned or serendipitous existence of sequence within the building blocks defining restriction and/or ligation sites, and also often include at the junction of fused parts unwanted bases that limit the utility of the composite part. Prior art ligation techniques normally require isolation of the DNAs to be joined in a reaction vessel so as to eliminate cross reactions, and therefore must execute one ligation at a time, or at best, possibly a few.

The impracticality of these techniques may be illustrated by way of example. Suppose one seeks to build and test a group of protein variants in search of a novel construct having some optimal set of properties. Suppose also that many theoretically possible constructs have been eliminated computationally, and that, having analyzed the problem through a protein design software program, one has a list of potentially successful candidate sequences. The plan calls for building DNAs encoding all such sequences, expressing them in a test system, and assaying for the properties of interest. The strategy is to make the DNAs by assembling in various combinations smaller, specially designed construction polynucleotides differing in sequence. Suppose also that there are five DNA subparts which can be assembled to encode the candidates, and there are just 10 possible sequences in each subpart. The exercise therefore requires the synthesis of 50 different construction polynucleotides, and this can be done in hours, days or at most weeks with state of the art synthesis techniques. However, because there are five subparts and each can be any one of 10 variants, there are four ligations required for each candidate, and 10⁵ different combinations of subparts. If the ligations work perfectly to attach each part directly to the other scarlessly, and take 15 minutes to execute (wildly optimistic), construction of the test set will take 4×0.25×10⁵ or about 10⁵ man hours. Obviously, this approach cannot be executed absent the commitment to very large levels of funding, time, and manpower.

Multiplexing, the simultaneous connection of multiple parts in the correct order in a singe vessel, holds promise as a solution to this problem. Multiplexing requires directed ligation, that is, exploitation of a technique wherein the structure of the parts to be joined, together with the selection of other reagents (such as linkers) added to the reaction, dictate what part(s) will be ligated to which end of which other part(s).

A family of such ligation techniques is provided herein, and serves to enable the assembly of plural, preferably multiple composite constructs simultaneously from building blocks (construction polynucleotides) specially synthesized for the particular exercise, or retrieved from an inventory of parts. All of the techniques require linkers, or “junction oligonucleotides” which are designed specifically to connect one particular construction polynucleotide to one other, or a relatively small number of others. In the example above, one junction oligonucleotide would be needed to connect each of the ten A parts to each of the ten B parts, for a total of 100 linkers, so that 4×100 or 400 different junction oligonucleotides would be needed. More generally, if one has N parts, and seeks the capacity to join any one part with any other (e.g., were each one of the 50 subparts in the example to be attached to each of the others), N² junction oligonucleotides are needed (or in the example, 2500 junctions). Thus the numbers of connectors needed dwarf the already daunting numbers of different parts.

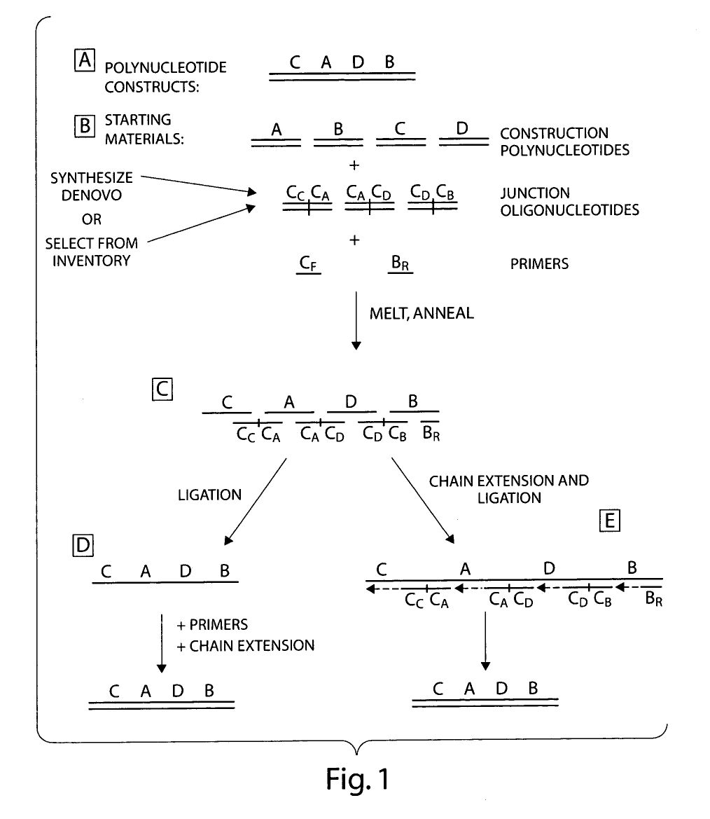

A variety of assembly methods which address this problem are illustrated in FIGS. 1-7. For purposes of illustration only, the construction polynucleotides in FIGS. 1-7 have been designated as left (L) and right (R) sequences having top (T) and bottom (B) strands. It should be understood that the two polynucleotides illustrated in FIGS. 1-7 could be joined in either order (e.g., L-R and R-L). Furthermore, one of skill in the art will understand that nucleic acids have complementary, anti-parallel strands and therefore what is illustrated for the top vs. bottom strands could equally be conducted on the opposite strands (e.g., bottom vs. top).

One solution is to exploit the method of DNA assembly of Mullis et al. (Specific Enzymatic Amplification of DNA In Vitro, Cold Spring Harbor Symposia, p 263, 1986). This involves making one or more large polynucleotides by assembly in parallel of preselected construction polynucleotides using directed parallel ligation mediated by linkers complementary to the 3′ and 5′ ends of segments intended to be joined, in the presence of a ligase and/or a polymerase. Because the trailing and leading terminal base sequence of all the construction polynucleotides are known, and because the order of joinder of the parts of all composite constructs is known, it is possible to synthesize all desired linkers. These serve to direct ligation as they comprise sequence complementary to the 3′ end of one building block and the 5′ end of another. Mixing together all or a portion of the synthesized junction oligonucleotides and the construction polynucleotides to be joined in the presence of a ligase and/or polymerase directs joinder of multiple selected segments to specific others. This method is illustrated in FIG. 1. Panel A shows the polynucleotide construct desired to be produced (e.g., CADB). Panel B shows the starting materials that may be used to produce the polynucleotide construct shown in panel A. The starting materials include construction polynucleotides (A, B, C and D), junction oligonucleotides (C_{C}C_{A}, C_{A}C_{D} and C_{D}C_{B}), and primers C_{F} and B_{R}. As an example, junction oligonucleotide C_{C}C_{A} has a sequence that is complementary to the right terminus of polynucleotide C (e.g., C_{C}) and the left terminus of polynucleotide construct A (e.g., C_{A}). Primers C_{F} and B_{R} represent a forward primer corresponding to the left terminus of polynucleotide C (e.g., C_{F}) and a reverse primer corresponding to the right terminus of polynucleotide B (e.g., B_{R}). The junction oligonucleotides may be synthesized de novo or may be selected from an inventory, including, for example, amplification from a mixture or isolation via an affinity tag as described in more detail below. Panel C shows an exemplary product of mixing together the polynucleotide constructs, the junction oligonucleotides and the primers followed by a round of melting and annealing (the complementary product would also be produced, e.g., with the bottom strands of the construction polynucleotides, the top strands of the junction oligonucleotides, and primer C_{F}). Panel D shows one method for connecting the construction polynucleotides together using ligase. The resulting product (CADB) may then be amplified using the primers C_{F} and B_{R}. Panel E shows an alternative method for connecting the construction polynucleotides together using ligase, polymerase and dNTPs. The dotted lines represent areas that have been extended by polymerase and the arrows show the direction of chain extension. The product (CADB) may optionally be amplified using primers C_{F} and B_{R}. In certain embodiments, the construction polynucleotides and/or junction oligonucleotides may optionally comprise removable flanking sequences that permit amplification and/or isolation of the nucleic acids. Such flanking sequences may be removed prior to assembly.

A key to this technique is the development by the inventors hereof of methods of producing simultaneously multiple high fidelity (low error rate) oligonucleotides each of a pre-specified sequence. This is accomplished by synthesizing on a surface an array of oligonucleotides using methods known per se (see, e.g., PCT Publication No. WO 04/024886; U.S. Pat. Nos. 5,424,186; 5,700,637; 6,083,726; 6,150,102; 6,271,957; 6,375,903; 6,480,324; and U.S. Patent Publication Nos. 2002/0081582; and 2004/0101894), and including temporary primer sites used to amplify the microscopic amounts of the various oligos made on the surface (easily removed sequences outside of and flanking the desired junction oligo sequence, see PCT Publication No. WO 04/024886, U.S. application Ser. Nos. 11/068,321 and 11/067,812 and U.S. Provisional Application No. 60/657,014, all filed on Feb. 28, 2005), and then purifying the synthesized junction oligonucleotides by isolating correct sequences, removing error sequences from the pool, or correcting errors in the copies of the sequences (see U.S. application Ser. Nos. 11/068,321 and 11/067,812 and U.S. Provisional Application No. 60/657,014, all filed on Feb. 28, 2005). This technique admits of various hierarchical and parallel linking strategies, and can be executed quickly and efficiently to produce any one or group of target large polynucleotide constructs in a reasonable time and at a relatively low cost.

Alternatively, rather than producing custom junction oligonucleotides designed specially for a given synthesis task, junction oligonucleotides may be manufactured in advance, maintained in inventory as mixtures in wells or other reservoirs, adapted for retrieval as desired, and tracked by look up tables in a computer or manually. Techniques for storing and enabling selective retrieval of junction oligonucleotides and construction polynucleotides are disclosed below.

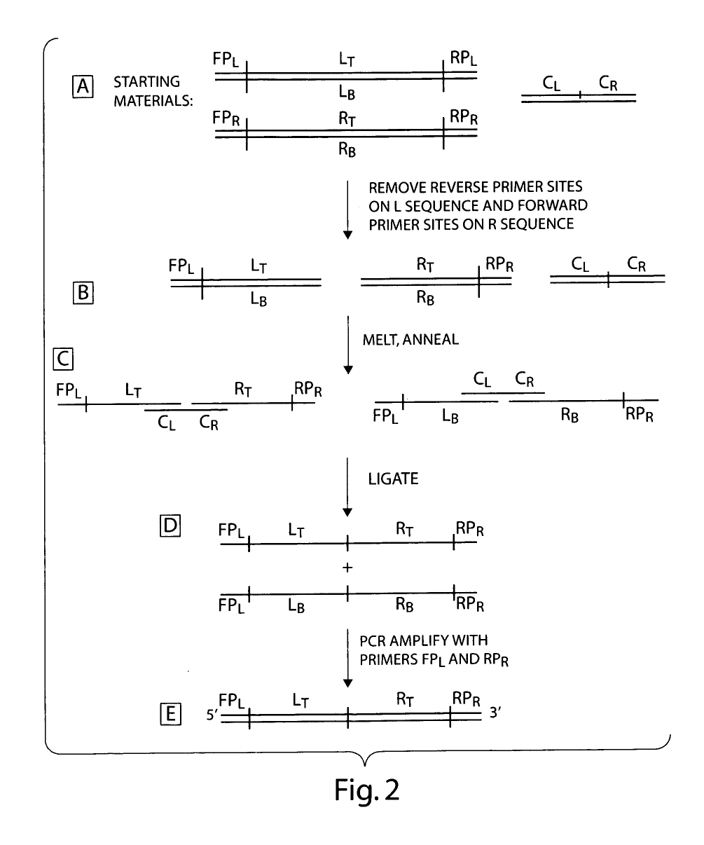

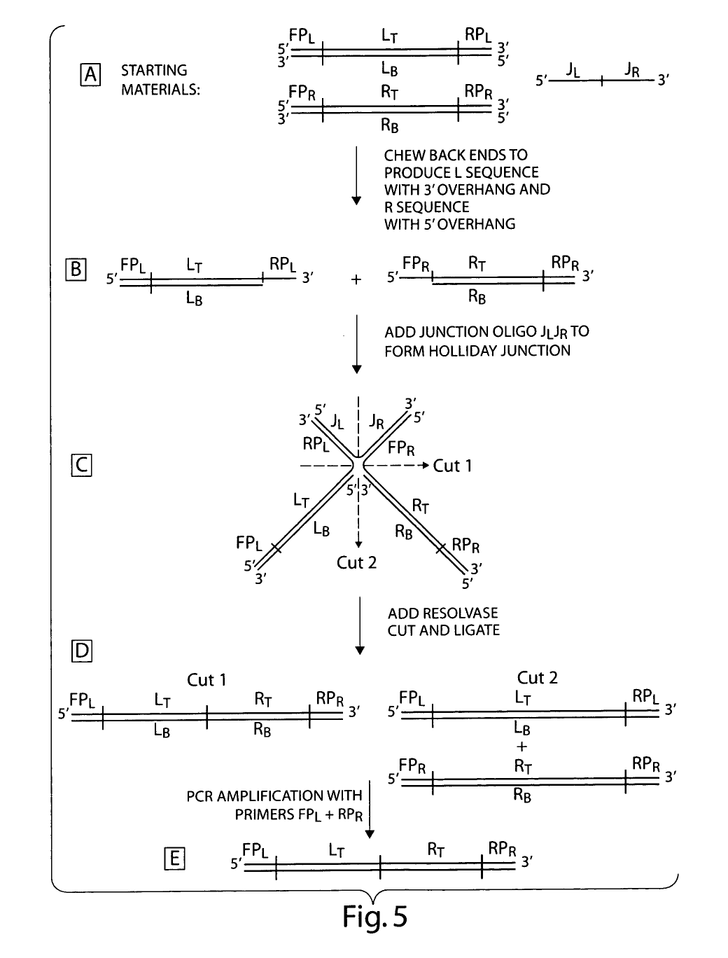

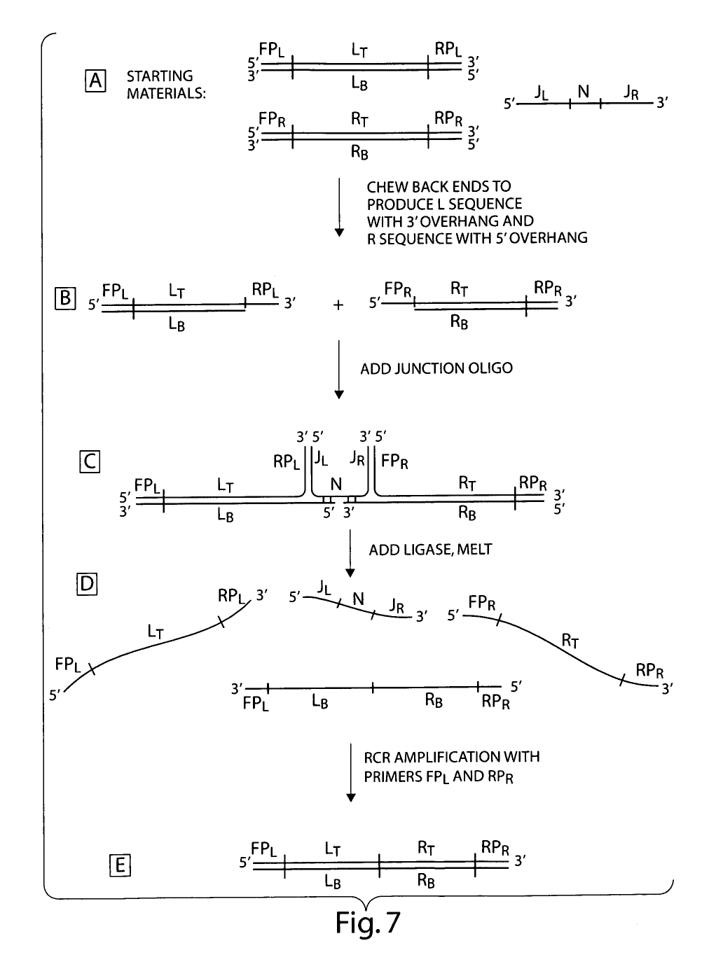

FIG. 2 illustrates one method for joining two polynucleotides in a desired order. As illustrated in FIG. 2A, the starting pool comprises two construction polynucleotides (L and R) and a junction oligonucleotide (C_{L}C_{R}) comprising sequences complementary to the 3′ terminal region of the medial segment of construction polynucleotide L (C_{L}) and the 5′ terminal region of the medial segment of the construction polynucleotide R (C_{R}). The construction polynucleotides contain 5′ and 3′ flanking sequences having primer hybridization sites that permit amplification of the construction polynucleotides, e.g., forward primers left and right (FP_{L} and FP_{R}) and reverse primer left and right (RP_{L} and RP_{R}). The 3′ flanking sequence of the L construction polynucleotide and the 5′ flanking sequence of the R construction polynucleotide may be removed (FIG. 2B) using the methods described below. The strands of the construction polynucleotides and the junction oligonucleotide (if double stranded) are separated. The construction polynucleotides are then contacted with the junction oligonucleotide under hybridization conditions to align the construction polynucleotides side by side based on the complementarity with the junction oligonucleotide (FIG. 2C). The L and R construction polynucleotides may then be covalently joined by ligation forming a polynucleotide comprising the sequences of the L and R construction polynucleotides flanked by 5′ flanking sequence of the L construction polynucleotide and the 3′ flanking sequence of the R construction polynucleotide (FIG. 2D). The polynucleotide may then be amplified using the FP_{L} and RP_{R} primers (FIG. 2E). Amplification with one primer from construction polynucleotide L and one primer from construction polynucleotide R permits confirmation that the correct product has been formed. Any products formed by nonspecific hybridization followed by ligation will not be amplifiable by the primer pair specific for the desired product and will be diluted out of the reaction. After amplification, the remaining flanking sequences may optionally be removed using the methods described below. Alternatively, the flanking sequences may be used in further assembly reactions, e.g., using the methods illustrated in FIGS. 5 and 7 below.

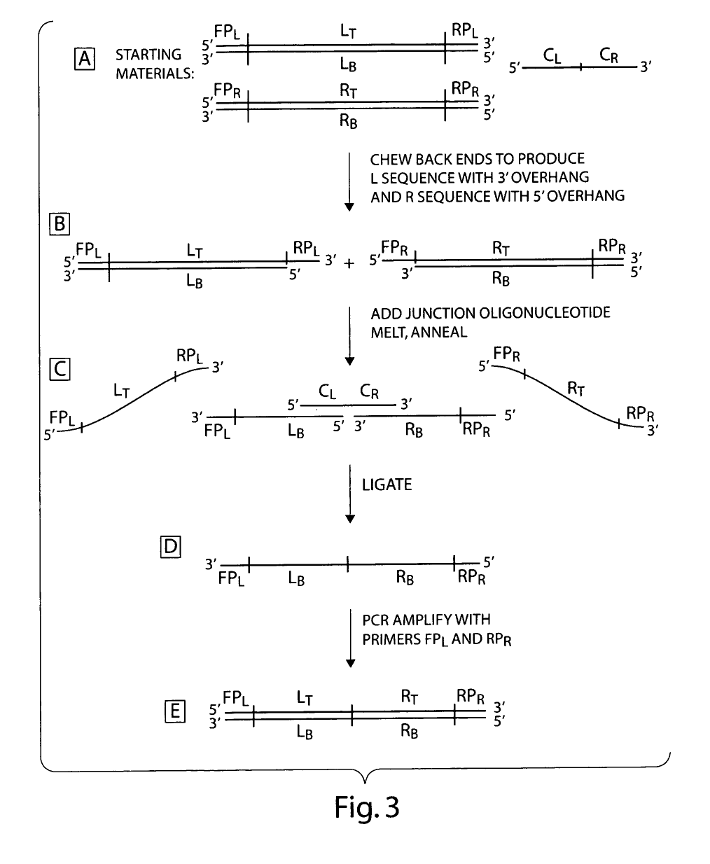

FIG. 3 shows another method for joining any two construction polynucleotides in a desired order. Like FIG. 2, the starting pool comprises two construction polynucleotides (L and R) and a junction oligonucleotide (C_{L}C_{R}) comprising sequences complementary to the 3′ terminal region of the medial segment of construction polynucleotide L (C_{L}) and the 5′ terminal region of the medial segment of the construction polynucleotide R (C_{R}) (FIG. 3A). A portion of the bottom strand of construction polynucleotide L that is complementary to the 3′ flanking region is removed thus producing a partially double stranded polynucleotide having a single stranded 3′ overhang (FIG. 3B). Additionally, a portion of the bottom strand of construction polynucleotide R that is complementary to the 5′ flanking region is removed thus producing a partially double stranded polynucleotide having a single stranded 5′ overhang (FIG. 3B). The strands of the construction polynucleotides and the junction oligonucleotide (if double stranded) are separated. The construction polynucleotides are then contacted with the junction oligonucleotide under hybridization conditions to align the construction polynucleotides end to end based on the complementarity with the junction oligonucleotide (FIG. 3C). The 5′ and 3′ overhangs remaining on the top strands of the construction polynucleotides prevent these strands from being aligned for ligation (FIG. 3C). The reaction mixture may then be exposed to ligation conditions to form a polynucleotide comprising the sequences of the L and R construction polynucleotides flanked by 5′ flanking sequence of the L construction polynucleotide and the 3′ flanking sequence of the R construction polynucleotide (FIG. 3D). The polynucleotide may then be amplified using the FP_{L} and RP_{R} primers (FIG. 3E). Amplification with one primer from construction polynucleotide L and one primer from construction polynucleotide R permits confirmation that the correct product has been formed. Any products formed by nonspecific hybridization followed by ligation will not be amplifiable by the primer pair specific for the desired product and will be diluted out of the reaction. Furthermore, only a very small concentration of the L_{B}-R_{B}/C_{L}-C_{R} duplex need be formed and successfully ligated, as the amplification at best will linearly increase the copy number of L_{T} and R_{T} while geometrically increasing the L_{B}-R_{B} fusion. After amplification, the remaining flanking sequences may optionally be removed using the methods described below. Alternatively, the flanking sequences may be used in further assembly reactions, e.g., using the methods illustrated in FIGS. 5 and 7 below.

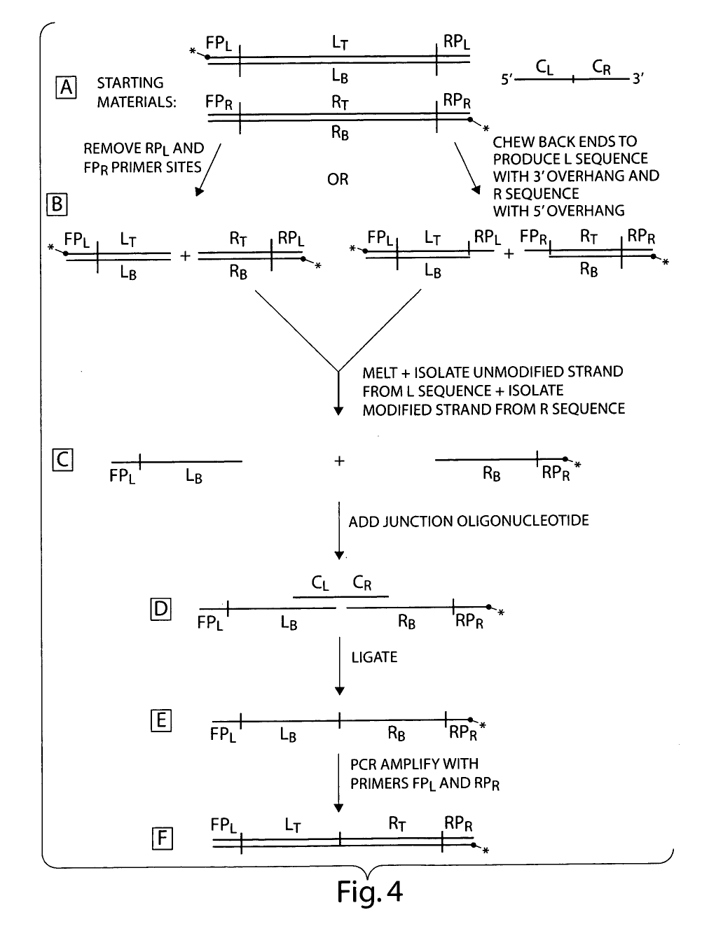

FIG. 4 illustrates yet another method for joining two construction polynucleotides in a desired order. The starting pool comprises two construction polynucleotides (L and R) and a junction oligonucleotide (C_{L}C_{R}) comprising sequences complementary to the 3′ terminal region of the medial segment of construction polynucleotide L (C_{L}) and the 5′ terminal region of the medial segment of the construction polynucleotide R (C_{R}) (FIG. 4A). The 5′ ends of one strand of both construction polynucleotides have been modified with an affinity tag that will permit isolation of one strand of each duplex. The affinity tag may be introduced into the construction polynucleotides using modified primers in a PC_{R} reaction. In an exemplary embodiment, the 5′ ends of the construction polynucleotides may be labeled with biotin to permit strand isolation using avidin (FIG. 4A). As shown in FIGS. 2 and 3, one or both strands of the 3′ flanking region of construction polynucleotide L and the 5′ flanking region of construction polynucleotide R are removed (FIG. 4B). The strands of the construction polynucleotides and the junction oligonucleotide (if double stranded) are separated. The untagged strand of construction polynucleotide L (e.g., L_{B}) and the tagged strand of construction polynucleotide R (e.g., R_{B}) are isolated based on affinity with the tag (e.g., affinity chromatography, etc.) (FIG. 4C). The bottom strands of the construction polynucleotides are then contacted with the junction oligonucleotide under hybridization conditions to align the construction polynucleotides side by side based on the complementarity with the junction oligonucleotide (FIG. 4D). Removal of the top strands of the construction polynucleotides will increase the yield of the joining reaction by decreasing nonproductive associations between the bottom strands of the construction polynucleotides with the top strands of the construction polynucleotides and promoting productive associations between the bottom strands of the construction polynucleotides and the junction oligonucleotides. The reaction mixture may then be exposed to ligation conditions to form a polynucleotide comprising the sequences of the L and R construction polynucleotides flanked by 5′ flanking sequence of the L construction polynucleotide and the 3′ flanking sequence of the R construction polynucleotide (FIG. 4E). The polynucleotide may then be amplified using the FP_{L} and RP_{R} primers (FIG. 4F). Amplification with one primer from construction polynucleotide L and one primer from construction polynucleotide R permits confirmation that the correct product has been formed. Any products formed by nonspecific hybridization followed by ligation will not be amplifiable by the primer pair specific for the desired product and will be diluted out of the reaction. After amplification, the remaining flanking sequences may optionally be removed using the methods described below. Alternatively, the flanking sequences may be used in further assembly reactions, e.g., using the methods illustrated in FIGS. 5 and 7 below. Based on the teachings herein, one of skill in the art would understand that isolation of the opposite strands between steps B and C would be equivalent (e.g., isolated modified strand from construction polynucleotide L and the unmodified strand from construction polynucleotide R).

FIG. 5 illustrates another method for joining two construction polynucleotides in a desired order. The starting pool comprises two construction polynucleotides (L and R) and a junction oligonucleotide (J_{L}J_{R}) comprising sequences complementary to the 3′ flanking region of construction polynucleotide L (J_{L}) and the 5′ flanking region of construction polynucleotide R (J_{R}) (FIG. 5A). A portion of the bottom strand of construction polynucleotide L that is complementary to the 3′ flanking region is removed thus producing a partially double stranded polynucleotide having a single stranded 3′ overhang (FIG. 5B). Additionally, a portion of the bottom strand of construction polynucleotide R that is complementary to the 5′ flanking region is removed thus producing a partially double stranded polynucleotide having a single stranded 5′ overhang (FIG. 5B). The construction polynucleotides are then contacted with the junction oligonucleotide under hybridization conditions in the presence of resolvase to form a Holliday junction (FIG. 5C). The complementarity between the junction oligonucleotide and the flanking regions of the construction polynucleotides will cause the junction to form centered on the location where the medial segment abuts the flanking regions. The resolvase may cut the Holliday junction in two possible configurations. Cut 1 will cleave the top strand of construction polynucleotide L at the junction between the medial segment and the 3′ flanking region and cleave construction polynucleotide R at the junction between the medial segment and the 5′ flanking region (FIG. 5C). Upon exposure to ligation conditions, the top strands of construction polynucleotides L and R will be ligated together and the 3′ flanking region of construction polynucleotide L and the 5′ flanking region of construction polynucleotide R will be removed (FIG. 5D left). Alternatively, the resolvase may cleave the Holliday junction at cut 2 (FIG. 5C). Cut 2 will cleave the junction oligonucleotide between the region complementary to the 3′ flanking region of construction polynucleotide L and the region complementary to the 5′ flanking region of construction polynucleotide R. Upon exposure to ligation conditions, a portion of the junction oligonucleotide will be covalently attached to the bottom strand of the construction polynucleotides thereby reforming construction polynucleotides L and R as pictured in the starting pool (FIG. 5D right). The ligated pool will thus contain a mixture of products formed by cut 1 and cut 2. The desired product comprising the sequences of the L and R construction polynucleotides flanked by S′ flanking sequence of the L construction polynucleotide and the 3′ flanking sequence of the R construction polynucleotide may then be selected by PC_{R} (FIG. 5E). Only the product formed by cut 1 will be amplified with primers FP_{L} and RP_{R} and the products formed by cut 2 will be diluted out of the reaction. After amplification, the remaining flanking sequences may optionally be removed using the methods described below. Alternatively, the flanking sequences may be used in further assembly reactions.

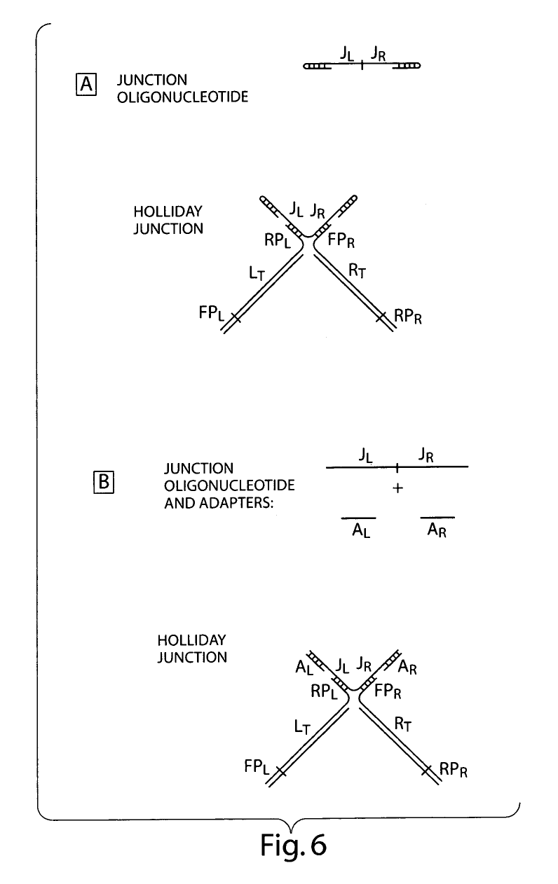

FIG. 6 shows two additional variations on methods useful in various contexts for joining construction polynucleotides having short 3′ and/or 5′ overhangs, e.g., when using a restriction enzyme to produce an overhang having about 2 to about 5 bases. FIG. 6A illustrates formation of a Holliday junction using a junction oligonucleotide having self complementary ends that form hairpins. Upon hybridization with the construction polynucleotides, the folded back ends of the junction oligonucleotide can be ligated to the ends of the construction polynucleotides thereby extending the overhangs. The Holliday junction may then be formed upon addition of a resolvase. FIG. 6B illustrates formation of a Holliday junction using a junction oligonucleotide and adapter oligonucleotides complementary to a portion of the junction oligonucleotides. Upon hybridization with the construction polynucleotides, the adapter oligonucleotides will align with the flanking regions of the construction polynucleotides so that they can be ligated together thereby extending the overhangs. The Holliday junction may then be formed upon addition of a resolvase.

FIG. 7 shows still another method for joining two construction polynucleotides in a desired order. The starting pool comprises two construction polynucleotides (L and R) and a junction oligonucleotide (J_{L}-N-J_{R}) comprising sequences complementary to the 3′ flanking region of construction polynucleotide L (J_{L}) and the 5′ flanking region of construction polynucleotide R (J_{R}) and a center portion (N) (FIG. 7A). The N region of the junction oligonucleotide is a sequence of about 4, 6, or 8 base pairs having a nonspecific sequence. In one embodiment, the N portion of the junction oligonucleotide comprises about 4, 6, or 8 universal bases, such as inosine or 5-nitroindole, that can base pair with A, T, C or G. In an alternative embodiment, the N portion of the junction oligonucleotide comprises about 4, 6, or 8 degenerate bases (e.g., one of A, T, C, G or I at each location). When using degenerate bases, the junction oligonucleotide represents a mixture of oligonucleotides having a variety of sequences in the N region flanked by unchanging sequences in the J regions. For example, when N is 4 degenerate bases, the junction oligonucleotide is a mixture of 4⁴ sequences (or 256). A portion of the bottom strand of construction polynucleotide L that is complementary to the 3′ flanking region is removed thus producing a partially double stranded polynucleotide having a single stranded 3′ overhang (FIG. 7B). Additionally, a portion of the bottom strand of construction polynucleotide R that is complementary to the 5′ flanking region is removed thus producing a partially double stranded polynucleotide having a single stranded 5′ overhang (FIG. 7B). The construction polynucleotides are then contacted with the junction oligonucleotide under hybridization conditions thereby forming a bridge structure and aligning the bottom strands of construction polynucleotides L and R (FIG. 7C). The bridge structure is formed by the complementarity between the J regions of the junction oligonucleotide and the 3′ and 5′ flanking regions of the top strands of the construction polynucleotides. Additionally, the N region of the junction oligonucleotide base pairs with the 5′ and 3′ most terminal residues of the medial segments of the construction polynucleotides in a sequence independent manner (e.g., when N comprises universal bases) or in a sequence dependent manner involving a portion of the junction oligonucleotides having a sequence that can base pair (either Watson-Crick or Wobble base pairing) with the medial segments (e.g., when N comprises degenerate bases) (FIG. 7C). Upon exposure to ligation conditions, the bottom strands of construction polynucleotides L and R will be ligated together forming a polynucleotide comprising the sequences of the L and R construction polynucleotides flanked by 5′ flanking sequence of the L construction polynucleotide and the 3′ flanking sequence of the R construction polynucleotide (FIG. 7D). The polynucleotide may then be amplified using the FP_{L} and RP_{R} primers (FIG. 7E). Amplification with one primer from construction polynucleotide L and one primer from construction polynucleotide R permits confirmation that the correct product has been formed, and serves to effectively purify the desired product. Any products formed by nonspecific hybridization followed by ligation will not be amplifiable by the primer pair specific for the desired product and will be diluted out of the reaction. After amplification, the remaining flanking sequences may optionally be removed using the methods described below. Alternatively, the flanking sequences may be used in further assembly reactions. When using construction polynucleotides having very short single stranded overhangs, the adapter methods shown in FIG. 6 may be used in an analogous manner for formation of the bridge structure.

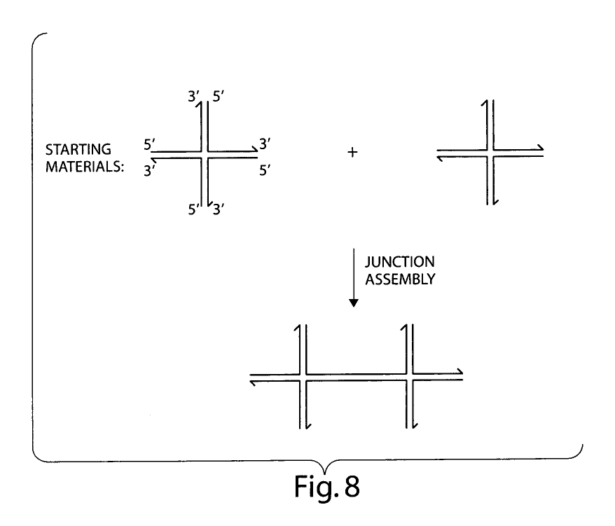

FIG. 8 illustrates one embodiment of the present invention wherein the junction assembly methods described herein may be used to join together two or more branched DNA structures. Based on the teachings herein, one of skill in the art will understand that the junction assembly methods described herein, particularly the methods shown in FIGS. 5 and 7, may be used for joining branched structures as well as linear DNAs. Branched DNA structures, and methods for making and using the same, are described, for example, in U.S. Pat. Nos. 6,255,469; 6,072,044; 5,468,851; 5,386,020; 5,278,051; U.S. Patent Publication No. 2003/02179790.

In certain embodiments, the junction assembly methods disclosed herein may have one or more of the following characteristics: the methods do not involve blunt end ligation, the methods are not dependent on restriction enzyme binding and/or cleavage sites, the methods do not involve restriction enzyme cleavage, the methods utilize 5′ and 3′ overhangs that are not complementary, the methods involve 5′ and 3′ overhangs that are not incorporated into the final product, and/or the methods involve junction oligonucleotide sequences that are not incorporated into the final product.

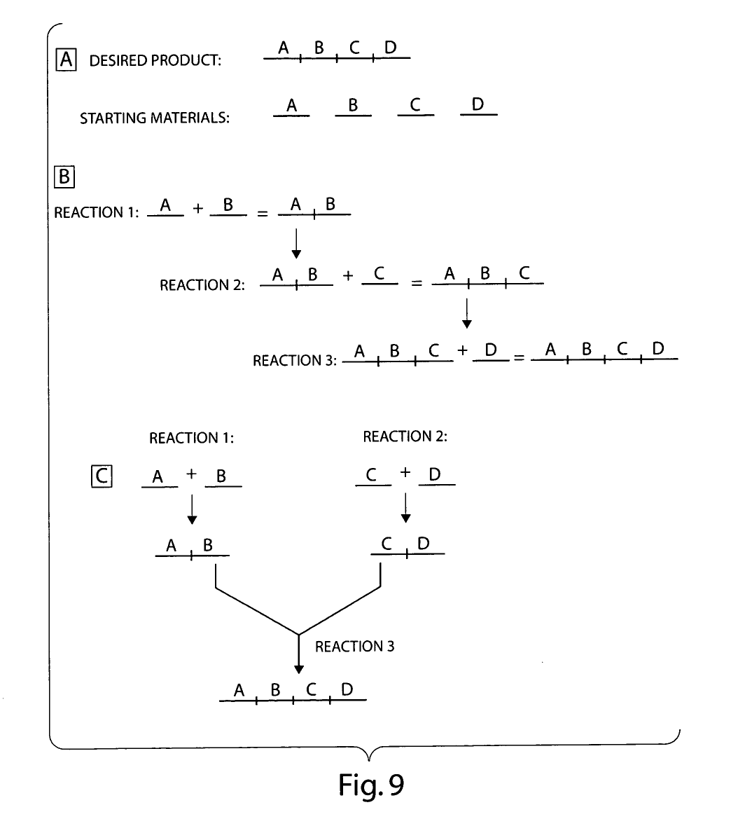

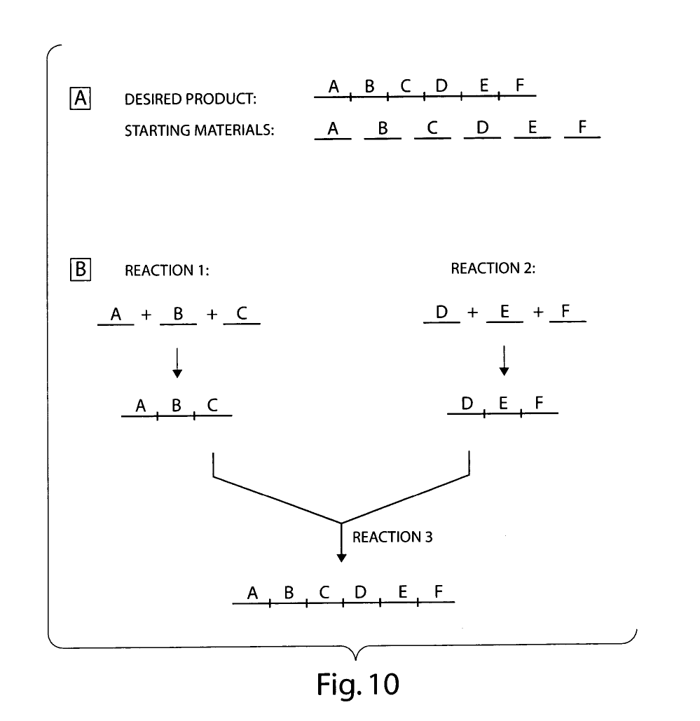

FIGS. 1-7 illustrate various methods for joining two construction polynucleotides in a desired order. In certain embodiments, a plurality of construction polynucleotides, e.g., 3, 4, 5, 6, 7, 8, 9, 10, 20, 50, 100, 1000, 10,000, or more, may be joined together to form one or more desired products. When joining more than two construction polynucleotides, the reactions may be carried out in parallel in a single reaction mixture. Alternatively, multiple joining reactions may be carried out using a hierarchical assembly involving multiple reactions that are mixed together in an ordered fashion (FIG. 9). Combinations thereof may also be conducted (FIG. 10). Hierarchical assembly methods may be desirable when connecting a large number of construction polynucleotides or when joining together three or more construction polynucleotides that have at least one junction sequence common to two or more of the construction polynucleotides that are desired to be joined.

Any resolvase protein may be used in association with the junction assembly methods illustrated in FIGS. 5 and 6. The resolvases are nucleolytic enzymes capable of catalyzing the resolution of branched DNA intermediates (e.g., DNA cruciforms or Holliday junctions). In general, these enzymes are active close to the site of DNA distortion (Bhattacharyya et al., J. Mol. Biol., 221, 1191, (1991)). A detailed analysis of resolvase protein sequences and structure modeling may be found in Aravind et al., Nucleic Acids Res. 28: 3417-3432 (2000). A wide variety of resolvases have been identified and characterized in bacteria, bacteriophages, yeast mitochondria, archaea, and metazoan viruses. In bacteria, the resolvase function is provided by at least four distinct protein families, including RuvC, YqgF, LE (λ exonuclease), and RusA (see e.g., Aravind et al., supra). Exemplary resolvase proteins include, for example, RuvC (conserved in the majority of bacteria; see e.g., Dunderdale et al., Nature 354: 506-510 (1991); Iwasake et al., EMBO J. 10: 4381-4389 (1991); Mizuuchi et al., Cell 29: 357-365 (1982)), E. coli RusA (Chan et al., J Biol Chem 272: 14873-14882 (1997)), E. Coli YqgF (Aravind et al., supra), lambdoid prophage RusA (see e.g., Sharples et al., EMBO J. 13: 6133-6142 (1994)), bacteriophage T4 endonuclease VII (see e.g., de Massy et al., J. Mol. Biol. 193: 359-376 (1987); Dickie et al., J. Biol. Chem. 262: 14826-14836 (1987)), resolvase from Pyrococcus furiosus (conserved in a wide variety of archaea genomes) (see e.g., Komori et al., Proc. Natal. Acad. Sci. USA 96: 8873-8878 (1999)), yeast mitochondrial resolvase Cce1 (see e.g., Oram et al., Nucleic Acids Res. 26: 594-601 (1998); White and Lilley, J. Mol. Biol. 266: 122-134 (1997); Kleff et al., EMBO J. 11: 669-704 (1992)), S. pombe mitochondrial YDC2 (see e.g., White and Lilley, Mol. Cell. Biol. 17: 6465-6471 (1997)), S. cerevisiae cruciform cleaving enzymes Endo X1, Endo X2, and Endo X3 (see e.g., West and Komer, Proc. Natl. Acad. Sci. USA, 82: 6445 (1985); West et al., J. Biol. Chem. 262: 12752 (1987)), topoisomerase IB from poxviruses (see e.g., Sekiguchi et al., Proc. Natl. Acad. Sci. USA 93: 785-789 (1996)), the RuvC homologs found in poxviruses and an iridovirus (see e.g., Garcia et al., Proc. Natl. Acad. Sci USA 97: 8926-8931 (2000)), and homologs, orthologs, paralogs of the foregoing (see e.g., Aravind et al., supra).

Resolvases for use in the practice of the present invention can be produced recombinantly and purified as previously described. Resolvases can be purified to a desired degree of purity by methods known in the art of protein purification including, for example, ammonium sulfate precipitation, size fractionation, affinity chromatography, HPLC, ion exchange chromatography, and heparin agarose affinity chromatography (see e.g., Thorpe and Smith, Proc. Nat. Acad. Sci. 95: 5505-5510 (1998)). Methods for purifying bacteriophage T7 endonuclease I (deMassy, B., et al. J. Mol. Biol. 193: 359 (1987) ), Endonuclease VII (Kosak et al., Eur. J. Biochem. 194, 779, (1990)), Endo X1 (West, S. C. and Komer, A. PNAS, 82, 6445 (1985); West, S. C. et al. J. Biol. Chem. 262: 12752 (1987)), Endo X2 (Symington, L. S. and Kolodner, R. PNAS 82: 7247 (1985)), Endo X3 (Jensch F. et al. EMBO J. 8, 4325 (1989)), and A22R protein from vaccinia virus (Garcia et al., Proc. Natl. Acad. Sci. USA 97: 8926-8931 (2000)) have been described.

Various ligation methods may be used in association with the junction assembly methods disclosed herein, including enzymatic ligation, chemical ligation, or ribozyme mediated ligation. Enzymatic ligation may be carried out using a protein ligase that forms phosphodiester bonds between the 3′-OH and the 5′-phosphate of adjacent nucleotides in DNA molecules, RNA molecules, or hybrids. Temperature sensitive ligases, include, but are not limited to, bacteriophage T4 ligase and E. coli ligase. Thermostable ligases include, but are not limited to, Taq ligase, Tfl ligase, Tth ligase, Tth HB8 ligase, Thermus species AK16D ligase and Pfu ligase. Methods of performing enzymatic ligation reactions are generally described in e.g., Sambrook, et al., Molecular Cloning: A Laboratory Manual, 2nd Ed., Cold Spring Harbor Laboratory, New York, 1989. Various ligases are commercially available, for example, from New England Biolabs (Beverly, Mass.). Chemical ligation agents include, without limitation, activating, condensing, and reducing agents, such as carbodiimide, cyanogen bromide (BrCN), N-cyanoimidazole, imidazole, 1-methylimidazole/carbodiimide/cystamine, dithiothreitol (DTT) and ultraviolet light. Autoligation, i.e., spontaneous ligation in the absence of a ligating agent, is also within the scope of the invention. Detailed protocols for chemical ligation methods and descriptions of appropriate reactive groups can be found in, for example, Xu et al., Nucleic Acid Res., 27:875-81 (1999); Shabarova, et al., Nucleic Acids Res. 19: 4247-4251 (1991); Gryaznov and Letsinger, Nucleic Acid Res. 21:1403-08 (1993); Gryaznov et al., Nucleic Acid Res. 22:2366-69 (1994); Kanaya and Yanagawa, Biochemistry 25:7423-30 (1986); Luebke and Dervan, Nucleic Acids Res. 20:3005-09 (1992); Sievers and von Kiedrowski, Nature 369:221-24 (1994); Liu and Taylor, Nucleic Acids Res. 26: 3300-04 (1999); Wang and Kool, Nucleic Acids Res. 22:2326-33 (1994); Purmal et al., Nucleic Acids Res. 20:3713-19 (1992); Ashley and Kushlan, Biochemistry 30:2927-33 (1991); Chu and Orgel, Nucleic Acids Res. 16:3671-91 (1988); Sokolova et al., FEBS Letters 232:153-55 (1988); Naylor and Gilham, Biochemistry 5:2722-28 (1966); and U.S. Pat. No. 5,476,930. Ribozyme mediated ligation is described in, for example, U.S. Pat. No. 5,498,531; U.S. Pat. No. 5,780,272; WO 95/07351; and WO 98/40519.

The junction assembly methods described herein may be carried out in vitro (e.g., using purified components, cell lysate, fractionated cell lysate, etc.) or in vivo (e.g., in a cell). When conducting the assembly methods in vivo, the DNA components to be joined (e.g., two or more construction polynucleotides and one or more junction oligonucleotides) may be introduced into a cell by a variety of transfection methods (see e.g., Mehier-Humbert and Guy, supra). The DNA components may be introduced into the cell as linear double stranded or single stranded segments or may be introduced into the cell as part of one or more larger construct, such as a plasmid, that are processed into the desired components after introduction into the cell (e.g., via a nuclease, etc.). In other embodiments, two or more components may be introduced into separate cells and mixed by conjugation of the cells to introduce the various DNA components into the same cell. Preferably the cell naturally contains all of the components needed for the assembly process, such as, for example, ligase, resolvase, polymerase, etc. Alternatively, the cell may be engineered so as to contain the proper complement of proteins needed to carry out the assembly process.

In certain embodiments, the junction assembly methods described herein may be carried out with a construction polynucleotide that is coupled to a solid support. For example, one construction polynucleotide may be coupled to a solid support. A junction oligonucleotide and second construction polynucleotide are then added to the support bound construction polynucleotide. A junction assembly method is conducted to join the two construction polynucleotides together in a desired order thereby forming a complex of two construction polynucleotides ligated together and coupled to the solid support. Successive rounds of addition and ligation of additional construction polynucleotides may be carried out until a desired product is formed. Each round of addition of construction polynucleotides may comprise incorporation of 1, 2, 3, 4, 5 or more construction polynucleotides at a time. Also, multiple separate construction polynucleotides may be couple to the same or different supports, and multiple larger sequences assembled simultaneously. In certain embodiments, it may be desirable to wash the support bound polynucleotides in between cycles of ligation to remove any non-conjugated intermediates (e.g., construction polynucleotides that did not get ligated to the support bound polynucleotide, junction oligonucleotides, etc.).

The polynucleotide constructs may be coupled to the solid support through a variety of means. For example, the polynucleotide constructs may be coupled to the solid support via a cleavable linker moiety (described in more detail below). The cleavable linker moiety may be added to the construction polynucleotide which is then contacted with a solid support to produce an immobilized construction polynucleotide. Alternatively, the polynucleotide construct may be synthesized directly on a solid support that has been functionalized with a cleavable linker moiety. In yet another embodiment, a construction polynucleotide may be coupled to the solid support via hybridization to an oligonucleotide that is attached to the solid support (e.g., an oligonucleotide covalently attached to the solid support, optionally, via a linker). The oligonucleotide attached to the solid support may be complementary to at least a portion of a polynucleotide construct. In an exemplary embodiment, the support bound oligonucleotide is complementary to at least a portion of a flanking region of a construction polynucleotide. The flanking region of the construction polynucleotide may be made at least partially single stranded such that the single stranded region of the construction polynucleotide may hybridize to the support bound oligonucleotide thereby coupling the construction polynucleotide to the solid support. Alternatively, the support bound oligonucleotide may be double stranded and may bind to the construction polynucleotide, for example, by hybridization of complementary overlapping sticky ends of the construction polynucleotide and the support bound oligonucleotide. In certain embodiments, the support bound oligonucleotide may be a universal oligonucleotide that is capable of hybridizing to the flanking sequences of a plurality of construction polynucleotides.

After assembly of two or more construction polynucleotides on a solid support, the product polynucleotide may be removed from the solid support by cleavage at the linker moiety, by melting to dissociate the complex between the construction polynucleotide and the support bound oligonucleotide, or by cleavage with a restriction endonuclease. The method used to remove the product polynucleotide will depend on how the construction polynucleotide was coupled to the solid support as described above.

When coupling a construction polynucleotide to a solid support based on hybridization to a support bound oligonucleotide, the support bound oligonucleotide preferably is not incorporated into the final product. This may be achieved by modifying the end of the oligonucleotide not bound to the support such that it is not a proper substrate for ligation, e.g., by removal of the hydroxyl group at the 3′ end, removal of the phosphate group at the 5′ end, addition of a phosphate group to the 3′ end, etc. In another embodiment, the end of the construction polynucleotide that hybridizes adjacent to the support bound oligonucleotide may be modified so that it is not a proper substrate for ligation. In yet another embodiment, the support bound oligonucleotide and construction polynucleotide may be designed such that there is a gap between the ends of the support bound oligonucleotide and the construction polynucleotide when the pair is hybridized together. The gap may be at least 1, 2, 3, 4, 5, or more nucleotides in length. In an exemplary embodiment, various combinations of modifications to the support bound oligonucleotide, modifications to the construction polynucleotide, and/or creation of a gap may be used to prevent ligation between the support bound oligonucleotide and the construction polynucleotide.

3. Removal of Flanking Regions

As is evident from the description above, one important enabler of the invention is the ability to remove flanking sequences from the medial segment, and in many instances to form single stranded 5′ and/or 3′ overhangs, with the flanking region being removed on one (or both) strands preferably precisely at the junction between the medial segment and the flanking sequence. If the Mullis method is used and pre-amplification of the construction polynucleotides is not needed, or primers are available to amplify the medial segment of the construction polynucleotide directly, then no flanking sequences are necessary. Where pre-amplification of the construction polynucleotides through the flanking sequences is to be conducted, the flanking sequences must necessarily be removed. Any method that can produce double stranded nucleic acids with blunt ends or single stranded overhangs may be used in connection with the assembly methods disclosed herein. For example, in one embodiment, nucleic acids may be synthesized de novo with both double stranded and single stranded regions. Alternatively, double stranded nucleic acids may be modified so as to produce single stranded overhangs at either the 5′ and/or 3′ ends using, for example, the methods described below.

In the embodiments of the invention comprising collections of multiple DNA parts for connection together to form any one of a variety of DNA encoded structures, it is preferred to maintain and provide the construction polynucleotides in double stranded form, the strands each comprising a medial segment (or its complement) and the 3′ and 5′ flanking regions (or their complements). These are designed specifically so as to permit removal of a 3′ or a 5′ flanking region in at least the sense strand, using standard, preferably orthogonal chemistries such as are exemplified below. This permits the bioengineer to select any group of construction polynucleotides from the kit and to devise a strategy to retrieve and assemble them in any order, ending with a construct comprising the medial segments of the selected construction polynucleotides joined end to end.

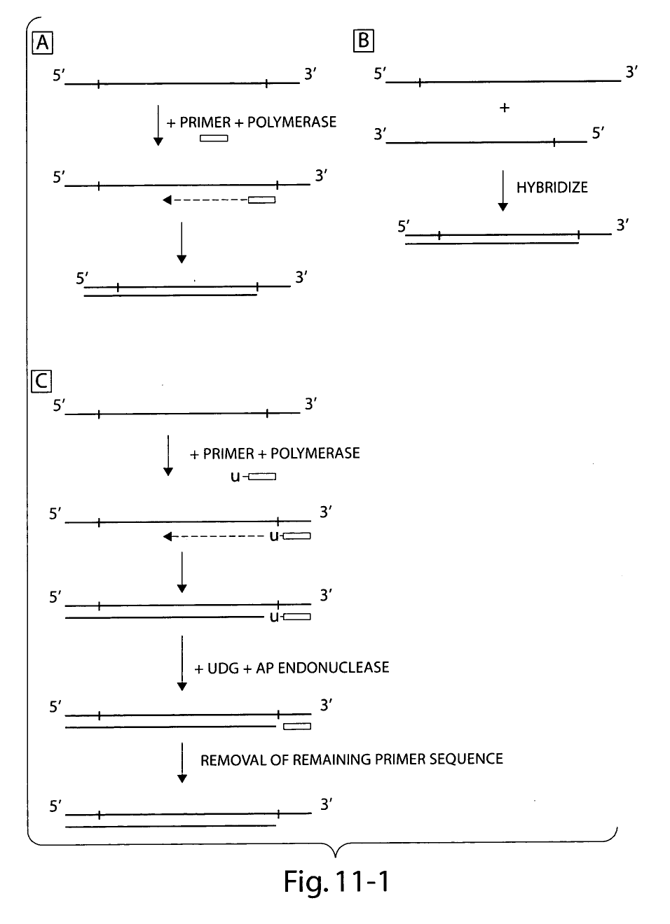

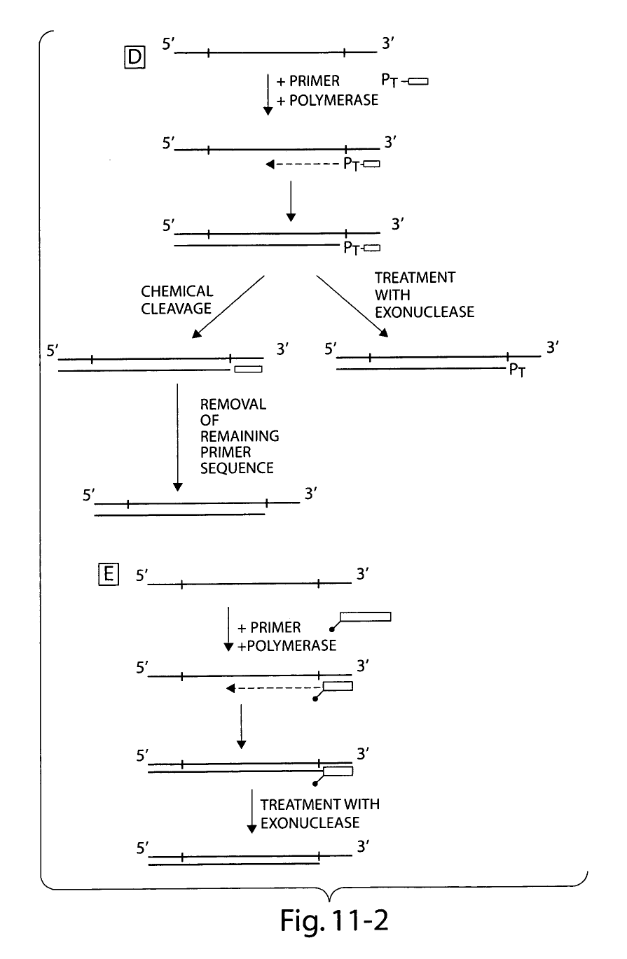

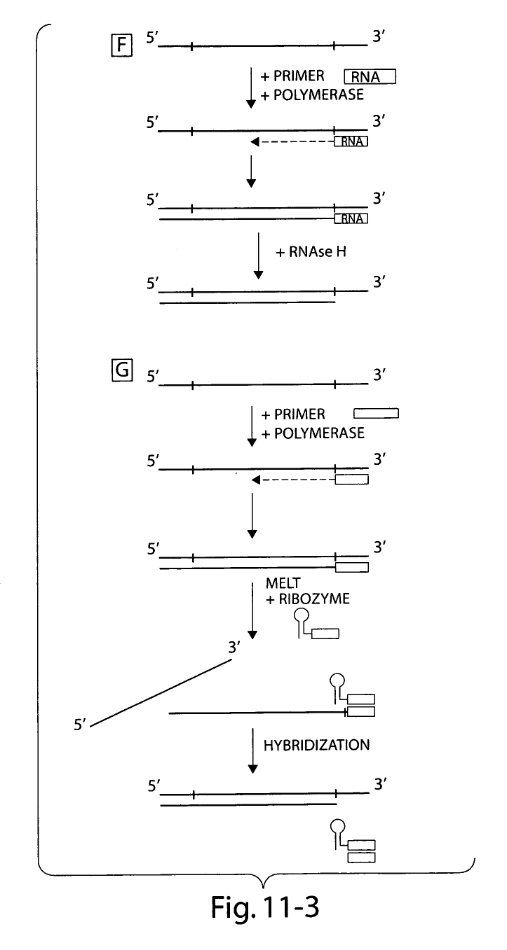

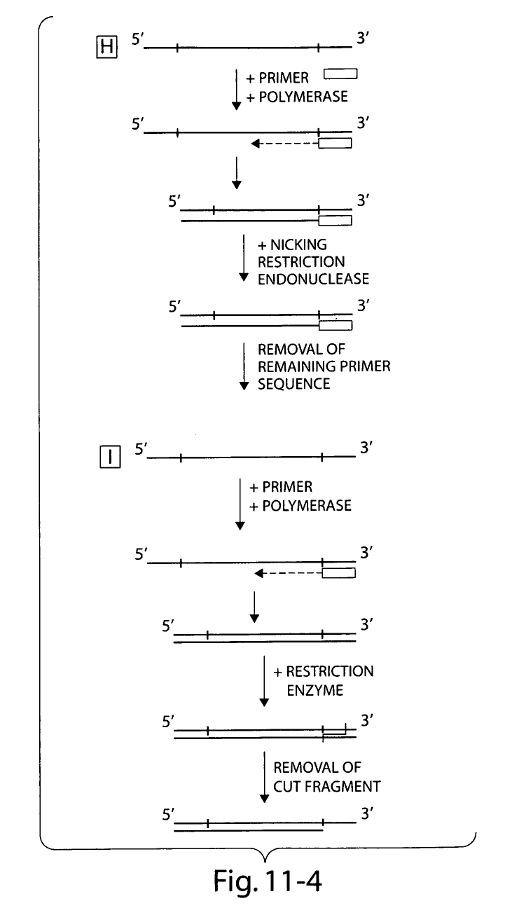

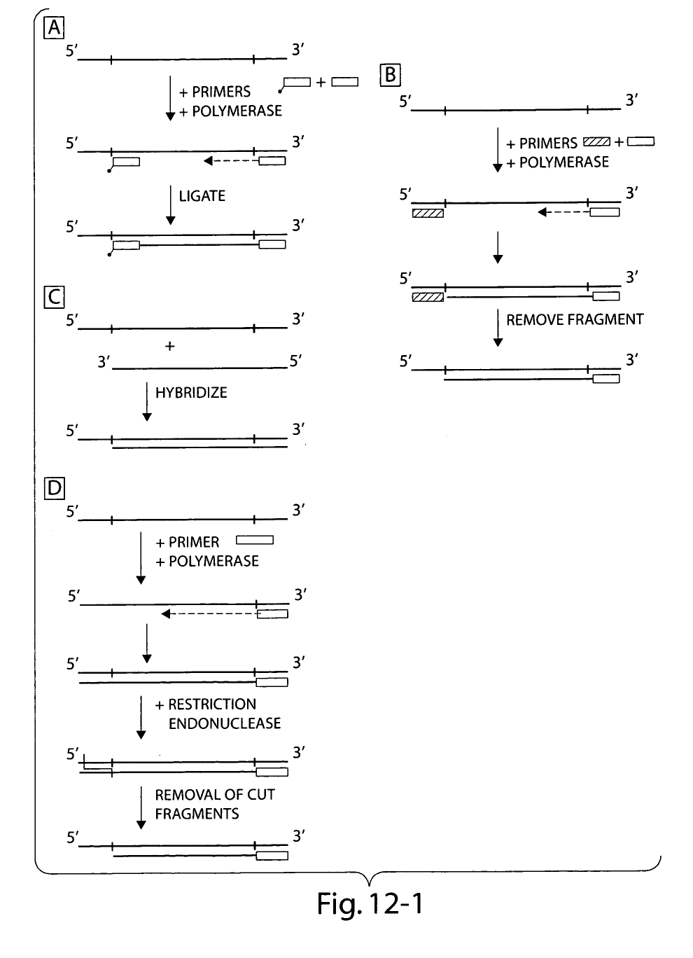

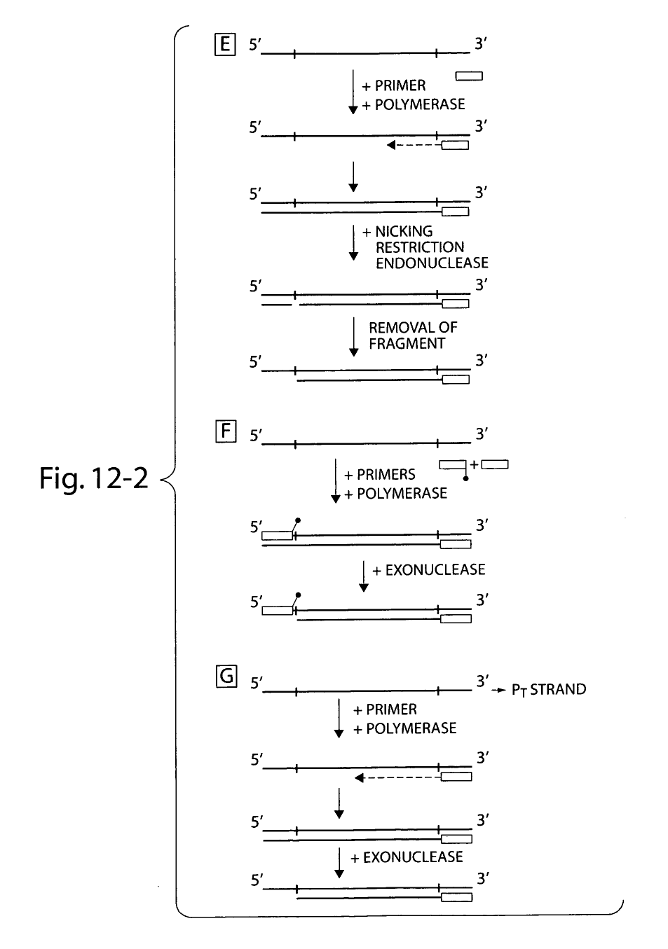

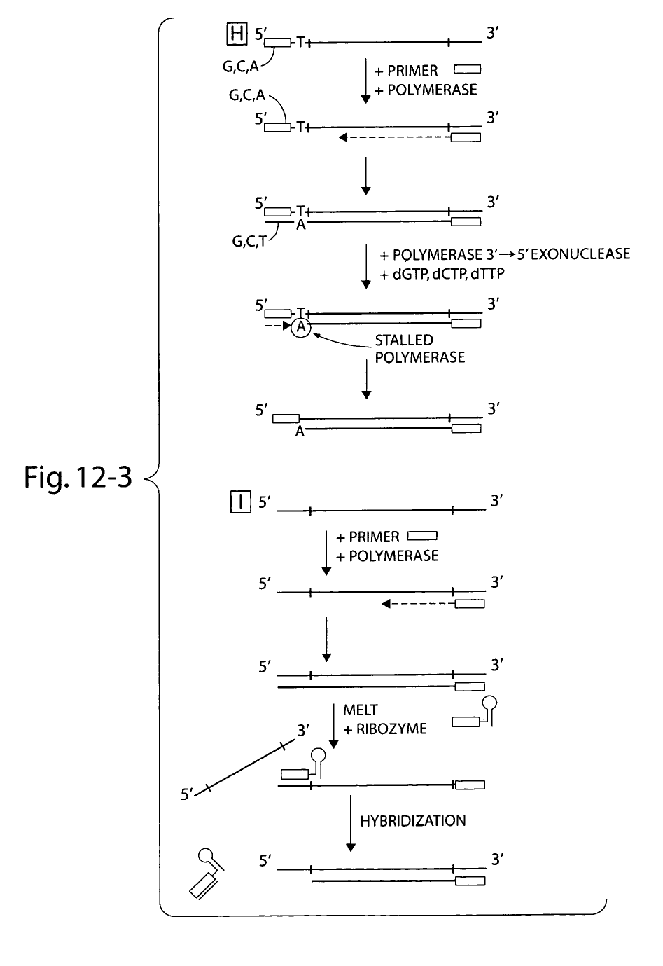

FIGS. 11A-I and 12A-I illustrate a variety of exemplary methods for producing double stranded nucleic acids with single stranded 3′ or 5′ overhangs, respectively. For illustration purposes only, the construction polynucleotides in FIGS. 11A-I and 12A-I are shown as single stranded polynucleotides (referred to as the top strand; when shown as a double stranded polynucleotide, the other strand will be referred to as the bottom or complementary strand). It should be understood that the top strand and bottom strands are designated as such merely for purposes of illustration. One of ordinary skill in the art would recognize that the nucleic acid strands are complementary and antiparallel and that the methods would apply equally to either strand. The 5′ and 3′ regions flanking the medial segment are demarcated by vertical lines and the directionality of only one strand is illustrated. In various embodiments, the construction polynucleotides may be single stranded or double stranded nucleic acids. When starting with single stranded construction polynucleotides, a partially double stranded nucleic acid with a 5′ and/or 3′ single stranded region may be produced as illustrated in the figures. Alternatively, the construction polynucleotides may be provided as modified or unmodified double stranded nucleic acids. For example, the construction polynucleotides may be provided as double stranded polynucleotides containing the modifications as illustrated, for example, in FIGS. 11C, 11D, 11E, 11F, 12E, and 12F, or as unmodified polynucleotides as illustrated in FIGS. 11G, 11H, 11I, 12C, 12D, 12G, and 12I. These polynucleotides may then be directly subjected to the methods illustrated in FIGS. 11 and 12 without the need to add primers or conduct chain extension. In other embodiments, unmodified double stranded construction polynucleotides may be used as the starting pool and subjected to the methods as illustrated in FIGS. 11 and 12 in order to introduce the desired modifications. In such embodiments, the double stranded polynucleotides may be separated to produce single strands prior to conducting primer hybridization and chain extension. In certain embodiments, the construction polynucleotides, whether single stranded or double stranded, may be amplified prior to production of the 5′ and/or 3′ single stranded overhangs. For example, the construction polynucleotides may be amplified using primers that hybridize to the 5′ and 3′ flanking regions, or the complements thereof.

In exemplary embodiments, methods for producing 5′ and/or 3′ single stranded overhangs are those methods that do not require sequence specific primers and those that are not dependent on the sequence of the medial segment. Exemplary methods for producing a 3′ overhang are illustrated in FIGS. 11C-11H. Exemplary methods for producing a 5′ overhang are illustrated in FIGS. 12B, and 12D-12I.

In certain embodiments, double stranded polynucleotides having both 5′ and 3′ single stranded overhangs may be produced. Such polynucleotides may be produced using various combinations of the methods illustrated in FIGS. 11A-I and 12A-I and by other methods. The 5′ and 3′ single stranded overhangs may be produced in a single reaction (i.e., the 5′ and 3′ single stranded overhangs are produced in the same reaction mixture) or may be produced using serial reactions (i.e., a 5′ or 3′ single stranded overhang is produced in a first reaction mixture and the second overhang is produced in a second reaction mixture). When conducting serial reactions, it may be desirable to purify an intermediate oligonucleotide product (i.e., a polynucleotide with one single stranded overhang) prior to further processing of the polynucleotide. When using a single reaction to produce both 5′ and 3′ single stranded overhangs, methods utilizing similar reaction conditions are preferred. One of skill in the art will be able to determine appropriate combinations of methods for producing a polynucleotide having two single stranded overhangs based on the teachings herein.

FIG. 11A-I illustrates a variety of exemplary methods for producing double stranded nucleic acids with a single stranded 3′ overhang. FIGS. 11A-B show two embodiments of methods for synthesis of a nucleic acid with a single stranded 3′ overhang. As illustrated in FIG. 11A, a chain extension reaction may be used to produce a partially double stranded nucleic acid. The primer used in the chain extension reaction may be designed such that the 5′ end of the primer hybridizes to the 3′ end of the medial segment of the construction polynucleotide. Following chain extension, the resulting nucleic acid will comprise a double stranded region spanning the medial segment and the 5′ flanking region of the construction polynucleotide and a single stranded region spanning the 3′ flanking region of the construction polynucleotide. FIG. 11B shows an alternative method for synthesizing a polynucleotide having a single stranded 3′ overhang. As shown in FIG. 11B, both strands of the polynucleotide are separately synthesized, e.g., chemically synthesized, and then mixed together under hybridization conditions. As illustrated, the top strand is synthesized with the 5′ flanking region, 3′ flanking region and the medial segment. The bottom strand is synthesized to be complementary to regions spanning the 5′ flanking region and the medial segments only. After hybridization of the two complementary strands, a double stranded polynucleotide having a single stranded 3′ overhang is formed.

FIG. 11C shows another method for producing a double stranded polynucleotide having a single stranded 3′ overhang that involves incorporation of a uracil residue. As shown in FIG. 11C, a primer complementary to the 3′ flanking region of the construction polynucleotide may be used in a chain extension reaction. The primer comprises at least one uracil residue at the junction between the 3′ flanking region and the medial segment. After chain extension the polynucleotide is treated with uracil DNA glycosylase and an AP endonuclease to remove the uracil residue and produce a single stranded nick between the 3′ flanking region and the medial segment on the complementary strand. The fragment complementary to the 3′ flanking region may then be removed using size separation (e.g., column chromatography, gel electrophoresis, etc.). In certain embodiments, the primer complementary to the 3′ flanking region may comprise 1, 2, 3, 4, 5, or more uracil residues wherein at least one of the uracil residues is located at the junction between the 3′ flanking region and the medial segment of the construction polynucleotide. In an exemplary embodiment, the uracil residue may be excised from the double stranded DNA using the USER™ (Uracil-Specific Excision Reagent) enzyme. Uracil-DNA glycosylase, USER™ enzyme, and various AP endonucleases (e.g., Endonuclease VIII) are commercially available, for example, from New England Biolabs (Beverly, Mass.). In various other embodiments, other combinations of bases and DNA glycosylases may be used as means to produce a single stranded 3′ overhang, including for example, Hmu-DNA glycosylase (recognizes hydroxymethyl uracil), 5-mC-DNA glycosylase (recognizes 5-methylcytosine), Hx-DNA glycosylase (recognizes hypoxanthine), 3-mA-DNA-glycosylase I (recognizes 3-methyladenine), 3-mA-DNA-glycosylase II (recognizes 3-methyladenine, 7-methylguanine and 3-methylguanine), FaPy-DNA glycosylase (recognizes formamidopyrimidines and 8 hydroxyguanine), and 5,6-HT-DNA-glycosylase (recognizes 5,6 hydrated thymines).

FIG. 11D illustrates yet another method for producing a double stranded polynucleotide having a single stranded 3′ overhang. As shown in FIG. 11D, a primer complementary to the 3′ flanking region of the construction polynucleotide may be used in a chain extension reaction. The primer comprises at least one phosphorothioate internucleoside linkage between the last two nucleotides complementary to the 3′ flanking region at the junction with the medial segment. After chain extension, the portion complementary to the 3′ flanking region may be removed by two alternative methods. As shown on the left in FIG. 11D, the phosphorothioate intemucleoside linkage may be cleaved using an alkylating reagent (e.g., 2-iodoethanol, 2,3-epoxy-1-propanol, etc.) to produce a single stranded nick (see e.g., Gish and Eckstein, Science 240: 1520-1522 (1988); Nakamaye et al., Nucl. Acids Res. 16: 9947-9959 (1988)). The fragment complementary to the 3′ flanking region may then be removed using size separation (e.g., column chromatography, gel electrophoresis, etc.). Alternatively, as shown on the right in FIG. 11D, the region complementary to the 3′ flanking region may be removed using a 5′ to 3′ exonuclease such as, for example, lambda or T7exonuclease. The phosphorothioate internucleoside linkage is resistant to exonuclease cleavage and will prevent the exonuclease from digesting the complementary strand beyond the location of the phosphorothioate linkage (see e.g., Labeit, et al., DNA 5: 173-177 (1986)). In this embodiment, the 5′ end of the top strand of the construction polynucleotide may be modified to prevent unwanted exonuclease digestion at the 5′ end. Modifications that prevent exonuclease digestion include, for example, a 5′ chemical cap or one or more phosphorothioate linkages incorporated at the 5′ end of the polynucleotide. Such 5′ modifications of the top strand may be incorporated during synthesis of the construction polynucleotide, may be incorporated using a modified primer followed by chain extension, or may be introduced by chemical or enzymatic modification of the polynucleotide after synthesis. In certain embodiments, the primer complementary to the 3′ flanking region may comprise 1, 2, 3, 4, 5, or more phosphorothioate linkages. Various exonucleases are commercially available, for example, from New England Biolabs (Beverly, Mass.).

In other embodiments, other types of modified intemucleoside linkages may be used to produce a double stranded polynucleotide having a single stranded 3′ overhang as illustrated in FIG. 11D for a phosphorothioate linkage. For example, a variety of intemucleoside linkages that may cleaved by chemical, thermal, or light based methods may be used. Exemplary chemically cleavable internucleoside linkages for use in the methods described herein include, for example, B-cyano ether, 5′-deoxy-5′-aminocarbamate, 3′ deoxy-3′-aminocarbamate, urea, 2′ cyano-3′, 5′-phosphodiester, 3′-(S)-phosphorothioate, 5′-(S)-phosphorothioate, 3′-(N)-phosphoramidate, 5′-(N)-phosphoramidate, α-amino amide, vicinal diol, ribonucleoside insertion, 2′-amino-3′,5′-phosphodiester, allylic sulfoxide, ester, silyl ether, dithioacetal, 5′-thio-furmal, α-hydroxy-methyl-phosphonic bisamide, acetal, 3′-thio-furmal, methylphosphonate and phosphotriester. Internucleoside silyl groups such as trialkylsilyl ether and dialkoxysilane are cleaved by treatment with fluoride ion. Base-cleavable sites include P-cyano ether, 5′-deoxy-5′-aminocarbamate, 3′-deoxy-3′-aminocarbamate, urea, 2′-cyano-3′, 5′-phosphodiester, 2′-amino-3′,5′-phosphodiester, ester and ribose. Thio-containing internucleoside bonds such as 3′-(S)-phosphorothioate and 5′-(S)-phosphorothioate are cleaved by treatment with silver nitrate or mercuric chloride. Acid cleavable sites include 3′-(N)-phosphoramidate, 5′-(N)-phosphoramidate, dithioacetal, acetal and phosphonic bisamide. An α-aminoamide internucleoside bond is cleavable by treatment with isothiocyanate, and titanium may be used to cleave a 2′-amino-3′,5′-phosphodiester-O-ortho-benzyl internucleoside bond. Vicinal diol linkages are cleavable by treatment with periodate. Thermally cleavable groups include allylic sulfoxide and cyclohexene while photo-labile linkages include nitrobenzylether and thymidine dimer. Methods synthesizing and cleaving nucleic acids containing chemically cleavable, thermally cleavable, and photo-labile groups are described for example, in U.S. Pat. No. 5,700,642.

FIG. 11E shows another method for producing a double stranded polynucleotide having a single stranded 3′ overhang that involves incorporation of a bulky group. As shown in FIG. 11E, a primer complementary to the 3′ flanking region of the construction polynucleotide may be used in a chain extension reaction. The primer comprises at least one bulky group at the junction between the 3′ flanking region and the medial segment. After chain extension, the region complementary to the 3′ flanking region may be removed using a 5′ to 3′ exonuclease such as, for example, lambda or T7 exonuclease. The bulky group blocks the progression of the exonuclease and prevents degradation of the complementary strand beyond the location of the bulky group. As described above, the 5′ end of the top strand may be modified to prevent unwanted exonuclease cleavage of the top strand. The bulky group is a modification that permits chain extension by polymerase but blocks the action of an exonuclease. In an exemplary embodiment, the primer comprises a binding site for a larger bulky group that may be added after chain extension with the polymerase. For example, the primer may contain a biotin molecule which can be further modified by addition of avidin or an antibody after chain extension to increase the size of the bulky group. The biotin or bulky group may be attached to the polynucleotide by a cleavable linker (e.g., chemical or photolabile linker) so that the bulky group can be removed after treatment with the exonuclease if desired. The bulky group may be added to the bottom strand during chemical synthesis of the construction polynucleotide (e.g., when the bottom strand is synthesized) or may be introduced through PCR using a primer containing the bulky group as illustrated in FIG. 11E.

FIG. 11F shows another method for producing a double stranded polynucleotide having a single stranded 3′ overhang that utilizes an RNA primer. As shown in FIG. 11F, an RNA primer complementary to the 3′ flanking region of the construction polynucleotide may be used in a chain extension reaction. After chain extension, the region complementary to the 3′ flanking region may be removed using an RNase (e.g., RNase H) to produce a 3′ overhang.

FIG. 11G shows another method for producing a double stranded polynucleotide having a single stranded 3′ overhang using a ribozyme. As shown in FIG. 11G, a strand complementary to the top strand of the construction polynucleotide may be synthesized by chain extension. The strands are then separated and contacted with a catalytic ribozyme that binds to the bottom strand in the region complementary to the 3′ flanking region and cleaves the polynucleotide at the junction between the 3′ flanking region and the medial segment. The cleaved fragment may then be removed by size separation (e.g., column chromatography, gel electrophoresis, etc.) and the top and bottom strands incubated under hybridization conditions to form a double stranded polynucleotide having a single stranded 3′ overhang.

FIG. 11H shows another method for producing a double stranded polynucleotide having a single stranded 3′ overhang using a nicking restriction endonuclease. The 3′ flanking region is designed to contain a recognition site for a nicking restriction endonuclease, preferably one that cuts offset from the recognition site. The cleavage site is positioned such that the enzyme will create a nick at the junction between the 3′ flanking region and the medial segment on the complementary strand. After cleavage with the nicking restriction enzyme, the fragment complementary to the 3′ flanking region may be removed using size separation (e.g., column chromatography, gel electrophoresis, etc.). In an exemplary embodiment, the recognition sequence for the nicking restriction enzyme is located entirely in the 3′ flanking region and does not depend on the sequence of the medial segment. Exemplary nicking restriction endonucleases include, for example, N.Alw I or N.BstNB I. Various nicking restriction enzymes are commercially available, for example, from New England Biolabs (Beverly, Mass.).

FIG. 11I shows another method for producing a double stranded polynucleotide having a single stranded 3′ overhang using a restriction endonuclease. The 3′ flanking region is designed to contain a recognition site for a restriction endonuclease, preferably one that produces at least a 4 or 5 base overhang. The cleavage site is positioned so that the restriction enzyme will cleave the double stranded polynucleotide on the bottom strand at the junction between the 3′ flanking region and the medial segment and on the top strand at a position located within the 3′ flanking region. After cleavage with the restriction enzyme, the small double stranded fragment obtained from the 3′ flanking region may be removed using size separation (e.g., column chromatography, gel electrophoresis, etc.). A wide variety of restriction endonucleases having specific binding and/or cleavage sites are commercially available, for example, from New England Biolabs (Beverly, Mass.). In an exemplary embodiment, the recognition sequence for the restriction enzyme is located entirely in the 3′ flanking region and does not depend on the sequence of the medial segment. Exemplary restriction endonucleases for producing a 3′ overhang include Type IIS restriction endonucleases or restriction endonucleases that cleave at sites surrounding their recognition site so that the cleavage reaction is not dependent on the sequence of the medial segment. An exemplary restriction endonuclease includes, for example, Hpy99 I which produces a 5 base overhang (recognition site: 5′ˆCGWCGˆ3′, wherein W=A or T and ˆ represents a site of cleavage). In certain embodiments, it may be desirable to extend a 3′ overhang after cleavage with the restriction endonuclease. A terminal extension on the 3′ overhang may be added using a terminal transferase enzyme (New England Biolabs, Beverly, Mass.) in the presence of dNTPs. In an exemplary embodiment, the terminal transferase may be used to extend a short 3′ overhang (e.g., less than 10 nucleotides) to produce an overhang suitable for conducting the joining reactions illustrated in any one of FIGS. 1-7 (e.g., an overhang of at least about 5, nucleotides, 10 nucleotides, 15 nucleotides, 20 nucleotides, 25 nucleotides, or longer).

FIG. 12A-I illustrates a variety of exemplary methods for producing double stranded nucleic acids with a single stranded 5′ overhang. FIGS. 12A-C show three embodiments of methods for synthesis of a nucleic acid with a single stranded 5′ overhang. As illustrated in FIG. 12A, chain extension may be carried out in the presence of two primers that hybridize to the top strand. A first primer hybridizes to the 3′ flanking region and serves as a site for initiation of chain extension. A second primer hybridizes to the medial segment at the junction with the 5′ flanking region. The second primer is modified so that it will not allow initiation of chain extension from the 3′ end. Polymerase will extend the first primer until it encounters the second primer and then will terminate. The extended bottom strand may be ligated to the second primer to form the double stranded polynucleotide having a 5′ overhang. The 3′ end of second primer may be treated to permit ligation to another polynucleotide.

FIG. 12B illustrates an alternative method for synthesizing a double stranded polynucleotide having a single stranded 5′ overhang using two primers. A first primer hybridizes to the 3′ flanking region and serves as a site for initiation of chain extension. A second primer hybridizes to the 5′ flanking region and serves as a bumper to prevent chain extension into the 5′ flanking region. After chain extension, the second primer may be removed by size separation (e.g., column chromatography, gel electrophoresis, etc.). The methods illustrated in FIGS. 12A and 12B utilize a polymerase that does not have strand displacement activity such as, for example, T4 DNA polymerase, DNA polymerase I, T7 DNA polymerase, or Taq DNA polymerase (New England Biolabs, Beverly, Mass.).

FIG. 12C shows another method for synthesizing a double stranded polynucleotide having a single stranded 5′ overhang. This method is analogous to the method illustrated in FIG. 12B for production of a 3′ overhang. As shown in FIG. 12C, both strands of the polynucleotide may be separately synthesized and then mixed together under hybridization conditions. The top strand is synthesized with the 5′ flanking region, 3′ flanking region, and the medial segment. The bottom strand is synthesized to be complementary to the regions spanning the 3′ flanking region and the medial segments only. After hybridization of the two complementary strands, a double stranded polynucleotide having a single stranded 5′ overhang is formed.

FIG. 12D shows another method for producing a double stranded polynucleotide having a single stranded 5′ overhang using a restriction endonuclease. This method is analogous to the method illustrated in FIG. 11I for production of a 3′ overhang. The 5′ flanking region is designed to contain a recognition site for a restriction endonuclease, preferably one that produces at least a 4 or 5 base overhang. The cleavage site is positioned so that the restriction enzyme will cleave the double stranded polynucleotide on the bottom strand at the junction between the 5′ flanking region and the medial segment and on the top strand at a position located within the 5′ flanking region. After cleavage with the restriction enzyme, the small double stranded fragment obtained from the 5′ flanking region may be removed using size separation (e.g., column chromatography, gel electrophoresis, etc.). A wide variety of restriction endonucleases having specific binding and/or cleavage sites are commercially available, for example, from New England Biolabs (Beverly, Mass.). In an exemplary embodiment, the recognition sequence for the restriction enzyme is located entirely in the 5′ flanking region and does not depend on the sequence of the medial segment. Exemplary restriction endonucleases for producing a 5′ overhang include Type IIS restriction endonucleases or restriction endonucleases that cleave at sites surrounding their recognition site so that the cleavage reaction is not dependent on the sequence of the medial segment. Exemplary Type IIS restriction endonucleases for producing a 5′ overhang include, for example, BsmA I, BspM I, SfaN I, Hga I, Bbv I, Fok I, BsmF I, Eco31I, Esp3 I, and Aar I. Exemplary restriction endonucleases that produce 5 base overhangs and have cleavage sites surrounding their recognition site include, for example, Bssk I, PspG I, StyD4 I, Tsp45 I, BstSC I, EcoR II, Mae III, NmuC I, and Psp6 I.

FIG. 12E shows another method for producing a double stranded polynucleotide having a single stranded 5′ overhang using a nicking restriction endonuclease. This method is analogous to the method illustrated in FIG. 11H for production of a 3′ overhang. The 5′ flanking region is designed to contain a recognition site for a nicking restriction endonuclease, preferably one that cuts offset from the recognition site. The cleavage site is positioned such that the enzyme will create a nick at the junction between the 5′ flanking region and the medial segment on the complementary strand. After cleavage with the nicking restriction enzyme, the fragment complementary to the 5′ flanking region may be removed using size separation (e.g., column chromatography, gel electrophoresis, etc.). In an exemplary embodiment, the recognition sequence for the nicking restriction enzyme is located entirely in the 5′ flanking region and does not depend on the sequence of the medial segment. An exemplary nicking restriction enzyme is Nb.Bsm I. Various nicking restriction enzymes are commercially available, for example, from New England Biolabs (Beverly, Mass.).

FIG. 12F shows another method for producing a double stranded polynucleotide having a single stranded 5′ overhang using a bulky group. This method is similar to the method illustrated in FIG. 11E for production of a 3′ overhang. As shown in FIG. 12F, the top strand of the construction polynucleotide is constructed to contain at least one bulky group at the junction between the 5′ flanking region and the medial segment. The bulky group may be added during chemical synthesis of the construction polynucleotide or may be introduced through PCR using a primer containing the bulky group at the desired location. The region complementary to the 5′ flanking region may be removed using a 3′ to 5′ exonuclease such as, for example, exonuclease im or BAL-31 nuclease. The bulky group on the top strand blocks the progression of the exonuclease on the bottom strand and prevents the degradation of the bottom strand beyond the location of the bulky group. As described above, the 3′ end of the top strand may be modified to prevent unwanted exonuclease cleavage of the top strand. The bulky group is a modification that permits chain extension by polymerase but blocks the action of an exonuclease. In an exemplary embodiment, the primer comprises a binding site for a larger bulky group that may be added after chain extension with the polymerase. For example, the primer may contain a biotin molecule which can be further modified by addition of avidin or an antibody after chain extension to increase the size of the bulky group. The biotin may be attached to the polynucleotide by a cleavable linker (e.g., chemical or photolabile linker) so that the bulky group can be removed after treatment with the exonuclease if desired.

FIG. 12G illustrates yet another method for producing a double stranded polynucleotide having a single stranded 5′ overhang using nuclease resistant internucleoside linkages. As shown in FIG. 12G, the top strand may be synthesized (e.g., either chemical or enzymatic synthesis) with one or more modified internucleoside linkages as described above for FIG. 11D (e.g., phosphorothioate, etc.). As an alternative to the modified internucleoside linkages, the 3′ end of the top strand may be modified (e.g., with a capping group) to prevent 3′ to 5′ exonuclease activity. The 3′ flanking region of the bottom strand may be removed using a 3′ to 5′ exonuclease that will selectively act on the bottom strand due to the modifications making the top strand exonuclease resistant at the 3′ end. Exemplary 3′ to 5′ exonucleases include, for example, exonuclease III or Bal-31 nuclease (New England Biolabs, Beverly, Mass.). The exonuclease reaction may be stopped at the junction of the 5′ flanking region with the medial region based on time of the reaction or by incorporation of an exonuclease resistant modification at the junction (e.g., a phosphorothioate intemucleoside linkage) of the 5′ flanking region with the medial region on the bottom strand.

FIG. 12H shows another method for producing a double stranded polynucleotide having a single stranded 5′ overhang using the 3′ to 5′ proofreading activity of polymerase. The 5′ flanking region of the construction polynucleotide is designed to have a sequence comprising only 3 of 4 possible dNTPs (e.g., dGTP, dCTP, dTTP; bottom strand sequence) and the first residue in the medial segment is the fourth dNTP not found in the flanking region (e.g., dATP; bottom strand sequence). The double stranded construction polynucleotide is incubated with a polymerase having 3′ to 5′ exonuclease activity in the presence of only the fourth DNTP found at the first residue in the medial segment (here DATP). The polymerase will chew back the 3′ end of the bottom strand until it encounters the first residue in the strand that corresponds to a dNTP present in the reaction mixture, e.g., the adenine residue located at the junction between the 5′ flanking region and the medial segment. When the polymerase encounters this first adenine residue, it will stall and can be removed from the polynucleotide leaving a 5′ overhang. The 3′ end of the top strand can be modified as described herein to prevent exonuclease activity of the top strand or by designing the 3′ terminal end of its flanking region to be an A. Exemplary polymerases having 3′ to 5′ exonuclease activity include, for example, phi29 DNA polymerase, T4 DNA polymerase, DNA polymerase I, DNA polymerase I Kienow fragment, T7 DNA polymerase, VentR DNA polymerase, Deep VentR DNA polymerase, and 9ONm DNA polymerase (New England Biolabs, Beverly, Mass.).

FIG. 12I shows another method for producing a double stranded polynucleotide having a single stranded 5′ overhang using a ribozyme. This method is analogous to the method illustrated in FIG. 11G for production of a 3′ overhang. As shown in FIG. 12I, a strand complementary to the top strand of the construction polynucleotide may be synthesized by chain extension. The strands are then separated and contacted with a catalytic ribozyme that binds to the bottom strand in the region complementary to the 5′ flanking region and cleaves the polynucleotide at the junction between the 5′ flanking region and the medial segment. The cleaved fragment may then be removed by size separation (e.g., column chromatography, gel electrophoresis, etc.) and the top and bottom strands incubated under hybridization conditions to form a double stranded polynucleotide having a single stranded 5′ overhang.

In certain embodiments, the assembly methods described herein (e.g., FIGS. 1, 2 and 4) utilize construction polynucleotides wherein both strands of the 5′ and/or 3′ flanking regions have been removed (e.g., a blunt end). A 5′ and/or 3′ flanking region may be removed and a blunt end produced using, for example, a restriction endonuclease that produces a blunt end. A wide variety of restriction endonucleases having specific binding and/or cleavage sites. are commercially available, for example, from New England Biolabs (Beverly, Mass.). In an exemplary embodiment, the recognition sequence for the restriction enzyme is located entirely in the 5′ or 3′ flanking region and does not depend on the sequence of the medial segment. Exemplary restriction endonucleases for producing a blunt end include Type IIS restriction endonucleases or restriction endonucleases that cleave at sites surrounding their recognition site so that the cleavage reaction is not dependent on the sequence of the medial segment. Exemplary restriction endonucleases for producing a blunt end include, for example, Mly I and Sch I. Alternatively, double stranded blunt ends may be formed using any of the methods illustrated in FIGS. 11A-I and 12A-I followed by treatment with an exonuclease to remove the overhang (e.g., RecJ_{f} for removal of 5′ overhangs and exonuclease I or exonuclease T for removal of 3′ overhangs). These methods may also be used to remove the flanking regions after assembly of two or more construction polynucleotides if desired. For example, the 5′ flanking region of a construction polynucleotide joined on the left and the 3′ flanking region of a construction polynucleotide joined on the right (e.g., the FP_{L} and RP_{R} regions illustrated in FIG. 2). Additionally, these methods may be used to remove the flanking regions of a junction oligonucleotide.

4. Compositions and Kits

In other aspects, the invention provides compositions of construction polynucleotides, junction oligonucleotides, and/or primer pairs for producing one or more polynucleotide constructs. In an exemplary embodiment, the invention provides a mixture, or a plurality of mixtures, of construction polynucleotides and/or junction oligonucleotides that are selectively retrievable out of the mixture.

In one embodiment, the invention provides a set of construction polynucleotides that are adapted for connection together using the junction assembly methods described herein. The construction polynucleotides comprise a medial sequence and 3′ and 5′ flanking sequences, wherein the construction polynucleotides are designed to permit formation of single stranded ends corresponding to at least a portion of the 3′ and/or 5′ flanking regions. The medial sequences of the construction polynucleotides may be connected together in any order using the junction assembly methods described herein, and the connection process is not dependent on the sequence of the medial sequences. This permits joining together of any combination of connection polynucleotides without the need to rely on the natural placement of restriction enzyme sites, the ability to introduce restriction enzyme sites, and without worrying about the frame of the sequences to be joined. In one embodiment, the flanking regions of the construction polynucleotides may comprise binding sites for one or more primer pairs. In certain embodiments, the flanking regions of the construction polynucleotides comprise nested binding sites for at least two, three, or more primer pairs. The nested primer binding sites may be used to amplify, and thereby isolate, a given construction polynucleotide from a mixture of construction polynucleotides. In an exemplary embodiment, the 3′ and 5′ flanking regions may be removed using, for example, any of the methods described herein and as illustrated in FIGS. 11 and 12, or other methods. In certain embodiments, one of the primers in a primer pair used to amplify the construction polynucleotides may be functionalized with a group that facilitates isolation of one strand of the junction oligonucleotides, e.g., such as biotin (see FIG. 4).

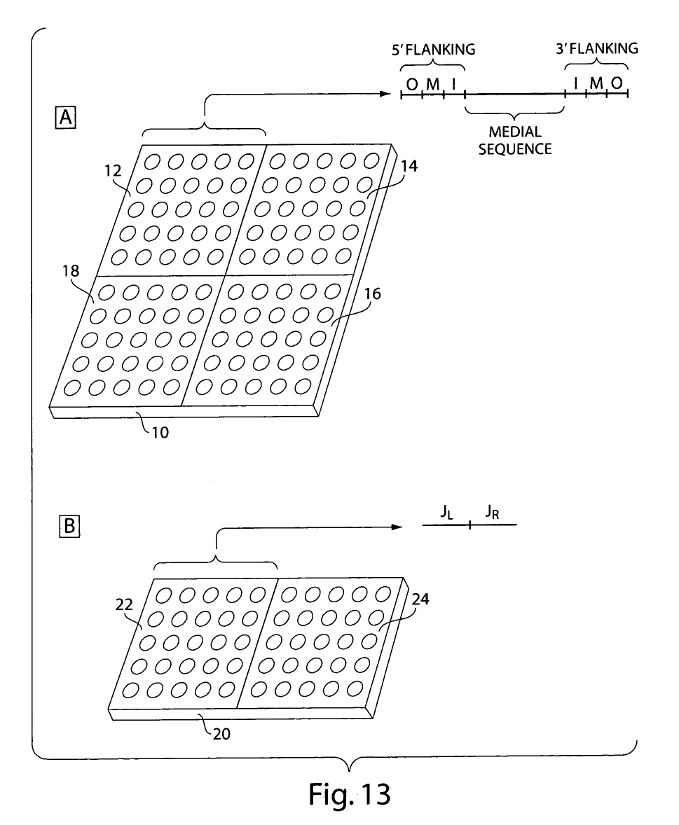

An exemplary embodiment of a set of construction polynucleotides is illustrated in FIG. 13A. FIG. 13A shows a 384 well plate containing 96³ construction polynucleotide sequences (or 884,736 sequences) located in quadrant 12 and three sets of 96 pairs of primers located in quadrants 14, 16, and 18 (e.g., one primer pair per well, or 96×3=288 primer pairs). Each well in quadrant 12 contains 96² (or 9,216) construction polynucleotides. The construction polynucleotides each comprise, e.g., 3 sets of nested primer biding sites, referred to as outer (O), middle (M), and inner (I) primer sets. Each construction polynucleotide in a given well comprises a different combination of O, M and I primer sets which permits any given construction polynucleotide in a particular well to be amplified, and thus isolated, from the mixture of 9,216 polynucleotides. For example, the optional outer set of primers may be common to a single well (or to all of the wells) and can be used to amplify the entire mixture of construction polynucleotides, therby permitting maintenance of the inventory of the construction polynucleotides. Amplification with a set of middle primers will amplify 1/96 (or 96 of the 9,216) construction polynucleotides in a given well. The amplification with the set of middle primers produces a pool of 96 construction polynucleotides comprising medial sequences and 3′ and 5′ flanking regions each having two sets of nested primer binding sites (e.g., binding sites for middle and inner primers). A subsequent amplification of this pool using a set of inner primers will amplify a single construction polynucleotide (e.g., 1/96 of the pool) comprising a medial sequence and 5′ and 3′ flanking regions comprising binding sites for an inner primer pair. Therefore, a single 384 plate may be used to store, renew, and selectively isolate any sequence from the mixture using 2-3 quick and simple rounds of amplification. It will be understood by one of skill in the art that this is merely an exemplary configuration and any number of other configurations may be used in a similar manner.

In another embodiment, useful separately or together with the foregoing, the invention provides a set of junction oligonucleotides that may be used to facilitate connection of two or more pairs of construction polynucleotides using the junction assembly methods described above. The junction oligonucleotides comprise sequences that are complementary to at least a portion of two construction polynucleotides (or two portions of a construction polynucleotide for making tandem repeats). In one embodiment, the junction oligonucleotides may comprise a sequence that is complementary to the 5′ end of the medial sequence of a first construction polynucleotide and the 3′ end of the medial sequence of a second construction polynucleotide (see e.g., FIGS. 1-4). In another embodiment, the junction oligonucleotides may comprise a sequence that is complementary to the single stranded 3′ overhang of a first construction polynucleotide and the single stranded 5′ overhang of a second construction polynucleotide (see e.g., FIG. 5-7). In yet another embodiment, the junction oligonucleotide may comprise a sequence that is complementary to the single stranded 3′ overhang of one strand of a first construction polynucleotide, a sequence that is complementary to the single stranded 5′ overhang of one strand of a second construction polynucleotide, and a medial sequence that comprises a sequence complementary to at least 2 base pairs of the 3′ terminal portion of the medial sequence of the other strand of the first construction polynucleotide and at least 2 base pairs of the 5′ terminal portion of the medial sequence of the other strand of the second construction polynucleotide (see e.g., FIG. 7C). In certain embodiments, the junction oligonucleotides may additionally comprise 3′ and 5′ flanking sequences that contain primer binding sites, so as to permit their selective retrieval from a well containing plural junction oligonucleotides.

An exemplary embodiment of a set of junction oligonucleotides is illustrated in FIG. 13B. FIG. 13B shows two quadrants of a 384 well plate 22, 24 containing 96² junction oligonucleotides (or 9,216 oligonucleotides) and 96 sets of primers. For example, each well in quadrant 22 contains a mixture of 96 junction oligonucleotides and each well in quadrant 24 contains a single primer pair. As an example, the junction oligonucleotides may comprise sequences that are complementary to various combinations of the inner primers described above for FIG. 13A (e.g., complementary to the reverse inner primer from a first construction polynucleotide and the forward inner primer for a second construction polynucleotide). Each primer pair in quadrant 24 is designed to amplify a single junction oligonucleotide from each well in quadrant 22. Therefore, a single junction oligonucleotide may be amplified, and thus isolated, from a given mixture of junction oligonucleotides using one of the primer sets located in quadrant 24. The primer pairs may be complementary to the junction oligonucleotide sequences. Additionally, the junction oligonucleotides may contain 5′ and 3′ flanking regions. In an exemplary embodiment, the flanking regions of the junction oligonucleotides may contain binding sites for or more primer pairs. For example, the flanking regions may contain binding sites for the primer pairs in quadrant 24 that may be used to amplify, and thus isolate, a given junction oligonucleotide from the wells located in quadrant 22. Alternatively (or in addition), the flanking regions may contain binding sites for one or more sets of universal primers that may be used to amplify all of the junction oligonucleotides in a single well in quadrant 22, or all of the junction oligonucleotides in all of the wells in quadrant 22. In an exemplary embodiment, if the junction oligonucleotides contain 5′ and 3′ flanking regions, the regions may be removed using, for example, any of the methods described herein and as illustrated in FIGS. 11 and 12. In certain embodiments, one of the primers in a primer pair used to amplify the junction oligonucleotides may be functionalized with a group that facilitates isolation of one strand of the junction oligonucleotides, e.g., such as biotin (see e.g., FIG. 4). It will be understood by one of skill in the art that this is merely an exemplary configuration and any number of other configurations may be used in a similar manner.

Using a combination of the set of construction polynucleotides and junction oligonucleotides illustrated in FIGS. 13A and 13B it will possible to produce 96³×96² or 96⁵ different polynucleotide constructs each comprising the medial sequences from two construction polynucleotides. Therefore, a very large number of possible polynucleotide constructs may easily be constructed using, for example, only one and half 384 well plates. Furthermore, any two construction polynucleotide sequences may be connected together using the junction assembly methods described herein without needing to design around restriction enzyme sites, etc. Using the same set of construction polynucleotides and junction oligonucleotides, it is also possible to construct multiple and/or larger polynucleotide constructs optionally in a single pool. For example, it will possible to prepare a polynucleotide construct comprising the medial sequences from 2, 3, 4, 5, 6, 7, 8, 9, 10, 20, 25, 30, 50, or more construction polynucleotides. Additionally, it will be possible to prepare 2, 3, 4, 5, 6, 7, 8, 9, 10, 20, 25, 30, 50, or more polynucleotide constructs in a single reaction mixture.

As an alternative to the embodiment illustrated in FIG. 13, the construction and/or junction oligonucleotides may be isolated from a mixture of oligonucleotides using an affinity sequence. For example, in one embodiment, the 5′ and/or 3′ flanking sequences of the polynucleotides may comprise one or more aptamer sequences that may be used to isolate a given polynucleotide from a pool of polynucleotides using one or more rounds of isolation. Alternatively, the 3′ and/or 5′ flanking sequences may comprise sequences that permit isolation of a given polynucleotide based on selective hybridization with a complementary sequence using one or more rounds of isolation. For example, a hybridization sequence complementary to a 5′ and/or 3′ flanking sequence of at least a portion of the polynucleotides in a pool may be contacted with the pool under hybridization conditions. The hybridization sequence may be functionalized with biotin, or attached to a column or beads to permit separation of sequences that bind to the hybridization sequence from the remainder of the pool. The polynucleotides containing a 3′ and/or 5′ flanking sequence that binds to the hybridization sequence can then be isolated from the pool and subsequently separated from the hybridization sequence under denaturing conditions. This isolated pool may then be subjected to amplification and/or additional rounds of isolation with the same or a different hybridization sequence until a given poly or oligonucleotide has been isolated from the mixture. In certain embodiments, it may be desirable to use various combinations of the isolation methods described above, e.g., combinations of selective amplification and/or affinity isolation using an aptamer and/or selective hybridization. When using an affinity sequence to isolate an oligonucleotide from a mixture of oligonucleotides, the oligonucleotides may optionally be amplified before and/or after an isolation procedure.

In certain embodiments, the invention provides compositions comprising one or more construction polynucleotides, one or more junction oligonucleotides, and/or one or more primer pairs for amplifying the construction and/or junction oligonucleotides. In other embodiments, the invention provides multi-well plates comprising one or more construction polynucleotides, one or more junction oligonucleotides, and/or one or more primer pairs for amplifying the construction and/or junction oligonucleotides. The oligonucleotides and polynucleotides may be supplied in the same or separate wells. In yet another embodiment, the invention provides multi-well plates comprising a plurality of mixtures of construction polynucleotides; a plurality of mixtures of junction oligonucleotides and/or a plurality of primer pairs. In yet another embodiment, the invention provides substrates, such as chips, plates, beads, etc., having immobilized thereon, e.g., synthesized by known chemistries, one or more construction polynucleotides, one or more junction oligonucleotides, and/or one or more primer pairs. In an exemplary embodiment, such immobilized oligonucleotides are chemically synthesized on the substrate using the methods described further herein.

In certain embodiments, the invention provides kits for constructing one or more polynucleotide constructs. For example, the kits may comprise one or more construction polynucleotides, one or more junction oligonucleotides, and one or more primer pairs for amplifying the construction and/or junction oligonucleotides. The oligonucleotides and/or primers may be supplied in a single composition or in a plurality of compositions. In an exemplary embodiment, a plurality of construction polynucleotides, a plurality of junction oligonucleotides and/or a plurality of primer pairs may be supplied in a kit. Each of the sequences of the construction polynucleotides and/or junction oligonucleotides may be supplied as separate compositions or as one or more mixtures. In certain embodiments, the kits may additionally comprise instructions, a listing of the names and/or sequences of the oligonucleotide reagents in the kit, a multi-well plate, and/or one or more chemical or enzymatic reagents such as buffer, chemical ligation reagents, enzymatic ligation reagents, resolvase, uracil DNA glycosylase, an AP endonuclease, USER enzyme, an exonuclease, polymerase, dNTPs, biotin, avidin, beads, columns, etc.

In still another embodiment, the invention provides methods useful to bioengineers enabling them to design any conceivable DNA sequence, essentially and in principle of any desired length, to implement the design by synthesizing oligonucleotides and polynucleotides of a structure as disclosed herein, and then to join the polynucleotides together to produce the design. This can be executed manually using reagents described herein, e.g., by students with limited machinery in an academic or research laboratory, or preferably may be executed at higher throughput using automated machinery such as is described below.

5. Polynucleotide Synthesis

In various embodiments, the methods described herein utilize construction polynucleotides and/or junction oligonucleotides. The sequences of the construction polynucleotides may be essentially limitless as described further above. The sequences of the junction oligonucleotides may be determined based on the type of junction assembly method to be used and will be dependent upon the sequences of the construction polynucleotides. Preferably the flanking sequences of the construction and/or junction oligonucleotides and the sequences of the junction oligonucleotides themselves are designed to have as little nonspecific binding as possible. Design of the construction and/or junction oligonucleotides may be facilitated by the aid of a computer program such as, for example, DNAWorks (Hoover and Lubkowski, Nucleic Acids Res. 30: e43 (2002) or Gene2Oligo (Rouillard et al., Nucleic Acids Res. 32: W176-180 (2004) and world wide web at berry.engin.umich.edu/gene2oligo). In certain embodiments, it may be desirable to design a plurality of construction polynucleotide/junction oligonucleotide pairs to have substantially similar melting temperatures in order to facilitate manipulation of the plurality of polynucleotides in a single pool. This process may be facilitated by the computer programs described above. Normalizing melting temperatures between a variety of polynucleotide sequences may be accomplished by varying the length of the polynucleotides and/or by codon remapping the sequence (e.g., varying the A/T vs. G/C content in one or more polynucleotides without altering the sequence of a polynucleotide that may ultimately be encoded thereby) (see e.g., WO 99/58721).

In an exemplary embodiment, construction and/or junction oligonucleotides may comprise one or more sets of primer binding sites, including binding sites for universal primers that may be used for amplification of a pool of nucleic acids with one set, or a few sets, of primers. The sequence of the primer binding sites may be chosen to have an appropriate length and sequence to permit efficient primer hybridization and chain extension. Additionally, the sequence of the primer binding sites may be optimized so as to minimize non-specific binding to an undesired region of a nucleic acid in the pool. Design of primers and binding sites for the primers may be facilitated using a computer program such as, for example, DNA Works (supra) or Gene2Oligo (supra). In certain embodiments, it may be desirable to design several sets of primers/primer binding sites that will permit selective amplification of one or more nucleic acids in a given mixture.

Construction polynucleotides and/or junction oligonucleotides may be prepared by any method known in the art for preparation of polynucleotides having a desired sequence. For example, oligonucleotides may be isolated from natural sources, purchased from commercial sources, or designed from first principals. Preferably, oligonucleotides are synthesized using a method that permits high-throughput, parallel synthesis of multiple different sequences so as to reduce cost and production time and increase flexibility. In an exemplary embodiment, construction polynucleotides are themselves assembled from smaller construction oligonucleotides, and both the construction oligonucleotides and the junction oligonucleotides may be synthesized on a solid support in an array format, e.g., a microarray of single stranded DNA segments synthesized in situ on a common substrate wherein each oligonucleotide is synthesized on a separate feature or location on the substrate. Arrays may be constructed, custom ordered, or purchased from a commercial vendor. Various methods for constructing arrays are well known in the art. For example, methods and techniques applicable to synthesis of construction and/or junction oligonucleotide synthesis on a solid support, e.g., in an array format have been described, for example, in WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752 and Zhou et al., Nucleic Acids Res. 32: 5409-5417 (2004).

In an exemplary embodiment, construction and/or junction oligonucleotides may be synthesized on a solid support using maskless array synthesizer (MAS). Maskless array synthesizers are described, for example, in PCT application No. WO 99/42813 and in corresponding U.S. Pat. No. 6,375,903. Other examples are known of maskless instruments which can fabricate a custom DNA microarray in which each of the features in the array has a single stranded DNA molecule of desired sequence. The preferred type of instrument is the type shown in FIG. 5 of U.S. Pat. No. 6,375,903, based on the use of reflective optics. It is a desirable that this type of maskless array synthesizer is under software control. Since the entire process of microarray synthesis can be accomplished in only a few hours, and since suitable software permits the desired DNA sequences to be altered at will, this class of device makes it possible to fabricate microarrays including DNA segments of different sequence every day or even multiple times per day on one instrument. The differences in DNA sequence of the DNA segments in the microarray can also be slight or dramatic, it makes no different to the process. The MAS instrument may be used in the form it would normally be used to make microarrays for hybridization experiments, but it may also be adapted to have features specifically adapted for the compositions, methods, and systems described herein. For example, it may be desirable to substitute a coherent light source, i.e. a laser, for the light source shown in FIG. 5 of the above-mentioned U.S. Pat. No. 6,375,903. If a laser is used as the light source, a beam expanded and scatter plate may be used after the laser to transform the narrow light beam from the laser into a broader light source to illuminate the micro mirror arrays used in the maskless array synthesizer. It is also envisioned that changes may be made to the flow cell in which the microarray is synthesized. In particular, it is envisioned that the flow cell can be compartmentalized, with linear rows of array elements being in fluid communication with each other by a common fluid channel, but each channel being separated from adjacent channels associated with neighboring rows of array elements. During microarray synthesis, the channels all receive the same fluids at the same time. After the DNA segments are separated from the substrate, the channels serve to permit the DNA segments from the row of array elements to congregate with each other and begin to self-assemble by hybridization.

Other methods for synthesizing construction and/or junction oligonucleotides include, for example, light-directed methods utilizing masks, flow channel methods, spotting methods, pin-based methods, and methods utilizing multiple supports.

Light directed methods utilizing masks (e.g., VLSIPST methods) for the synthesis of oligonucleotides is described, for example, in U.S. Pat. Nos. 5,143,854, 5,510,270 and 5,527,681. These methods involve activating predefined regions of a solid support and then contacting the support with a preselected monomer solution. Selected regions can be activated by irradiation with a light source through a mask much in the manner of photolithography techniques used in integrated circuit fabrication. Other regions of the support remain inactive because illumination is blocked by the mask and they remain chemically protected. Thus, a light pattern defines which regions of the support react with a given monomer. By repeatedly activating different sets of predefined regions and contacting different monomer solutions with the support, a diverse array of polymers is produced on the support. Other steps, such as washing unreacted monomer solution from the support, can be used as necessary. Other applicable methods include mechanical techniques such as those described in U.S. Pat. No. 5,384,261.

Additional methods applicable to synthesis of construction and/or junction oligonucleotides on a single support are described, for example, in U.S. Pat. No. 5,384,261. For example reagents may be delivered to the support by either (1) flowing within a channel defined on predefined regions or (2) “spotting” on predefined regions. Other approaches, as well as combinations of spotting and flowing, may be employed as well. In each instance, certain activated regions of the support are mechanically separated from other regions when the monomer solutions are delivered to the various reaction sites.

Flow channel methods involve, for example, microfluidic systems to control synthesis of oligonucleotides on a solid support. For example, diverse polymer sequences may be synthesized at selected regions of a solid support by forming flow channels on a surface of the support through which appropriate reagents flow or in which appropriate reagents are placed. One of skill in the art will recognize that there are alternative methods of forming channels or otherwise protecting a portion of the surface of the support. For example, a protective coating such as a hydrophilic or hydrophobic coating (depending upon the nature of the solvent) is utilized over portions of the support to be protected, sometimes in combination with materials that facilitate wetting by the reactant solution in other regions. In this manner, the flowing solutions are further prevented from passing outside of their designated flow paths.

Spotting methods for preparation of oligonucleotides on a solid support involve delivering reactants in relatively small quantities by directly depositing them in selected regions. In some steps, the entire support surface can be sprayed or otherwise coated with a solution, if it is more efficient to do so. Precisely measured aliquots of monomer solutions may be deposited drop wise by a dispenser that moves from region to region. Typical dispensers include a micropipette to deliver the monomer solution to the support and a robotic system to control the position of the micropipette with respect to the support, or an ink-jet printer. In other embodiments, the dispenser includes a series of tubes, a manifold, an array of pipettes, or the like so that various reagents can be delivered to the reaction regions simultaneously.

Pin-based methods for synthesis of oligonucleotides on a solid support are described, for example, in U.S. Pat. No. 5,288,514. Pin-based methods utilize a support having a plurality of pins or other extensions. The pins are each inserted simultaneously into individual reagent containers in a tray. An array of 96 pins is commonly utilized with a 96-container tray, such as a 96-well microtitre dish. Each tray is filled with a particular reagent for coupling in a particular chemical reaction on an individual pin. Accordingly, the trays will often contain different reagents. Since the chemical reactions have been optimized such that each of the reactions can be performed under a relatively similar set of reaction conditions, it becomes possible to conduct multiple chemical coupling steps simultaneously.

In yet another embodiment, a plurality of construction and/or junction oligonucleotides may be synthesized on multiple supports. On example is a bead based synthesis method which is described, for example, in U.S. Pat. Nos. 5,770,358, 5,639,603, and 5,541,061. For the synthesis of molecules such as oligonucleotides on beads, a large plurality of beads are suspended in a suitable carrier (such as water) in a container. The beads are provided with optional spacer molecules having an active site to which is complexed, optionally, a protecting group. At each step of the synthesis, the beads are divided for coupling into a plurality of containers. After the nascent oligonucleotide chains are deprotected, a different monomer solution is added to each container, so that on all beads in a given container, the same nucleotide addition reaction occurs. The beads are then washed of excess reagents, pooled in a single container, mixed and re-distributed into another plurality of containers in preparation for the next round of synthesis. It should be noted that by virtue of the large number of beads utilized at the outset, there will similarly be a large number of beads randomly dispersed in the container, each having a unique oligonucleotide sequence synthesized on a surface thereof after numerous rounds of randomized addition of bases. An individual bead may be tagged with a sequence which is unique to the double-stranded oligonucleotide thereon, to allow for identification during use.

Various exemplary protecting groups useful for synthesis of oligonucleotides on a solid support are described in, for example, Atherton et al., 1989, Solid Phase Peptide Synthesis, IRL Press.

In various embodiments, the methods described herein utilize solid supports for immobilization of nucleic acids. For example, oligonucleotides may be synthesized on one or more solid supports. Additionally, selection oligonucleotides may be immobilized on a solid support to facilitate removal of synthesized oligonucleotides containing sequence errors and intended for assembly to form construction polynucleotides, as primers, or as junction oligonucleotides. Exemplary solid supports include, for example, slides, beads, chips, particles, strands, gels, sheets, tubing, spheres, containers, capillaries, pads, slices, films, or plates. In various embodiments, the solid supports may be biological, non-biological, organic, inorganic, or combinations thereof. When using supports that are substantially planar, the support may be physically separated into regions, for example, with trenches, grooves, wells, or chemical barriers (e.g., hydrophobic coatings, etc.). Supports that are transparent to light are useful when the assay involves optical detection (see e.g., U.S. Pat. No. 5,545,531). The surface of the solid support will typically contain reactive groups, such as carboxyl, amino, and hydroxyl or may be coated with functionalized silicon compounds (see e.g., U.S. Pat. No. 5,919,523).

In one embodiment, the oligonucleotides synthesized on the solid support may be used as a template for the production of construction polynucleotides and/or selection oligonucleotides for assembly into longer polynucleotide constructs. For example, the support bound oligonucleotides may be contacted with primers that hybridize to the oligonucleotides under conditions that permit chain extension of the primers. The support bound duplexes may then be denatured and subjected to further rounds of amplification.

In another embodiment, the support bound oligonucleotides may be removed from the solid support prior to assembly into polynucleotide constructs. The oligonucleotides may be removed from the solid support, for example, by exposure to conditions such as acid, base, oxidation, reduction, heat, light, metal ion catalysis, displacement or elimination chemistry, or by enzymatic cleavage.

In one embodiment, oligonucleotides may be attached to a solid support through a cleavable linkage moiety. For example, the solid support may be functionalized to provide cleavable linkers for covalent attachment to the oligonucleotides. The linker moiety may be of six or more atoms in length. Alternatively, the cleavable moiety may be within an oligonucleotide and may be introduced during in situ synthesis. A broad variety of cleavable moieties are available in the art of solid phase and microarray oligonucleotide synthesis (see e.g., Pon, R., Methods Mol. Biol. 20:465-496 (1993); Verma et al., Annu. Rev. Biochem. 67:99-134 (1998); U.S. Pat. Nos. 5,739,386, 5,700,642 and 5,830,655; and U.S. Patent Publication Nos. 2003/0186226 and 2004/0106728). A suitable cleavable moiety may be selected to be compatible with the nature of the protecting group of the nucleoside bases, the choice of solid support, and/or the mode of reagent delivery, among others. In an exemplary embodiment, the oligonucleotides cleaved from the solid support contain a free 3′-OH end. Alternatively, the free 3′-OH end may also be obtained by chemical or enzymatic treatment, following the cleavage of oligonucleotides. The cleavable moiety may be removed under conditions which do not degrade the oligonucleotides. Preferably the linker may be cleaved using two approaches, either (a) simultaneously under the same conditions as the deprotection step or (b) subsequently utilizing a different condition or reagent for linker cleavage after the completion of the deprotection step.

The covalent immobilization site may either be at the 5′ end of the oligonucleotide or at the 3′ end of the oligonucleotide. In some instances, the immobilization site may be within the oligonucleotide (i.e. at a site other than the 5′ or 3′ end of the oligonucleotide). The cleavable site may be located along the oligonucleotide backbone, for example, a modified 3′-5′ intemucleotide linkage in place of one of the phosphodiester groups, such as ribose, dialkoxysilane, phosphorothioate, and phosphoramidate internucleotide linkage. The cleavable oligonucleotide analogs may also include a substituent on, or replacement of, one of the bases or sugars, such as 7-deazaguanosine, 5-methylcytosine, inosine, uridine, and the like.

In one embodiment, cleavable sites contained within the modified oligonucleotide may include chemically cleavable groups, such as dialkoxysilane, 3′-(S)-phosphorothioate, 5′-(S)-phosphorothioate, 3′-(N)-phosphoramidate, 5′-(N)phosphoramidate, and ribose. Synthesis and cleavage conditions of chemically cleavable oligonucleotides are described in U.S. Pat. Nos. 5,700,642 and 5,830,655. For example, depending upon the choice of cleavable site to be introduced, either a functionalized nucleoside or a modified nucleoside dimer may be first prepared, and then selectively introduced into a growing oligonucleotide fragment during the course of oligonucleotide synthesis. Selective cleavage of the dialkoxysilane may be effected by treatment with fluoride ion. Phosphorothioate internucleotide linkage may be selectively cleaved under mild oxidative conditions. Selective cleavage of the phosphoramidate bond may be carried out under mild acid conditions, such as 80% acetic acid. Selective cleavage of ribose may be carried out by treatment with dilute ammonium hydroxide.

In another embodiment, a non-cleavable hydroxyl linker may be converted into a cleavable linker by coupling a special phosphoramidite to the hydroxyl group prior to the phosphoramidite or H-phosphonate oligonucleotide synthesis as described in U.S. Patent Application Publication No. 2003/0186226. The cleavage of the chemical phosphorylation agent at the completion of the oligonucleotide synthesis yields an oligonucleotide bearing a phosphate group at the 3′ end. The 3′-phosphate end may be converted to a 3′ hydroxyl end by a treatment with a chemical or an enzyme, such as alkaline phosphatase, which is routinely carried out by those skilled in the art.

In another embodiment, the cleavable linking moiety may be a TOPS (two oligonucleotides per synthesis) linker (see e.g., PCT publication WO 93/20092). For example, the TOPS phosphoramidite may be used to convert a non-cleavable hydroxyl group on the solid support to a cleavable linker. A preferred embodiment of TOPS reagents is the Universal TOPS™ phosphoramidite. Conditions for Universal TOPS™ phosphoramidite preparation, coupling and cleavage are detailed, for example, in Hardy et al, Nucleic Acids Research 22(15):2998-3004 (1994). The Universal TOPS™ phosphoramidite yields a cyclic 3′ phosphate that may be removed under basic conditions, such as the extended ammonia and/or ammonia/methylamine treatment, resulting in the natural 3′ hydroxy oligonucleotide.

In another embodiment, a cleavable linking moiety may be an amino linker. The resulting oligonucleotides bound to the linker via a phosphoramidite linkage may be cleaved with 80% acetic acid yielding a 3′-phosphorylated oligonucleotide.

In another embodiment, the cleavable linking moiety may be a photocleavable linker, such as an ortho-nitrobenzyl photocleavable linker. Synthesis and cleavage conditions of photolabile oligonucleotides on solid supports are described, for example, in Venkatesan et al. J. of Org. Chem. 61:525-529 (1996), Kahl et al., J. of Org. Chem. 64:507-510 (1999), Kahliet al., J. of Org. Chem. 63:4870-4871 (1998), Greenberg et al., J. of Org. Chem. 59:746-753 (1994), Holmes et al., J. of Org. Chem. 62:2370-2380 (1997), and U.S. Pat. No. 5,739,386. Ortho-nitrobenzyl-based linkers, such as hydroxymethyl, hydroxyethyl, and Fmoc-aminoethyl carboxylic acid linkers, may also be obtained commercially.

When synthesizing oligonucleotides on a solid support, the oligonucleotides at the edge of a particular location on the support tend to have a higher percentage of errors than the oligonucleotides located toward the center of that position. To increase the fidelity of the starting pool of construction and/or junction oligonucleotides it may be desirable to selectively release the oligonucleotides located toward the center of a location and minimize the oligonucleotides released from near the edges of a location. This may be accomplished using photolabile linking moieties for attachment of the oligonucleotides to the solid support. The oligonucleotides towards the center of the location may then be selectively removed by directing light to the center of the location. Highly accurate irradiation of the center of a location on a solid support may be achieved, for example, using a maskless array synthesizer or MAS (see e.g., PCT Publication WO99/42813 and U.S. Pat. No. 6,375,903). The MAS instrument may be used in the form it would normally be used to make microarrays for hybridization experiments, but it may also be adapted to have features specifically adapted for this application. For example, it may be desirable to use a coherent light source, i.e. a laser, to provide a narrow light beam and thus more accurate control over location of cleavage of the oligonucleotides.

In another embodiment, oligonucleotides may be removed from a solid support by an enzyme such as nucleases and/or glycosylases. A wide range of oligonucleotide bases, e.g. uracil, may be removed by a DNA glycosylase which cleaves the N-glycosylic bond between the base and deoxyribose, thus leaving an abasic site (Krokan et. al., Biochem. J. 325:1-16 (1997)). The abasic site in an oligonucleotide may then be cleaved by an AP endonuclease such as Endonuclease IV, leaving a free 3′-OH end. In another embodiment, oligonucleotides may be removed from a solid support upon exposure to one or more restriction endonucleases, including, for example, class IIs restriction enzymes. For example, a restriction endonuclease recognition sequence may be incorporated into the immobilized oligonucleotides and the oligonucleotides may be contacted with one or more restriction endonucleases to remove the oligonucleotides from the support. In various embodiments, when using enzymatic cleavage to remove the oligonucleotides from the support, it may be desirable to contact the single stranded immobilized oligonucleotides with primers, polymerase and dNTPs to form immobilized duplexes. The duplexes may then be contacted with the enzyme (e.g., restriction endonuclease, DNA glycosylase, etc.) to remove the duplexes from the surface of the support. Methods for synthesizing a second strand on a support bound oligonucleotide and methods for enzymatic removal of support bound duplexes are described, for example, in U.S. Pat. No. 6,326,489. Alternatively, short oligonucleotides that are complementary to the restriction endonuclease recognition and/or cleavage site (e.g., but are not complementary to the entire support bound oligonucleotide) may be added to the support bound oligonucleotides under hybridization conditions to facilitate cleavage by a restriction endonuclease (see e.g., PCT Publication No. WO 04/024886).

6. Amplification of Nucleic Acids

In various embodiments, the methods disclosed herein comprise amplification of nucleic acids including, for example, construction polynucleotides, junction oligonucleotides, and/or polynucleotide constructs. Amplification may be carried out during isolation of a construction and/or junction oligonucleotide from a pool of oligonucleotides and/or may be carried out after conducting a junction assembly method as a means to amplify and/or select the correct product. Amplification methods may comprise contacting a nucleic acid with one or more primers that specifically hybridize to the nucleic acid under conditions that facilitate hybridization and chain extension. Exemplary methods for amplifying nucleic acids include the polymerase chain reaction (PCR) (see, e.g., Mullis et al. (1986) Cold Spring Harb. Symp. Quant. Biol. 51 Pt 1:263 and Cleary et al. (2004) Nature Methods 1:241; and U.S. Pat. Nos. 4,683,195 and 4,683,202), anchor PCR, RACE PCR, ligation chain reaction (LC_{R}) (see, e.g., Landegran et al. (1988) Science 241:1077-1080; and Nakazawa et al. (1994) Proc. Natl. Acad. Sci. U.S.A. 91:360-364), self sustained sequence replication (Guatelli et al. (1990) Proc. Natl. Acad. Sci. U.S.A. 87:1874), transcriptional amplification system (Kwoh et al. (1989) Proc. Natl. Acad. Sci. U.S.A. 86:1173), Q-Beta Replicase (Lizardi et al. (1988) BioTechnology 6:1197), recursive PCR (Jaffe et al. (2000) J. Biol. Chem. 275:2619; and Williams et al. (2002) J. Biol. Chem. 277:7790), the amplification methods described in U.S. Pat. Nos. 6,391,544, 6,365,375, 6,294,323, 6,261,797, 6,124,090 and 5,612,199, or any other nucleic acid amplification method using techniques well known to those of skill in the art. In exemplary embodiments, the methods disclosed herein utilize PCR amplification.

As described above, the construction polynucleotides and/or selection oligonucleotides may be designed with primer binding sites for one or more sets of universal primers (see e.g., PCT Publication No. WO 04/024886). Alternatively, primer binding sites may be added to a nucleic acid after synthesis through the use of chimeric primers that contain a region complementary to the target nucleic acid and a non-complementary region that becomes incorporated during the amplification process (see e.g., WO 99/58721).

Primers suitable for use in the amplification methods disclosed herein may be designed with the aid of a computer program, such as, for example, DNA Works (supra) or Gene2Oligo (supra). Typically primers are from about 5 to about 500, about 10 to about 100, about 10 to about 50, or about 10 to about 30 nucleotides in length. In exemplary embodiments, a set of primers or a plurality of sets of primers may be designed so as to have substantially similar melting temperatures to facilitate manipulation of a complex reaction mixture. The melting temperature may be influenced, for example, by primer length and nucleotide composition.

In an exemplary embodiment, one or more primer binding sites may be designed to be removable using the methods described herein (see e.g., FIGS. 11 and 12).

In certain embodiments, it may be desirable to utilize a primer comprising one or more modifications such as a cap (e.g., to prevent exonuclease cleavage), a linking moiety (such as those described above to facilitate immobilization of an oligonucleotide onto a substrate), or a label (e.g., to facilitate detection, isolation and/or immobilization of a nucleic acid construct). Suitable modifications include, for example, various enzymes, prosthetic groups, luminescent markers, bioluminescent markers, fluorescent markers (e.g., fluorescein), radiolabels (e.g., ³²P, ³⁵S, etc.), biotin, polypeptide epitopes, etc. Based on the disclosure herein, one of skill in the art will be able to select an appropriate primer modification for a given application.

7. Sequencing/In Vivo Selection

In certain embodiments, it may be desirable to evaluate successful junction assembly of a synthetic polynucleotide construct by DNA sequencing, hybridization-based diagnostic methods, molecular biology techniques, such as restriction digest, selection marker assays, functional selection in vivo, or other suitable methods. For example, functional selection may be carried out by introducing a polynucleotide construct into a cell and assaying for expression of one or polynucleotides on the construct. Successful assemblies may be determined by assaying for a detectable marker, a selectable marker, a polypeptide of a given size (e.g., by size exclusion chromatography, gel electrophoresis, etc.), or by assaying for an enzymatic function of one or more polypeptides encoded by the polynucleotide construct. DNA manipulations and enzyme treatments are carried out in accordance with established protocols in the art and manufacturers' recommended procedures. Suitable techniques have been described in Sambrook et al. (2nd ed.), Cold Spring Harbor Laboratory, Cold Spring Harbor (1982, 1989); Methods in Enzymol. (Vols. 68, 100, 101, 118, and 152-155) (1979, 1983, 1986 and 1987); and DNA Cloning, D. M. Clover, Ed., IRL Press, Oxford (1985). In certain embodiments, the polynucleotide constructs may be introduced into an expression vector and transfected into a host cell. The host cell may be any prokaryotic or eukaryotic cell. For example, a polypeptide may be expressed in bacterial cells, such as E. coli, insect cells (baculovirus), yeast, plant, or mammalian cells. The host cell may be supplemented with tRNA molecules not typically found in the host so as to optimize expression of the polypeptide. Ligating the polynucleotide construct into an expression vector, and transforming or transfecting into hosts, either eukaryotic (yeast, avian, insect or mammalian) or prokaryotic (bacterial cells), are standard procedures. Examples of expression vectors suitable for expression in prokaryotic cells such as E. coli include, for example, plasmids of the types: pB_{R}322-derived plasmids, pEMBL-derived plasmids, pEX-derived plasmids, pBTac-derived plasmids and pUC-derived plasmids; expression vectors suitable for expression in yeast include, for example, YEP24, YIP5, YEP51, YEP52, pYES2, and YRP17; and expression vectors suitable for expression in mammalian cells include, for example, pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors.

### 8. EXEMPLARY EMBODIMENTS

The polynucleotide constructs that can be synthesized in accordance with the compositions and methods described herein are essentially unlimited in variety. The methods provided herein permit the researcher to develop nucleic acid (and corresponding polypeptide) sequences from first principles without being bound by the limitations of naturally occurring sequences, site directed mutagenesis, or random mutagenesis techniques.

In an exemplary embodiment, the invention provides compositions comprising construction polynucleotides having various types of sequences. For example, construction polynucleotides having the following types of sequences may be supplied in one or more mixtures: polynucleotides that encode peptides, proteins, protein fragments, protein domains, etc. or polynucleotides that contain regulatory sequences for DNA, RNA or polypeptide synthesis (e.g., initiation, elongation, termination, folding and error correction, modification and/or degradation). Examples of regulatory sequences include, for example, operator regions, ribosome binding sites, transcriptional terminator, origins of replication, integration sites, promoters, enhancers, shine delgarno sequences, an ATG start codon, stop codons, poly-A sites, restriction sites, plasmid sequences, transposon sequences, splicing sequences, centromere sequences, telomeres, etc. Sequences that encode for polypeptides or polypeptide fragments are essentially unlimited in variety and may include, for example, polypeptides involved in information storage and processing (e.g., translation, ribosomal structure, biogenesis, transcription, DNA replication, DNA recombination, DNA repair, etc.), cellular processes (e.g., cell division, chromosome partitioning, post-translational modification, protein turnover, chaperones, cell envelope biogenesis, outer membrane, cell motility and secretion, inorganic ion transport and metabolism, signal transduction, etc.), metabolism (e.g., energy production, energy conversion, carbohydrate transport and metabolism, amino acid transport and metabolism, nucleotide transport and metabolism, coenzyme metabolism, lipid metabolism, secondary metabolite biosynthesis, transport, and catabolism, etc.), detectable labels or tags (including, for example, antibiotic resistance sequences, subcellular localization tags, fluorescent proteins, affinity tags such as a His tag, FLAG tag, etc.),-functional or structural domains (e.g., zinc finger domains, kinase domains, antigen binding domains, etc.), and polypeptides having particular functions (e.g., integrases, kinases, DNA repair enzymes, etc.). Examples of sequences may be found, for example, on the world wide web at parts.mit.edu; sanger.ac.uk/Software/Pfam/; ncbi.nlm.nih.gov/COG/old/palox.cgi?fun=all; ncbi.nlm.nih.gov/, etc.

In another embodiment, the junction assembly methods described herein may be used to produce a polynucleotide construct comprising tandem repeats of the same or homologous sequences. For example, the junction assembly methods (FIGS. 1-7) disclosed herein may be used to produce a polynucleotide construct comprising two or more repeats of the same sequence (e.g., by joining two copies of the same construction polynucleotide), interspersed repeats of two or more sequences (e.g., repeating copies in a regular or irregular pattern of two or more construction polynucleotides), or tandem copies of homologous sequences. In another embodiment, the junction assembly methods disclosed herein may be used to assemble polynucleotide constructs having highly homologous sequences or regions of high (or identical) homology in a single pool. Creation of such highly homologous sequences using traditional methods may lead to unwanted products that arise due to cross hybridization between the related sequences. Such problems are minimized using the methods of the current invention because the joining reaction may be controlled by the flanking regions that can be designed to avoid any cross hybridization.

9. Implementation Systems and Methods

Provided herein are methods and systems to design one or more sets of construction polynucleotides, junction oligonucleotides, and/or primer sequences, and/or to design a junction assembly strategy, for producing one or a plurality of polynucleotide constructs using the methods described herein. Also provided are systems and apparatus for automated assembly of polynucleotide constructs from construction polynucleotides using the junction assembly methods disclosed herein.

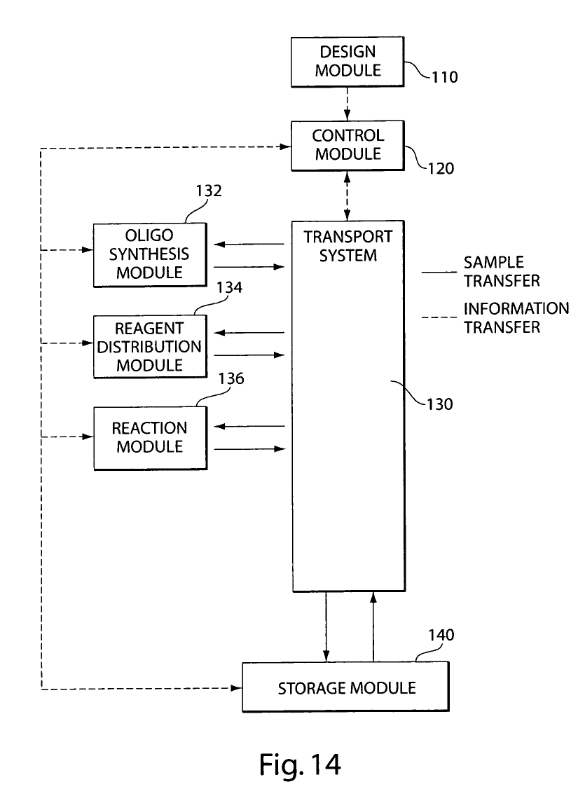

FIG. 14 shows an illustrative block diagram for one embodiment of a design module for carrying out the disclosed methods and systems. Using a user input device or another means, a user can input a sequence of a polynucleotide construct that is desired to be constructed and optionally other parameters. The user input device can be a processor-controlled device as provided herein, or can be provided with a user-interface that can allow a user or another to input information and/or data that can be used by the disclosed methods and systems. In various embodiments, the input sequence and/or parameters may be entered by the user or may be obtained from a database provided by the user, available over the internet, or available as part of the software program. Sequences and/or parameters obtained from a database may be provided by reference to a unique identifier rather than by input of the sequence and/or parameter itself. The user may optionally select a group of sequences to be joined together from a list that provides various sequences by name and/or function (e.g., promoter, terminator, etc.). Alternatively, the user may input a nucleic acid sequence (e.g., a DNA or RNA sequence) or may input a polypeptide sequence. When a polypeptide sequence is the input, the computer will reverse translate the sequence to produce one or more polynucleotide sequences that can encode the polypeptide sequence, e.g., exploiting codon preferences. For the purposes of discussion with respect to the illustrative embodiments, reference is made to a single input sequence, although it can be understood that the methods and systems can be applied to one or more input sequences where such sequences can be in a single and/or multiple databases, and thus such discussion is merely for convenience and can be understood to encompass or otherwise embody multiple input sequences.

The user entered information can be provided to one or more servers, where such servers can be understood to be associated with one or more processor controlled devices as provided herein. Such servers can include instructions for accepting the user-provided information and for accessing processor-executable instructions as provided herein for providing and/or otherwise designing construction polynucleotides, junction oligonucleotides, primer sequences, and/or an assembly strategy for preparing one or more polynucleotide constructs. The servers may access an oligonucleotide database that includes a list of construction polynucleotides, junction oligonucleotides and/or primers that have been produced and stored in an accessible manner. If all of the parts required to synthesize the desired polynucleotide construct are available, the system can construct an assembly protocol based on the accessible parts. Alternatively, if all of the parts are not available, the design module 110 can design construction polynucleotides, junction oligonucleotides, and/or primer pairs needed to produce the polynucleotide construct and optionally can direct their synthesis using an automated DNA synthesizer 12. The servers can have access to one or more databases which can include various types of information or analytical methods that may aid in polynucleotide design including, for example, methods for optimizing codon usage in a variety of host cells, methods for calculating melting temperature, methods for determining secondary structure of nucleic acid sequences, methods for identifying restriction endonuclease binding and/or cleavage sites, methods for identifying binding and/or enzymatic sites for other proteins, and/or methods for codon remapping sequences. The methods may be used to help design appropriate construction and/or junction oligonucleotides sequences to be synthesized or aid in the selection of appropriate construction and/or junction oligonucleotides from the database to be used in an assembly strategy. In one embodiment, the user can request use of one or more of such analysis methods when designing construction and/or junction oligonucleotides by providing the aforementioned user-specified information at a user device, where such information can be transmitted to a server(s) via a wired or wireless connection using one or more intranets and/or the internet, where the servers can thereafter process the request by accessing the databases. Such database accessing can include querying the databases based on the user information. Upon completing the requested query and/or analysis, the servers can provide the user-device with outputs and/or results that can be provided to a memory, the device display, or other location.

Those of ordinary skill in the art will recognize that the illustrative system can be understood to be representative of a client-server paradigm, where the instructions on the user device for obtaining user information and requesting a comparison can be a client, and the servers can be a server in the client-server paradigm.

Accordingly, it can be understood that the user device instructions and instructions on the servers can be included in a single device, where such embodiment may also be considered within the client-server paradigm. The user device can access, via wired or wireless communications and using one or more intranets and/or the internet, the databases for, querying, analyzing, and/or modifying sequences. Additionally, this embodiment can represent an embodiment that may not include a client-server paradigm.

With reference to FIG. 14, the design module 110 selects and/or designs sets of construction polynucleotides that will form the desired polynucleotide construct and junction oligonucleotides that may be used to connect the construction polynucleotides in a desired order. Optionally, the design module 110 may also select primer pairs that may be used to isolate and/or amplify the construction polynucleotides and/or junction oligonucleotides from a mixture of oligonucleotides in which they are stored. The design module contains a database that contains lists of the sequences of the construction polynucleotides, junction oligonucleotides and primer sequences and where they are located in a storage module 140. The design module produces an assembly protocol and outputs this to the control module 120 which may direct the automated assembly of the polynucleotide constructs. The assembly protocol may include steps to access the construction polynucleotides and/or junction oligonucleotides out of a storage system (including, e.g., PCR amplification/selection, affinity selection, etc.), steps for modifying the construction and/or junction oligonucleotides (e.g., making the oligonucleotides single stranded or producing single stranded overhangs), conducting a junction assembly method, selection/amplification of the correct product, etc. The control module 120 uses the assembly protocol from the design module and implements the strategy using an integrated storage module 140, reagent distribution module 134, and reaction module 136. The storage module 140 contains construction polynucleotides, junction oligonucleotides, and primer sequences that are stored in one or more addressable array configurations or logical accessible array configurations. The reagent distribution module 134 may contain a variety of reagents that may be useful for synthesis of oligonucleotides and/or assembly of polynucleotide constructs, including, for example, buffers, enzymes (e.g., polymerase, restriction endonucleases, UDG, AP endonuclease, USER, exonucleases, etc.), dNTPs, etc. The reaction module is used to carry out the assembly reaction and may include systems for controlling environmental conditions, including, for example, thermocycling for conducting PCR. The transport system 130 can transport materials between the different modules and contains an integrated fluid handling system for moving and mixing reagents as directed by the control module.

The integrated systems described herein may include, for example array elements, liquid handling elements, robotics (e.g., for moving microtiter plates) and the like. The system is based upon a set of modules as discussed above that are integrated for throughput and automation. The machine performs a number of tasks, using a liquid handling station, a PCR system, a plate/reservoir storage device and a robotic system for shuttling plates between the modules. This machine performs the entire shuffling process automatically, for example, in a microtiter plate format. For clarity of description, the system is split into a number of modules; however, module functions can be combined in practice to simplify the overall system. Typical integrated device elements include thermocyclic components, single and multi-well liquid handling, plate readers and plate handlers.

Sources, destinations and source and destination regions can be physically embodied in many different ways. For example, they can be microtiter wells or dishes, fritted microtiter trays (e.g., for coupling to column chromatographic methods) microfluidic systems, microchannels, containers, data structures, computer systems, combinations thereof, or the like. Examples of sources/destinations include solid phase arrays, liquid phase arrays, containers, microtiter trays, microtiter tray wells, microfluidic components, microfluidic chips, test tubes, centrifugal rotors, microscope slides, an organism, a cell, a tissue, and combinations thereof.

Movement means for moving nucleic acids and other reagents include fluid pressure modulators (e.g., pipettors or other pressure-driven channel systems), electrokinetic fluid force modulators, electroosmotic flow modulators, electrophoretic flow modulators, centrifugal force modulators, robotic armatures, pipettors, conveyor mechanisms, stepper motors, robotic plate manipulators, peristaltic pumps, magnetic field generators, electric field generators, fluid flow paths and the like. Fluid handling systems that may be used in connection with the systems disclosed herein are commercially available, including, for example, the Zymate systems from Zymark Corporation (Zymark Center, Hopkinton, Mass.) and other stations which utilize automatic pipettors, e.g., in conjunction with the robotics for plate movement (e.g., the ORCA.RTM. robot, which is used in a variety of laboratory systems available, e.g., from Beckman Coulter, Inc. (Fullerton, Calif.). Alternatively, fluid handling may be performed in microchips, e.g., involving transfer of materials from microwell plates or other wells through microchannels on the chips to destination sites (microchannel regions, wells, chambers or the like). Commercially available microfluidic systems include those from Hewlett-Packard/Agilent Technologies (e.g., the HP2100 bioanalyzer) and the Caliper High Throughput Screening System (see, e.g., world wide web at calipertech.com).

Any of a variety of array configurations can be used in the systems herein for storage of polynucleotides and oligonucleotides and/or other reagents. One common array format for use in the modules herein is a microtiter plate array, in which the array is embodied in the wells of a microtiter tray. Such trays are commercially available and can be ordered in a variety of well sizes and numbers of wells per tray, as well as with any of a variety of functionalized surfaces for binding of assay or array components. Common trays include the ubiquitous 96 well plate, with 384 and 1536 well plates also in common use. While arrays are most often thought of as physical elements with a specified spatial-physical relationship, the present invention can also make use of “logical” arrays, which do not have a straightforward spatial organization. For example, a computer system can be used to track the location of one or several components of interest which are located in or on physically disparate components. The computer system creates a logical array by providing a “look-up” table of the physical location of array members. Thus, even components in motion can be part of a logical array, as long as the members of the array can be specified and located.

The system may also be used to copy arrays of nucleic acids containing, for example, construction polynucleotides, junction oligonucleotides, and primers. The copy function may be used to produce duplicate arrays, master arrays, amplified arrays and the like, e.g., where any operation is contemplated which could make recovery of nucleic acids from an original array problematic (e.g. where a process to be performed consumes the original nucleic acids) or where a normalization of components (e.g., to provide similar concentrations of reactants or products) is useful. Copies can be made from master arrays, reaction mixture arrays or any duplicates thereof.

The devices and integrated systems optionally include any of a variety of component or module elements. These can include, e.g., one or more duplicates of the physical or logical array. A bar-code based sample tracking module, which includes a bar code reader and a computer readable database comprising at least one entry for at least one array or at least one array member can also be included, in which the entry is corresponded to at least one bar code. The device or integrated system can include a long term storage device such as a refrigerator; an electrically powered cooling device, a device capable of maintaining a temperature of <0° C., a freezer, a device which uses liquid nitrogen or liquid helium for cooling storing or freezing samples, a container comprising wet or dry ice, a constant temperature and/or constant humidity chamber or incubator; or an automated sample storage or retrieval unit. The device or integrated can also include one or more modules for moving arrays or array members into the long term storage device.

In various embodiments, software, or portions thereof, can be run in the RAM of general or special purpose computers or may be implemented in an application specific integrated circuit, digital signal processor, or other integrated circuit.

The methods and systems described herein are not limited to a particular hardware or software configuration, and may find applicability in many computing or processing environments. The methods and systems can be implemented in hardware or software, or a combination of hardware and software. The methods and systems can be implemented in one or more computer programs, where a computer program can be understood to include one or more processor executable instructions. The computer program(s) can execute on one or more programmable processors, and can be stored on one or more storage medium readable by the processor (including volatile and non-volatile memory and/or storage elements), one or more input devices, and/or one or more output devices. The processor thus can access one or more input devices to obtain input data, and can access one or more output devices to communicate output data. The input and/or output devices can include one or more of the following: Random Access Memory (RAM), Redundant Array of Independent Disks (RAID), floppy drive, CD, DVD, magnetic disk, internal hard drive, external hard drive, memory stick, or other storage device capable of being accessed by a processor as provided herein, where such aforementioned examples are not exhaustive, and are for illustration and not limitation.

The computer program(s) can be implemented using one or more high level procedural or object-oriented programming languages to communicate with a computer system; however, the program(s) can be implemented in assembly or machine language, if desired. The language can be compiled or interpreted.

As provided herein, the processor(s) can thus be embedded in one or more devices that can be operated independently or together in a networked environment, where the network can include, for example, a Local Area Network (LAN), wide area network (WAN), and/or can include an intranet and/or the internet and/or another network. The network(s) can be wired or wireless or a combination thereof and can use one or more communications protocols to facilitate communications between the different processors.

The processors can be configured for distributed processing and can utilize, in some embodiments, a client-server model as needed. Accordingly, the methods and systems can utilize multiple processors and/or processor devices, and the processor instructions can be divided amongst such single or multiple processor/devices.

The device(s) or computer systems that integrate with the processor(s) can include, for example, a personal computer(s), workstation (e.g., Sun, HP), personal digital assistant (PDA), handheld device such as cellular telephone, laptop, handheld, or another device capable of being integrated with a processor(s) that can operate as provided herein. Accordingly, the devices provided herein are not exhaustive and are provided for illustration and not limitation.

References to “a microprocessor” and “a processor”, or “the microprocessor” and “the processor,” can be understood to include one or more microprocessors that can communicate in a stand-alone and/or a distributed environment(s), and can thus can be configured to communicate via wired or wireless communications with other processors, where such one or more processor can be configured to operate on one or more processor-controlled devices that can be similar or different devices. Use of such “microprocessor” or “processor” terminology can thus also be understood to include a central processing unit, an arithmetic logic unit, an application-specific integrated circuit (IC), and/or a task engine, with such examples provided for illustration and not limitation.

Furthermore, references to memory, unless otherwise specified, can include one or more processor-readable and accessible memory elements and/or components that can be internal to the processor-controlled device, external to the processor-controlled device, and/or can be accessed via a wired or wireless network using a variety of communications protocols, and unless otherwise specified, can be arranged to include a combination of external and internal memory devices, where such memory can be contiguous and/or partitioned based on the application. Accordingly, references to a database can be understood to include one or more memory associations, where such references can include commercially available database products (e.g., SQL, Informix, Oracle) and also proprietary databases, and may also include other structures for associating memory such as links, queues, graphs, trees, with such structures provided for illustration and not limitation.

References to a network, unless provided otherwise, can include one or more intranets and/or the internet. References herein to microprocessor instructions or microprocessor-executable instructions, in accordance with the above, can be understood to include programmable hardware.

Unless otherwise stated, use of the word “substantially” can be construed to include a precise relationship, condition, arrangement, orientation, and/or other characteristic, and deviations thereof as understood by one of ordinary skill in the art, to the extent that such deviations do not materially affect the disclosed methods and systems.

Elements, components, modules, and/or parts thereof that are described and/or otherwise portrayed through the figures to communicate with, be associated with, and/or be based on, something else, can be understood to so communicate, be associated with, and or be based on in a direct and/or indirect manner, unless otherwise stipulated herein.

Certain illustrative embodiments of the systems and methods for carrying out the assembly methods described herein are described above. It will be understood by one of ordinary skill in the art that the systems and methods described herein can be adapted and modified to provide systems and methods for other suitable applications and that other additions and modifications can be made without departing from the scope of the systems and methods described herein.

Unless otherwise specified, the illustrated embodiments can be understood as providing exemplary features of varying detail of certain embodiments, and therefore, unless otherwise specified, features, components, modules, and/or aspects of the illustrations can be otherwise combined, separated, interchanged, and/or rearranged without departing from the disclosed systems or methods. Additionally, the shapes and sizes of components are also exemplary and unless otherwise specified, can be altered without affecting the scope of the disclosed and exemplary systems or methods of the present disclosure.

Although the methods and systems have been described relative to a specific embodiment thereof, they are not so limited. Obviously many modifications and variations may become apparent in light of the above teachings. Many additional changes in the details, materials, and arrangement of parts, herein described and illustrated, can be made by those skilled in the art. Accordingly, it will be understood that the following claims are not to be limited to the embodiments disclosed herein, can include practices otherwise than specifically described, and are to be interpreted as broadly as allowed under the law.

10. Automated System and Process for Custom-Designed Synthetic Polynucleotides

In one aspect, the present invention provides methods for interfacing computer technology with biological and chemical processing and synthesis equipment. In preferred embodiments, the present invention features methods for the computer to interface with equipment useful for biological and chemical processing and synthesis in a remote manner. Preferably, the methods of the present invention interface so as to run over a network or combination of networks such as the Internet, an internal network such as a company's own internal network, etc. thereby allowing the user to control the equipment remotely while maintaining a graphic display, updated in real time or near real time. Preferably, the methods of the present invention are used in conjunction with solid phase arrays that employ photolithographic or electrochemical methods for synthesis of chemical or biological materials.

In a second aspect, the present invention features a system for controlling and/or monitoring equipment for synthesizing or processing biological or chemical materials from a remote location. Such a system comprises a computer terminal remote from the equipment itself, software designed to monitor or control such equipment, and a communication means between the active part of such equipment and the computer terminal. Such a system preferably communicates between the computer terminal and the subject equipment via the internet or an internal intranet. Those skilled in the art readily understand that the software useful in such a system is highly specific depending upon the equipment itself and the parameter and conditions that need to be controlled or monitored to affect the desired processing or synthesis. As used herein, the term “remote” means not adjacent to. In effect, the term is used to denote that the computer terminal for effecting and monitoring the equipment may be located in the same vicinity as or in a completely location from the equipment. The present invention effectively-allows the artisan to process or synthesize biological or chemical materials using appropriate equipment in a location that is removed from the equipment itself. Moreover, the present invention allows the artisan to control or monitor more than one or a plurality of pieces of equipment from such a remote location.

The present invention may be applied in, but is not limited to, the fields of chemical or biological synthesis such as the preparation polynucleotide constructs. The methods of the present invention are especially applicable to such equipment as DNA synthesizers, thermocyclers, robotic instruments for controlled delivery of samples, etc. Such instruments may be controlled remotely according to the methods of the present invention thereby providing a graphic readout on progress and current status and controllable over a network.

The present invention provides a process for a manufacturer to obtain customer orders for custom-designed polynucleotides in an automated manner, comprising obtaining one or more desired sequence(s) from the customer, wherein the sequence(s) are polynucleotide sequences (e.g., DNA or RNA) or polypeptide sequences; selecting and/or designing a set of construction polynucleotides and/or junction oligonucleotides for production of the polynucleotides; designing a strategy for polynucleotide assembly using one of the junction assembly methods described herein. The assembly methods may include, for example, selecting and/or synthesizing the set of construction and/or junction oligonucleotides; one or more rounds of amplification to isolate the construction and/or junction oligonucleotides from a mixture or to produce a sufficient amount for assembly; and assembling the construction polynucleotides into the polynucleotide construct using a junction assembly method.

The step of designing a set of construction and/or junction oligonucleotides may comprise developing binding regions between complementary oligonucleotides (e.g., construction and junction oligonucleotides) according to consistent reaction conditions, wherein the reaction conditions include temperature, buffer conditions (including for example, pH and salt concentration), etc.

The construction and/or selection oligonucleotides may initially be synthesized on a solid support using any of a variety of methods for array synthesis such as, for example, in situ synthesis of oligonucleotides by spotting (e.g., inkjet methods), in situ synthesis of oligonucleotides by photolithography methods, electrochemical-based pH changes in situ synthesis of oligonucleotides, photochemical-based pH changes for in situ synthesis of oligonucleotides, maskless array synthesis methods, and combinations thereof. Copies of an array of construction and/or junction oligonucleotides may be produced by PCR.

The present invention further provides a system for a manufacturer to obtain customer orders for custom-designed polynucleotide constructs comprising a network-based receiving station for a manufacturer to receive desired polynucleotide and/or polypeptide sequences from the customer; a software means for selecting and/or designing a set of construction and/or junction oligonucleotides and/or designing an assembly strategy; and a manufacturing system for assembling the polynucleotide constructs. The software means may design the construction polynucleotides and/or junction oligonucleotides to provide substantially uniform melting temperatures, G/C vs. AT content, pH, environment, stringency conditions, or other conditions for consistent hybridization of oligonucleotide sequence(s). The software means may further design universal tags (including universal primer binding sites, nested primer binding sites, etc.) common to at least a portion of the construction and/or junction oligonucleotides. For example, the software may design primer binding sites and/or restriction endonuclease binding and cleavage sites to be added to flanking regions of the construction and/or selection oligonucleotides. The software may additional design primer sequences, select a restriction endonuclease, determine appropriate reaction conditions for PCR and/or enzyme digestion, etc. When assembling a plurality of constructs the software may additionally design an assembly strategy that permits assembly of a plurality of constructs in a single pool. Alternatively, the software may design a hierarchical assembly strategy for production of the polynucleotide constructs in parallel or serial reactions. In certain embodiments, the sequences for the set of construction and/or junction polynucleotides and/or the instructions for the assembly strategy may be retained within a storage device at the manufacturer. In certain embodiments, customers may be able to design a polynucleotide construct by selecting parts to be connected together from a database of parts. The parts may be selected based on sequence, name and/or function (e.g., a list or primers, a list of terminators, a list of fluorescent proteins, etc.).

Preferably, the design of construction and/or junction oligonucleotides comprises developing complementary binding regions between regions of various construction and/or junction oligonucleotides according to consistent reaction conditions, wherein the reaction conditions include temperature, pH, stringency, ionic strength, hydrophilic or hydrophobic environment, nucleotide content, oligonucleotide length, and combinations thereof wherein a software program having melting temperature, stringency and proton (pH) chemistry algorithms is employed. In an exemplary embodiment, the software program may also optimize sequences by codon remapping to remove and/or add one or more restriction endonuclease recognition and/or cleavage sites, to optimize or normalize expression in a particular expression system, and/or to reduce regions of secondary structure.

For example, a system may be employed whereby a researcher/customer designs a polynucleotide sequence using a computer at the remote (customer/researcher) location. The customer requests are transmitted to another computer that accesses at least one database to complete design of construction polynucleotides and/or junction oligonucleotides and/or an assembly strategy. Alternatively, the customer's remote computer may access at least one database during the design stage and send a complete design of construction polynucleotides and/or junction oligonucleotides and/or an assembly strategy to the local server. The local computer sends the complete design of construction polynucleotides and/or junction oligonucleotides and/or an assembly strategy to an automated fabrication unit. The polynucleotides are then assembled into the polynucleotide construct according to the assembly strategy. Preferably, the assembly takes places in a high-throughput and/or automated fashion using computer directed instruments such as thermalcyclers and/or robotic systems for sample mixing, etc.

The present invention further provides a user interface that a user can employ at a location that might be different from or remote from the site of manufacture of the array. This interface can provide the user with a way to specify the polynucleotide sequence to be synthesized, the degree of errors that will be tolerated for the desired application, the amount of polynucleotide that will be required, etc. The interface is deployed as a custom application that runs on a computer at the user's location, an applet that runs over a network, such as the Internet (such as with Java or Active X), a downloadable application, HTML forms, DHTML pages, XML forms, or any other technology that provides for interaction with the user and communication of data.

In a preferred embodiment, the synthesis of the polynucleotide construct is automated. A device (again, possibly at a site remote from the user) can take a specification for the polynucleotide sequence to be synthesized and produce the polynucleotide construct from that specification.

From a user's point of view, the user will first specify which polynucleotide sequences he or she is interested in synthesizing. Second, a server or servers (possibly with human intervention or help) will take the specification and design a set of construction and/or junction oligonucleotides, select a set a set of construction and/or junction oligonucleotides from a database, and/or design an assembly strategy. Third, the server will send instructions for assembly of the polynucleotide construct to an automated system as described above that contains reservoirs of construction polynucleotides, junction oligonucleotides, primers, and other reagents for assembly of polynucleotide constructs using a junction assembly method. The assembly strategy may involve multiple rounds of amplification and/or assembly. Fifth, after a polynucleotide construct is made that passes quality-control checks, the polynucleotide construct is shipped to the user.

The practice of the present methods will employ, unless otherwise indicated, conventional techniques of cell biology, cell culture, molecular biology, transgenic biology, microbiology, recombinant DNA, and immunology, engineering, robotics, optics, computer software and integration. The techniques and procedures are generally performed according to conventional methods in the art and various general references. which are within the skill of the art. Such techniques are explained fully in the literature. See, for example, Molecular Cloning A Laboratory Manual, 2^{nd} Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Patent No: 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. L. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunocherrical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986); Lakowicz, J. R. Principles of Fluorescence Spectroscopy, New York:Plenum Press (1983), and Lakowicz, J. R. Emerging Applications of Fluorescence Spectroscopy to Cellular Imaging: Lifetime Imaging, Metal-ligand Probes, Multi-photon Excitation and Light Quenching, Scanning Microsc. Suppl VOL. 10 (1996) pages 213-24, for fluorescent techniques, Optics Guide 5 Melles Griot.RTM. Irvine Calif. for general optical methods, Optical Waveguide Theory, Snyder & Love, published by Chapman & Hall, and Fiber Optics Devices and Systems by Peter Cheo, published by Prentice-Hall for fiber optic theory and materials.

Equivalents

The present invention provides among other things synthetic polynucleotide constructs and methods for producing synthetic polynucleotide constructs. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

Incorporation by Reference

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) (www.tigr.org) and/or the National Center for Biotechnology Information (NCBI) (www.ncbi.nlm.nih.gov).

What is claimed is:

1. A method of directed scarless ligation of a construction polynucleotide to another to produce a larger polynucleotide construct, the method comprising:
a) providing double stranded construction polynucleotides comprising a medial segment comprising a DNA sequence for inclusion within a said larger polynucleotide construct and left and right flanking terminal double stranded sequences of a length sufficient to permit selective hybridization of a complementary DNA thereto;
b) creating a single stranded overhang corresponding to a 3′ flanking region in a first said construction polynucleotide and to a 5′ flanking region in a second said construction polynucleotide while retaining the medial segments of said polynucleotides intact and free of residual flanking sequence bases;
c) contacting the construction polynucleotides under hybridization conditions with a junction oligonucleotide comprising sequence complementary to at least a portion of both said construction polynucleotides to form a complex wherein the two medial segments are aligned end to end; and
d) exposing the complex to ligation conditions, thereby forming a larger polynucleotide construct comprising fused said medial segments.

2. The method of claim 1, wherein step c) is conducted by contacting said first and second construction polynucleotides with a junction oligonucleotide complementary to at least a portion of the medial segments of both said construction polynucleotides.

3. The method of claim 1, wherein step c) is conducted by contacting said first and second construction polynucleotides with a junction oligonucleotide complementary to a 3′ flanking sequence of said first construction polynucleotide and a 5′ flanking sequence of said second construction polynucleotide.

4. The method of claim 3, wherein the construction polynucleotides and the junction oligonucleotide form a Holliday junction, the method further comprising contacting said Holiday junction with a resolvase.

5. The method of claim 1, wherein the left and right flanking terminal double stranded sequences of the construction polynucleotides comprise nested binding sites for two or more primer pairs.

6. The method of claim 5, further comprising providing a desired said construction polynucleotide by amplifying it selectively from a construction polynucleotide mixture using combination of two or more said primer pairs.

7. The method of claim 1, wherein at least five construction polynucleotides are joined together in a single reaction mixture.

8. The method of claim 1, wherein at least two larger polynucleotide construct are formed in a single reaction mixture.

9. The method of claim 1, further comprising amplifying the larger polynucleotide construct after step d) using primers complementary to the terminal flanking regions of said larger polynucleotide.

10. The method of claim 1, wherein a construction polynucleotide is coupled to a solid support.

11. The method of claim 10, wherein the construction polynucleotide is coupled to the solid support by a cleavable linker.

12. The method of claim 10, wherein the construction polynucleotide is coupled to the solid support by hybridization to an oligonucleotide attached to the support.

13. A method of producing a polynucleotide construct by joining together in a preselected order a selected pair of construction polynucleotides, the method comprising:
a) providing a mixture of different candidate construction polynucleotides comprising: a medial segment for joinder with another, flanked by 5′ and 3′ flanking sequences, wherein the flanking sequences comprise nested binding sites for two or more primer pairs;
b) providing a mixture of junction oligonucleotides comprising (i) a sequence that hybridizes to both a 5′ and a 3′ flanking sequence of at least one pair of construction polynucleotides, flanked 5′ and 3′ by (ii) junction oligonucleotide flanking sequences comprising binding sites for at least one pair of primers, thereby to enable amplification of a said junction oligonucleotide;
c) providing a plurality of primer pairs;
d) selecting at least a pair of construction polynucleotides from said mixture of candidate construction polynucleotides by amplification thereof with one or more of the primer pairs;
e) selecting a junction oligonucleotide from said mixture of junction oligonucleotides by amplification thereof with one or more of the primer pairs;
f) forming single stranded overhangs on the selected pair of construction polynucleotides thereby to produce a 3′ single stranded overhang corresponding to at least a portion of the 3′ flanking region of a first construction polynucleotide and a 5′ single stranded overhang corresponding to at least a portion of the 5′ flanking region of a second construction polynucleotide;
g) contacting the construction polynucleotide pair with their respective junction oligonucleotide under hybridization conditions to form a complex wherein the junction oligonucleotide is hybridized to the single stranded overhangs of the construction polynucleotides and the medial segments are aligned end to end; and
h) exposing the complex to ligation conditions, thereby to form a larger polynucleotide construct comprising fused said medial segments.

14. The method of claim 13, further comprising removing the 5′ and 3′ flanking regions from the junction oligonucleotide.

15. The method of claim 13, wherein the complex forms a Holliday junction.

16. The method of claim 15, further comprising contacting the complex with a resolvase.

17. A composition comprising a plurality of different junction oligonucleotides, each respective said junction oligonucleotides comprising a nucleotide sequence complementary to a 3′ terminal sequence of one construction polynucleotide and a nucleotide sequence complementary to a 5′ terminal sequence of another construction polynucleotide, said junction oligonucleotides having a sequence error rate less than about one base in 1000 so as to enable simultaneous selective hybridization of plural said junction oligonucleotides with their respective plural complementary construction polynucleotides and the preparation in parallel of multiple fusions between construction polynucleotides.

18. The composition of claim 17, wherein said junction oligonucleotides further comprise common removable primer binding sites on the ends thereof to permit amplification thereof with a pair of common primers.

19. The composition of claim 17, wherein said different junction oligonucleotides are immobilized on a surface, and are adapted for severance therefrom.

20. The composition of claim 17, wherein said junction oligonucleotides have a sequence error rate less than about one base in 1500, 2000, 3000, 5000, or 10,000 bases.
